SEQUENCE LISTING (83 sequences)

SEQ ID NO: 1 - DNA, 9365 bp, Zea mays
  misc_feature (1)..(9365): sh2 genomic DNA
  misc_feature (1)..(86): exon 1
  misc_feature (638)..(728): exon 2
  misc_feature (2231)..(2280): exon 3
  misc_feature (2710)..(2799): exon 4
  misc_feature (2943)..(3069): exon 5
  misc_feature (3509)..(3733): exon 6
  misc_feature (4208)..(4330): exon 7
  misc_feature (4615)..(4782): exon 8
  misc_feature (5019)..(5109): exon 9
  misc_feature (5201)..(5286): exon 10
  misc_feature (5371)..(5428): exon 11
  misc_feature (5530)..(5623): exon 12
  misc_feature (5790)..(5905): exon 13
  misc_feature (5974)..(6042): exon 14
  misc_feature (6212)..(6292): exon 15
  misc_feature (6458)..(6544): exon 16
  misc_feature (6615)..(6719): exon 17
  misc_feature (8607)..(8713): exon 18
  misc_feature (8796)..(8856): exon 19
  misc_feature (8973)..(9365): exon 20
atgggcgggcagtgctgtgagttagagcagtgtggcaatgttggtggtagtggtgacacccttcgggagcggaaggaggctggaacttcaggtcagttctattgccataaataaataaaataaaataaatgaggctcaaaattccaaaatgtcatttcagcgtgtgcaccgcttcataaggccagtctcgtgaatcctacaagtttgatggtattctgggcctcgggcatagcccatttatcataaataaggacaattttgtaattctctccttttctttttgtttgcttgtatttgtatttcacatgggactcacttatgttgatgtattttagcttgtaaaaaagagtaaaaacattattcagtgaagtgacgcattgagttcatgattaagtttcaattatcctttttaataggtccatgccacgcttgtataaggattgagtaaaaaaactaactttattacaccacagtagataacaataagtcatatcagttgcatcacacaagcacactatttacttcttgtttttggatcaattctttggtgtctgtaaacccatacaacataacttgaatatgaacagcactgcagggataattgaaacctaggagtaaataaaatggatgtgaacgtttcagattatttggcattctgtaaaagacccaacacccaaatgcttgaaaaaatatatcatgttgtaaaaattcgatctgcatttccttctgtaagcaataaaacatggctcttttaagaattaccttgacaaaaaggtaaaatataatatcaataataactccggataggaaaaacttattcttctgcttataattatatcttaaatttaagtctatgacaatgtgaaatatttattgctagtgctacattttaatgtgtaatgaaagcactttcttttggacattcgaattctcctttgaaaatgatatactcctgtgattctggtttttaggagaattgcatgcatggcctagtggtagcgttgagataatgagttaagctttttgaagtttctaatgtgaattgtttttcttttaacttaaatactcattcagcgaaagatcctctagcgtaatgggtaaggattccgagtatcaccttcaggttctgggttcgaccctcatcgggggcgaatttcgggcttggttaaaaaaaatactctcactatgccccgcccgctcccgggttacgttctgtgcgccaccctctggctgagctgttgtaaagtgggcgatgacggcccgctagtgatggggggcaggggtggacccaagtacggctgagtgagggcacatgcccccgcccatttctttgttctaagtactagggtctaaattttcaccgtgagcccccctgcttagtctaatttagatgcccttgctctggtattttgatgctcgtccttacaagtatgcccttagtcatattttgtcctgggtccgcccttgatgagaggggccagggtttagagattttctcggccgggaccaatgttccggtctcttcttaatatagtaccgggacagtctttccctctccggccgagttttttatactcactctaggtgcaaacgtaagttgttcaggcttgtgcttaaggattaagaaagtaaataaaatgaccatgttacactttttttatttataccgtatcgggaaagataaaccatttatctgtgagtggtagcatggtcaaggagaaaaaaatagatgcatagaggttctgggacgactcacatttagagcatagttgaaaaggctaaaacaaccaggagggggtggggtcgggatacataaattttcatgagtgatggttgaactattgagggtgcaaaataattttttcaacaaataatgtggtgaaatacctaagaggggtgcacctagcatagattttttggggctccccttggcctctcctttcttccgccctgaaaacaacctacatggatacatctgcaaccagagggagtatctgatgctttttcctgggcagggagagctatgaggcgtatgtcctcaaagccactttgcattgtgtgaaaccaatatcgatctttgttacttcatcatgcgtgaacatttgtggaaactactagcttacaagcattagtgacagctcagaaaaaagttatctctgaaaggtttcatgtgtaccgtgggaaatgagaaatgttgccaactcaaacaccttcaatatgttgtttgcaggcaaactcttctggaagaaaggtgtctaaaactatgaacgggttacagaaaggtataaaccacggctgtgcattttggaagtatcatctatagatgtctgttgaggggaaagccgtacgccaacgttatttactcagaaacagcttcaacacacagttgtctgctttatgatggcatctccacccaggcacccaccatcacctatctctcgtgcctgtttattttcttgccctttctgatcataaaaaatcattaagagtttgcaaacatgcataggcatatcaatatgctcatttattaatttgctagcagatcatcttcctactctttactttatttattgtttgaaaaatatgtcctgcacctagggagctcgtatacagtaccaatgcatcttcattaaatgtgaatttcagaaaggaagtaggaacctatgagagtatttttcaaaattaattagcggcttctattatgtttatagcaaaggccaagggcaaaattggaacactaatgatggttggttgcatgagtctgtcgattacttgcaagaaatgtgaacctttgtttctgtgcgtgggcataaaacaaacagcttctagcctcttttacggtacttgcacttgcaagaaatgtgaactccttttcatttctgtatgtggacataatgccaaagcatccaggctttttcatggttgttgatgtctttacacagttcatctccaccagtatgccctctcatactctatataaacacatcaacagcatcgcaattagccacaagatcacttcgggaggcaagtgcgatttcgatctcgcagccaccttttttttgttctgttgtaagtatactttcccttaccatctttatctgttagtttaatttgtaattgggaagtattagtggaaagaggatgagatgctatcatctatgtactctgcaaatgcatctgacgttatatgggctgcttcatataatttgaattgctccattcttgccgacaatatattgcaaggtatatgcctagttccatcaaaagttctgttttttcattctaaaagcattttagtggcacgcattttttgtccatgagggaaaggaaatctgttttggttactttgcttgaggtgcattcttcatatgtccagttttatggaagtaataaacttcagtttggtcataagatgtcatattaaagggcaaacatatattcaatgttcaattcatcgtaaatgttccctttttgtaaaagattgcatactcatttatttgagttgcaggtgtatctagtagttggaggagatatgcagtttgcacttgcattggacacgaactcaggtcctcaccagataagatcttgtgagggtgatgggattgacaggttggaaaaattaagtattgggggcagaaagcaggagaaagctttgagaaataggtgctttggtggtagagttgctgcaactacacaatgtattcttacctcagatgcttgtcctgaaactcttgtaagtatccacctcaattattactcttacatgttggtttactttacgtttgtcttttcaagggaaatttactgtattttttgggttttgtgggagttctatacttctgttggactggttattgtaaagatttgttcaaatagggtcatctaataattgtttgaaatctgggaactgtggtttcactgcgttcaggaaaaagtgaattcttggttactgcatgaataacttatggaaatagaccttagagttgctgcatgattatcacaaatcattgctacgatatcttataatagttctttcgacctcgcattacatatataactgcaactcgtagttgcgttcaaaaaaaaatgcaactcttagaacgctcaccagtgtaatctttcctgaattgttatttaatggcatgtatgcactacttgtatacttatctaggattaagtaatctaactctaggccccatatttgcagcattctcaaacacagtcctctaggaaaaattatgctgatgcaaaccgtgtatctgctatcattttgggcggaggcactggatctcagctctttcctctgacaagcacaagagctacgcctgctgtaagggataacactgaacatccaacgttgattactctattatagtattatacagactgtacttttcgaatttatcttagttttctacaatatttagtggattcttctcattttcaagatacacaattgatccataatcgaagtggtatgtaagacagtgagttaaaagattatattttttgggagacttccagtcaaattttcttagaagtttttttggtccagatgttcataaagtcgccgctttcatactttttttaattttttaattggtgcactattaggtacctgttggaggatgttacaggcttattgatatccctatgagtaactgcttcaacagtggtataaataagatatttgtgatgagtcagttcaattctacttcgcttaaccgccatattcatcgtacataccttgaaggcgggatcaactttgctgatggatctgtacaggtgatttacctcatcttgttgatgtgtaatactgtaattaggagtagatttgtgtggagagaataataaacagatgccgagattcttctctaaaagtctagatccaaaggcattgtggttcaaaacactatggacttctaccatttatgtcattactttgccttaatgttccattgaatggggcaaattattgattctacaagtgtttaattaaaaactaattgttcatcctgcaggtattagcggctacacaaatgcctgaagagccagctggatggttccagggtacagcagactctatcagaaaatttatctgggtactcgaggtagttgatattttctcgtttatgaatgtccattcactcattcctgtagcattgtttctttgtaattttgagttctcctgtatttctttaggattattacagtcacaaatccattgacaacattgtaatcttgagtggcgatcagctttatcggatgaattacatggaacttgtgcaggtatggtgttctcttgttcctcatgtttcacgtaatgtcctgattttggattaaccaactacttttggcatgcattatttccagaaacatgtcgaggacgatgctgatatcactatatcatgtgctcctgttgatgagaggtaatcagttgtttatatcatcctaatatgaatatgtcatcttgttatccaacacaggatgcatatggtctaatctgctttcctttttttcccttcggaagccgagcttctaaaaatgggctagtgaagattgatcatactggacgtgtacttcaattctttgaaaaaccaaagggtgctgatttgaattctatggttagaaattccttgtgtaatccaattcttttgttttcctttctttcttgagatgaacccctcttttagttatttccatggataacctgtacttgacttattcagaaatgattttctattttgctgtagaatctgacactaaagctaatagctactgatgttgcagagagttgagaccaacttcctgagctatgctatagatgatgcacagaaatatccataccttgcatcaatgggcatttatgtcttcaagaaagatgcacttttagaccttctcaagtaatcactttcctgtgacttttttctatccaactcctagtttaccttctaacagtgtcaattcttaggtcaaaatatattcaattacatgactttggatctgaaatcctcccaagagctgtactagatcatagtgtgcaggtaagtctgatctgtctggagtatgtgttctgtaaactgtaaattcttcatgtcaaaaagttgtttttgtttccagtttccactagtttttatttaccaatgcacgatttatgtattttcgcttccatgcatcatacatactaacaatacattttacgtattgtgttaggcatgcatttttacgggctattgggaggatgttggaacaatcaaatcattctttgatgcaaacttggccctcactgagcaggtactctgtcatgtattctgtactgcatatatattacctggaattcaatgcatagaatgtgttagaccatcttagttccatcctgttttcttcaattagcttatcatttaatagttgttgactagaatctaaacacaaatttacctaatatgtttctctcttcagccttccaagtttgatttttatgatccaaaaacacctttcttcactgcaccccgatgcttgcctccgacgcaattggacaagtgcaaggtatatgtcttaccgagcacaattgttacctgagcaagattttgtgtacttgacttgttctcctccacagatgaaagatgcatttatctcagatggttgcttactgagagaatgcaacatcgagcattctgtgattggagtctgctcacgtgtcagctctggatgtgaactcaaggtacatactctgccaatgtatatgctgatgttttatacattctcttgcataatttgattcgagtcaccacaattagtgtaactgcaatctactcttgagtataccatttcaacaccaggcatcaccaaatcacacagaacaatagcaacgaagccttttagttccaagaaatttagggtagcctagagttgaaatctaacaaaacaaaagtcaaagctctatcacgtggatagttgttttccatgcactcttatttaagctaaatttttgggtatactacatccatttaattattgttttattgcttcttccctttgcctttcccccattactatcgcgtcttaagatcatactacgcactagtgtctttagaggtctctggtggacatgttcaaaccatctcaatcgaatcggtgttggacaagtttttcttgaatttgtgctacacctaacctatcacgtatatcatcgtttcaaacttgatccttcttgtatcatcataaatccaatgcaacatacgcatttctgcaacatttatctgttgaacatgtcatctttttgtaggttaacattatgcaccatacaatgtagcatgtctaatcatcatcctataaaatttacattttagcttatgtggtatcctcttgccacttagaacatcatatgtttgatgccatttcatccaccctgctttgattctatggctaacatcttcattaatatcctcgcctctctgtatcattggtcctaaatatggaaatacattctttctgggcactacttgaccttccaaactaacgtctcctttgatcctttcttgtgtgtagtagtaccgaagtcacatctcatatattcggttttagttctactaagtcccgggttcgatccccctcaggggtaaatttcgggcttggtaaaaaaaatcccctcgctgtgtcccgcccgctctcggggatcgatatcctgcgcgccaccctccggctgggcattgcagagtgggcagttgatcggctcgttagtgatggggagcggggttcaagggttttctcggccgggaccatgtttcggtctcttaatataataccgggagggcagtctttccctccccggtcgagttttagttctaccgagtctaaaacctttggactctagagtcccctgtcacaactcacaactctagttttctatttacttctacctagcgtttattaatgatcactatatcgtctgtaaaaagcatacaccaaggtaatccccttgtatgtcccttgtaatattatccatcacaagaaaaaaaaggtaaggctcaaagttgacttttgatataatcctattctaatcgagaagtcatctgtatcttcgtctcttgttcgaacactagtcacaattttttttgtacatgttcttaatgagtccaacgtaatattccttgatattttgtaagccctcatcaagtcaatgaaaatcacgtgtaggtccttcatttgttccttatactgctccatcacttgtctcattaagaaaatatctctcatagttaaccttttggcatgaaacaaaatcacacagaagttgtttcctttttttaagatcccacacaaaagaggtttgatctaaggaatctggatccctgacaggtttatcaaaatcctttgtgtttttcttaaaactgaatattcctccagcttctagtattgatgtaatattcaatctgtttagcaagtgaacaccttggttcttgttgttactgtacatcccacccacccccccgaggcccagattaccacgacatgaatacaagaatattgaacccagatctagagtttgtttgtactgttgaaaatcggtgacaattcattttgttattgcgctttctgataacgacaggactccgtgatgatgggagcggacacctatgaaactgaagaagaagcttcaaagctactgttagctgggaaggtcccagttggaataggaaggaacacaaagataaggtgagtatggatgtggaaccaccggttagttcccaaaaatatcactcactgatacctgatggtatcctctgattattttcaggaactgtatcattgacatgaatgctaggattgggaagaacgtggtgatcacaaacagtaaggtgagcgagcgcacctacatgggtgcagaatcttgtgtgctcatctatcctaattcggtaattcctatccagcgctagtcttgtgaccatggggcatgggttcgactctgtgacagggcatccaagaggctgatcacccggaagaagggtactacataaggtctggaatcgtggtgatcttgaagaatgcaaccatcaacgatgggtctgtcatatagatcggctgcgtttgcgtctacaaaacaagaacctacaatggtattgcatcgatggatcgtgtaaccttggtatggtaagagccgcttgacaggaagtcgagcgttcgggcgcaagatgcgttgtctggcatgctgttccttgaccatttgtgctgctagtatgtactgttataagctgccctagaagttgcagcaaacctttttatgaacctttgtatttccattacctgctttggatcaactatatctgtcatcctatatattactaaatttttacgtgtttttctaa

SEQ ID NO: 2 - DNA, 2308 bp, Zea mays
  misc_feature (1)..(2308): sh2 cDNA
atgggcgggcagtgctgtgagttagagcagtgtggcaatgttggtggtagtggtgacacccttcgggagcggaaggaggctggaacttcagattatttggcattctgtaaaagacccaacacccaaatgcttgaaaaaatatatcatgttgtaaaaattcgatctgcatttccttctgcaaactcttctggaagaaaggtgtctaaaactatgaacgggttacagaaagcaaaggccaagggcaaaattggaacactaatgatggttggttgcatgagtctgtcgattacttgcaagaaatgtgaacctttgtttctttcatctccaccagtatgccctctcatactctatataaacacatcaacagcatcgcaattagccacaagatcacttcgggaggcaagtgcgatttcgatctcgcagccaccttttttttgttctgttgtgtatctagtagttggaggagatatgcagtttgcacttgcattggacacgaactcaggtcctcaccagataagatcttgtgagggtgatgggattgacaggttggaaaaattaagtattgggggcagaaagcaggagaaagctttgagaaataggtgctttggtggtagagttgctgcaactacacaatgtattcttacctcagatgcttgtcctgaaactcttcattctcaaacacagtcctctaggaaaaattatgctgatgcaaaccgtgtatctgctatcattttgggcggaggcactggatctcagctctttcctctgacaagcacaagagctacgcctgctgtacctgttggaggatgttacaggcttattgatatccctatgagtaactgcttcaacagtggtataaataagatatttgtgatgagtcagttcaattctacttcgcttaaccgccatattcatcgtacataccttgaaggcgggatcaactttgctgatggatctgtacaggtattagcggctacacaaatgcctgaagagccagctggatggttccagggtacagcagactctatcagaaaatttatctgggtactcgaggattattacagtcacaaatccattgacaacattgtaatcttgagtggcgatcagctttatcggatgaattacatggaacttgtgcagaaacatgtcgaggacgatgctgatatcactatatcatgtgctcctgttgatgagagccgagcttctaaaaatgggctagtgaagattgatcatactggacgtgtacttcaattctttgaaaaaccaaagggtgctgatttgaattctatgagagttgagaccaacttcctgagctatgctatagatgatgcacagaaatatccataccttgcatcaatgggcatttatgtcttcaagaaagatgcacttttagaccttctcaagtcaaaatatattcaattacatgactttggatctgaaatcctcccaagagctgtactagatcatagtgtgcaggcatgcatttttacgggctattgggaggatgttggaacaatcaaatcattctttgatgcaaacttggccctcactgagcagccttccaagtttgatttttatgatccaaaaacacctttcttcactgcaccccgatgcttgcctccgacgcaattggacaagtgcaagatgaaagatgcatttatctcagatggttgcttactgagagaatgcaacatcgagcattctgtgattggagtctgctcacgtgtcagctctggatgtgaactcaaggactccgtgatgatgggagcggacacctatgaaactgaagaagaagcttcaaagctactgttagctgggaaggtcccagttggaataggaaggaacacaaagataaggaactgtatcattgacatgaatgctaggattgggaagaacgtggtgatcacaaacagtaagggcatccaagaggctgatcacccggaagaagggtactacataaggtctggaatcgtggtgatcttgaagaatgcaaccatcaacgatgggtctgtcatatagatcggctgcgtttgcgtctacaaaacaagaacctacaatggtattgcatcgatggatcgtgtaaccttggtatggtaagagccgcttgacaggaagtcgagcgttcgggcgcaagatgcgttgtctggcatgctgttccttgaccatttgtgctgctagtatgtactgttataagctgccctagaagttgcagcaaacctttttatgaacctttgtatttccattacctgctttggatcaactatatctgtcatcctatatattactaaatttttacgtgtttttctaa

SEQ ID NO: 3 - protein, 672 aa, Zea mays
  MISC_FEATURE (1)..(672): sh2 native protein
MGGQCCELEQCGNVGGSGDTLRERKEAGTSDYLAFCKRPNTQMLEKIYHVVKIRSAFPSANSSGRKVSKTMNGLQKAKAKGKIGTLMMVGCMSLSITCKKCEPLFLSSPPVCPLILYINTSTASQLATRSLREASAISISQPPFFCSVVYLVVGGDMQFALALDTNSGPHQIRSCEGDGIDRLEKLSIGGRKQEKALRNRCFGGRVAATTQCILTSDACPETLHSQTQSSRKNYADANRVSAIILGGGTGSQLFPLTSTRATPAVPVGGCYRLIDIPMSNCFNSGINKIFVMSQFNSTSLNRHIHRTYLEGGINFADGSVQVLAATQMPEEPAGWFQGTADSIRKFIWVLEDYYSHKSIDNIVILSGDQLYRMNYMELVQKHVEDDADITISCAPVDESRASKNGLVKIDHTGRVLQFFEKPKGADLNSMRVETNFLSYAIDDAQKYPYLASMGIYVFKKDALLDLLKSKYIQLHDFGSEILPRAVLDHSVQACIFTGYWEDVGTIKSFFDANLALTEQPSKFDFYDPKTPFFTAPRCLPPTQLDKCKMKDAFISDGCLLRECNIEHSVIGVCSRVSSGCELKDSVMMGADTYETEEEASKLLLAGKVPVGIGRNTKIRNCIIDMNARIGKNVVITNSKGIQEADHPEEGYYIRSGIVVILKNATINDGSVI

SEQ ID NO: 4 - protein, 527 aa, Solanum tuberosum
  MISC_FEATURE (1)..(527): S. tuberosum AGPase large subunit
  MISC_FEATURE (1)..(66): chloroplast transit peptide
  MISC_FEATURE (67)..(527): mature AGPase large subunit
MDTCCVPMKSTVHLGRVSTGGFNNGEKEFFGEKIRGSLNSNLRINQLSKSLKLEKKENKIKPGVAYSVITTENDTQTVFVDMPRLERRRANPKDVAAVILGGGEGTKLFPLTSRTATPAVPVGGCYRLIDIPMSNCINSAINKIFVLTQYNSAPLNRHIARTYFGNGVSFGDGFVEVLAATQTPGEAGKKWFQGTADAVRKFIWVFEDAKNKNIENIVVLSGDHLYRMDYMELVQNHIDRNADITLSCAPAEDSRASDFGLVKIDSRGRVVQFAEKPKGFDLKAMQVDTTLVGLSPQDAKKSPYIASMGVYVFKTDVLLKLLKWSYPTSNDFGSEIIPAAIDDYNVQAYIFKDYWEDIGTIKSFYNASLALTQEFPEFQFYDPKTPFYTSPRFLPPTKIDNCKIKDAIISHGCFLRDCSVEHSIVGERSRLDCGVELKDTFMMGADYYQTESEIASLLAEGKVPIGIGENTKIRKCIIDKNAKIGKNVSIINKDGVQEADRPEEGFYIRSGIIIILEKATIRDGTVI

SEQ ID NO: 5 - DNA, 9365 bp, Zea mays
  misc_feature (1)..(9365): modified sh2 genomic DNA
  misc_feature (4283)..(4285): T249K modification
  misc_feature (4292)..(4294): Q252G modification
atgggcgggcagtgctgtgagttagagcagtgtggcaatgttggtggtagtggtgacacccttcgggagcggaaggaggctggaacttcaggtcagttctattgccataaataaataaaataaaataaatgaggctcaaaattccaaaatgtcatttcagcgtgtgcaccgcttcataaggccagtctcgtgaatcctacaagtttgatggtattctgggcctcgggcatagcccatttatcataaataaggacaattttgtaattctctccttttctttttgtttgcttgtatttgtatttcacatgggactcacttatgttgatgtattttagcttgtaaaaaagagtaaaaacattattcagtgaagtgacgcattgagttcatgattaagtttcaattatcctttttaataggtccatgccacgcttgtataaggattgagtaaaaaaactaactttattacaccacagtagataacaataagtcatatcagttgcatcacacaagcacactatttacttcttgtttttggatcaattctttggtgtctgtaaacccatacaacataacttgaatatgaacagcactgcagggataattgaaacctaggagtaaataaaatggatgtgaacgtttcagattatttggcattctgtaaaagacccaacacccaaatgcttgaaaaaatatatcatgttgtaaaaattcgatctgcatttccttctgtaagcaataaaacatggctcttttaagaattaccttgacaaaaaggtaaaatataatatcaataataactccggataggaaaaacttattcttctgcttataattatatcttaaatttaagtctatgacaatgtgaaatatttattgctagtgctacattttaatgtgtaatgaaagcactttcttttggacattcgaattctcctttgaaaatgatatactcctgtgattctggtttttaggagaattgcatgcatggcctagtggtagcgttgagataatgagttaagctttttgaagtttctaatgtgaattgtttttcttttaacttaaatactcattcagcgaaagatcctctagcgtaatgggtaaggattccgagtatcaccttcaggttctgggttcgaccctcatcgggggcgaatttcgggcttggttaaaaaaaatactctcactatgccccgcccgctcccgggttacgttctgtgcgccaccctctggctgagctgttgtaaagtgggcgatgacggcccgctagtgatggggggcaggggtggacccaagtacggctgagtgagggcacatgcccccgcccatttctttgttctaagtactagggtctaaattttcaccgtgagcccccctgcttagtctaatttagatgcccttgctctggtattttgatgctcgtccttacaagtatgcccttagtcatattttgtcctgggtccgcccttgatgagaggggccagggtttagagattttctcggccgggaccaatgttccggtctcttcttaatatagtaccgggacagtctttccctctccggccgagttttttatactcactctaggtgcaaacgtaagttgttcaggcttgtgcttaaggattaagaaagtaaataaaatgaccatgttacactttttttatttataccgtatcgggaaagataaaccatttatctgtgagtggtagcatggtcaaggagaaaaaaatagatgcatagaggttctgggacgactcacatttagagcatagttgaaaaggctaaaacaaccaggagggggtggggtcgggatacataaattttcatgagtgatggttgaactattgagggtgcaaaataattttttcaacaaataatgtggtgaaatacctaagaggggtgcacctagcatagattttttggggctccccttggcctctcctttcttccgccctgaaaacaacctacatggatacatctgcaaccagagggagtatctgatgctttttcctgggcagggagagctatgaggcgtatgtcctcaaagccactttgcattgtgtgaaaccaatatcgatctttgttacttcatcatgcgtgaacatttgtggaaactactagcttacaagcattagtgacagctcagaaaaaagttatctctgaaaggtttcatgtgtaccgtgggaaatgagaaatgttgccaactcaaacaccttcaatatgttgtttgcaggcaaactcttctggaagaaaggtgtctaaaactatgaacgggttacagaaaggtataaaccacggctgtgcattttggaagtatcatctatagatgtctgttgaggggaaagccgtacgccaacgttatttactcagaaacagcttcaacacacagttgtctgctttatgatggcatctccacccaggcacccaccatcacctatctctcgtgcctgtttattttcttgccctttctgatcataaaaaatcattaagagtttgcaaacatgcataggcatatcaatatgctcatttattaatttgctagcagatcatcttcctactctttactttatttattgtttgaaaaatatgtcctgcacctagggagctcgtatacagtaccaatgcatcttcattaaatgtgaatttcagaaaggaagtaggaacctatgagagtatttttcaaaattaattagcggcttctattatgtttatagcaaaggccaagggcaaaattggaacactaatgatggttggttgcatgagtctgtcgattacttgcaagaaatgtgaacctttgtttctgtgcgtgggcataaaacaaacagcttctagcctcttttacggtacttgcacttgcaagaaatgtgaactccttttcatttctgtatgtggacataatgccaaagcatccaggctttttcatggttgttgatgtctttacacagttcatctccaccagtatgccctctcatactctatataaacacatcaacagcatcgcaattagccacaagatcacttcgggaggcaagtgcgatttcgatctcgcagccaccttttttttgttctgttgtaagtatactttcccttaccatctttatctgttagtttaatttgtaattgggaagtattagtggaaagaggatgagatgctatcatctatgtactctgcaaatgcatctgacgttatatgggctgcttcatataatttgaattgctccattcttgccgacaatatattgcaaggtatatgcctagttccatcaaaagttctgttttttcattctaaaagcattttagtggcacgcattttttgtccatgagggaaaggaaatctgttttggttactttgcttgaggtgcattcttcatatgtccagttttatggaagtaataaacttcagtttggtcataagatgtcatattaaagggcaaacatatattcaatgttcaattcatcgtaaatgttccctttttgtaaaagattgcatactcatttatttgagttgcaggtgtatctagtagttggaggagatatgcagtttgcacttgcattggacacgaactcaggtcctcaccagataagatcttgtgagggtgatgggattgacaggttggaaaaattaagtattgggggcagaaagcaggagaaagctttgagaaataggtgctttggtggtagagttgctgcaactacacaatgtattcttacctcagatgcttgtcctgaaactcttgtaagtatccacctcaattattactcttacatgttggtttactttacgtttgtcttttcaagggaaatttactgtattttttgggttttgtgggagttctatacttctgttggactggttattgtaaagatttgttcaaatagggtcatctaataattgtttgaaatctgggaactgtggtttcactgcgttcaggaaaaagtgaattcttggttactgcatgaataacttatggaaatagaccttagagttgctgcatgattatcacaaatcattgctacgatatcttataatagttctttcgacctcgcattacatatataactgcaactcgtagttgcgttcaaaaaaaaatgcaactcttagaacgctcaccagtgtaatctttcctgaattgttatttaatggcatgtatgcactacttgtatacttatctaggattaagtaatctaactctaggccccatatttgcagcattctcaaacacagtcctctaggaaaaattatgctgatgcaaaccgtgtatctgctatcattttgggcggaggcaaaggatctgggctctttcctctgacaagcacaagagctacgcctgctgtaagggataacactgaacatccaacgttgattactctattatagtattatacagactgtacttttcgaatttatcttagttttctacaatatttagtggattcttctcattttcaagatacacaattgatccataatcgaagtggtatgtaagacagtgagttaaaagattatattttttgggagacttccagtcaaattttcttagaagtttttttggtccagatgttcataaagtcgccgctttcatactttttttaattttttaattggtgcactattaggtacctgttggaggatgttacaggcttattgatatccctatgagtaactgcttcaacagtggtataaataagatatttgtgatgagtcagttcaattctacttcgcttaaccgccatattcatcgtacataccttgaaggcgggatcaactttgctgatggatctgtacaggtgatttacctcatcttgttgatgtgtaatactgtaattaggagtagatttgtgtggagagaataataaacagatgccgagattcttctctaaaagtctagatccaaaggcattgtggttcaaaacactatggacttctaccatttatgtcattactttgccttaatgttccattgaatggggcaaattattgattctacaagtgtttaattaaaaactaattgttcatcctgcaggtattagcggctacacaaatgcctgaagagccagctggatggttccagggtacagcagactctatcagaaaatttatctgggtactcgaggtagttgatattttctcgtttatgaatgtccattcactcattcctgtagcattgtttctttgtaattttgagttctcctgtatttctttaggattattacagtcacaaatccattgacaacattgtaatcttgagtggcgatcagctttatcggatgaattacatggaacttgtgcaggtatggtgttctcttgttcctcatgtttcacgtaatgtcctgattttggattaaccaactacttttggcatgcattatttccagaaacatgtcgaggacgatgctgatatcactatatcatgtgctcctgttgatgagaggtaatcagttgtttatatcatcctaatatgaatatgtcatcttgttatccaacacaggatgcatatggtctaatctgctttcctttttttcccttcggaagccgagcttctaaaaatgggctagtgaagattgatcatactggacgtgtacttcaattctttgaaaaaccaaagggtgctgatttgaattctatggttagaaattccttgtgtaatccaattcttttgttttcctttctttcttgagatgaacccctcttttagttatttccatggataacctgtacttgacttattcagaaatgattttctattttgctgtagaatctgacactaaagctaatagctactgatgttgcagagagttgagaccaacttcctgagctatgctatagatgatgcacagaaatatccataccttgcatcaatgggcatttatgtcttcaagaaagatgcacttttagaccttctcaagtaatcactttcctgtgacttttttctatccaactcctagtttaccttctaacagtgtcaattcttaggtcaaaatatattcaattacatgactttggatctgaaatcctcccaagagctgtactagatcatagtgtgcaggtaagtctgatctgtctggagtatgtgttctgtaaactgtaaattcttcatgtcaaaaagttgtttttgtttccagtttccactagtttttatttaccaatgcacgatttatgtattttcgcttccatgcatcatacatactaacaatacattttacgtattgtgttaggcatgcatttttacgggctattgggaggatgttggaacaatcaaatcattctttgatgcaaacttggccctcactgagcaggtactctgtcatgtattctgtactgcatatatattacctggaattcaatgcatagaatgtgttagaccatcttagttccatcctgttttcttcaattagcttatcatttaatagttgttgactagaatctaaacacaaatttacctaatatgtttctctcttcagccttccaagtttgatttttatgatccaaaaacacctttcttcactgcaccccgatgcttgcctccgacgcaattggacaagtgcaaggtatatgtcttaccgagcacaattgttacctgagcaagattttgtgtacttgacttgttctcctccacagatgaaagatgcatttatctcagatggttgcttactgagagaatgcaacatcgagcattctgtgattggagtctgctcacgtgtcagctctggatgtgaactcaaggtacatactctgccaatgtatatgctgatgttttatacattctcttgcataatttgattcgagtcaccacaattagtgtaactgcaatctactcttgagtataccatttcaacaccaggcatcaccaaatcacacagaacaatagcaacgaagccttttagttccaagaaatttagggtagcctagagttgaaatctaacaaaacaaaagtcaaagctctatcacgtggatagttgttttccatgcactcttatttaagctaaatttttgggtatactacatccatttaattattgttttattgcttcttccctttgcctttcccccattactatcgcgtcttaagatcatactacgcactagtgtctttagaggtctctggtggacatgttcaaaccatctcaatcgaatcggtgttggacaagtttttcttgaatttgtgctacacctaacctatcacgtatatcatcgtttcaaacttgatccttcttgtatcatcataaatccaatgcaacatacgcatttctgcaacatttatctgttgaacatgtcatctttttgtaggttaacattatgcaccatacaatgtagcatgtctaatcatcatcctataaaatttacattttagcttatgtggtatcctcttgccacttagaacatcatatgtttgatgccatttcatccaccctgctttgattctatggctaacatcttcattaatatcctcgcctctctgtatcattggtcctaaatatggaaatacattctttctgggcactacttgaccttccaaactaacgtctcctttgatcctttcttgtgtgtagtagtaccgaagtcacatctcatatattcggttttagttctactaagtcccgggttcgatccccctcaggggtaaatttcgggcttggtaaaaaaaatcccctcgctgtgtcccgcccgctctcggggatcgatatcctgcgcgccaccctccggctgggcattgcagagtgggcagttgatcggctcgttagtgatggggagcggggttcaagggttttctcggccgggaccatgtttcggtctcttaatataataccgggagggcagtctttccctccccggtcgagttttagttctaccgagtctaaaacctttggactctagagtcccctgtcacaactcacaactctagttttctatttacttctacctagcgtttattaatgatcactatatcgtctgtaaaaagcatacaccaaggtaatccccttgtatgtcccttgtaatattatccatcacaagaaaaaaaaggtaaggctcaaagttgacttttgatataatcctattctaatcgagaagtcatctgtatcttcgtctcttgttcgaacactagtcacaattttttttgtacatgttcttaatgagtccaacgtaatattccttgatattttgtaagccctcatcaagtcaatgaaaatcacgtgtaggtccttcatttgttccttatactgctccatcacttgtctcattaagaaaatatctctcatagttaaccttttggcatgaaacaaaatcacacagaagttgtttcctttttttaagatcccacacaaaagaggtttgatctaaggaatctggatccctgacaggtttatcaaaatcctttgtgtttttcttaaaactgaatattcctccagcttctagtattgatgtaatattcaatctgtttagcaagtgaacaccttggttcttgttgttactgtacatcccacccacccccccgaggcccagattaccacgacatgaatacaagaatattgaacccagatctagagtttgtttgtactgttgaaaatcggtgacaattcattttgttattgcgctttctgataacgacaggactccgtgatgatgggagcggacacctatgaaactgaagaagaagcttcaaagctactgttagctgggaaggtcccagttggaataggaaggaacacaaagataaggtgagtatggatgtggaaccaccggttagttcccaaaaatatcactcactgatacctgatggtatcctctgattattttcaggaactgtatcattgacatgaatgctaggattgggaagaacgtggtgatcacaaacagtaaggtgagcgagcgcacctacatgggtgcagaatcttgtgtgctcatctatcctaattcggtaattcctatccagcgctagtcttgtgaccatggggcatgggttcgactctgtgacagggcatccaagaggctgatcacccggaagaagggtactacataaggtctggaatcgtggtgatcttgaagaatgcaaccatcaacgatgggtctgtcatatagatcggctgcgtttgcgtctacaaaacaagaacctacaatggtattgcatcgatggatcgtgtaaccttggtatggtaagagccgcttgacaggaagtcgagcgttcgggcgcaagatgcgttgtctggcatgctgttccttgaccatttgtgctgctagtatgtactgttataagctgccctagaagttgcagcaaacctttttatgaacctttgtatttccattacctgctttggatcaactatatctgtcatcctatatattactaaatttttacgtgtttttctaa

SEQ ID NO: 6 - protein, 672 aa, Zea mays
  MISC_FEATURE (1)..(672): modified sh2 protein
  MISC_FEATURE (249)..(249): T249K
  MISC_FEATURE (252)..(252): Q252G
MGGQCCELEQCGNVGGSGDTLRERKEAGTSDYLAFCKRPNTQMLEKIYHVVKIRSAFPSANSSGRKVSKTMNGLQKAKAKGKIGTLMMVGCMSLSITCKKCEPLFLSSPPVCPLILYINTSTASQLATRSLREASAISISQPPFFCSVVYLVVGGDMQFALALDTNSGPHQIRSCEGDGIDRLEKLSIGGRKQEKALRNRCFGGRVAATTQCILTSDACPETLHSQTQSSRKNYADANRVSAIILGGGKGSGLFPLTSTRATPAVPVGGCYRLIDIPMSNCFNSGINKIFVMSQFNSTSLNRHIHRTYLEGGINFADGSVQVLAATQMPEEPAGWFQGTADSIRKFIWVLEDYYSHKSIDNIVILSGDQLYRMNYMELVQKHVEDDADITISCAPVDESRASKNGLVKIDHTGRVLQFFEKPKGADLNSMRVETNFLSYAIDDAQKYPYLASMGIYVFKKDALLDLLKSKYIQLHDFGSEILPRAVLDHSVQACIFTGYWEDVGTIKSFFDANLALTEQPSKFDFYDPKTPFFTAPRCLPPTQLDKCKMKDAFISDGCLLRECNIEHSVIGVCSRVSSGCELKDSVMMGADTYETEEEASKLLLAGKVPVGIGRNTKIRNCIIDMNARIGKNVVITNSKGIQEADHPEEGYYIRSGIVVILKNATINDGSVI

SEQ ID NO: 7 - DNA, 9365 bp, Zea mays
  misc_feature (1)..(9365): modified sh2 genomic DNA
  misc_feature (4756)..(4758): G312N mutation
  misc_feature (4771)..(4773): D317G mutation
atgggcgggcagtgctgtgagttagagcagtgtggcaatgttggtggtagtggtgacacccttcgggagcggaaggaggctggaacttcaggtcagttctattgccataaataaataaaataaaataaatgaggctcaaaattccaaaatgtcatttcagcgtgtgcaccgcttcataaggccagtctcgtgaatcctacaagtttgatggtattctgggcctcgggcatagcccatttatcataaataaggacaattttgtaattctctccttttctttttgtttgcttgtatttgtatttcacatgggactcacttatgttgatgtattttagcttgtaaaaaagagtaaaaacattattcagtgaagtgacgcattgagttcatgattaagtttcaattatcctttttaataggtccatgccacgcttgtataaggattgagtaaaaaaactaactttattacaccacagtagataacaataagtcatatcagttgcatcacacaagcacactatttacttcttgtttttggatcaattctttggtgtctgtaaacccatacaacataacttgaatatgaacagcactgcagggataattgaaacctaggagtaaataaaatggatgtgaacgtttcagattatttggcattctgtaaaagacccaacacccaaatgcttgaaaaaatatatcatgttgtaaaaattcgatctgcatttccttctgtaagcaataaaacatggctcttttaagaattaccttgacaaaaaggtaaaatataatatcaataataactccggataggaaaaacttattcttctgcttataattatatcttaaatttaagtctatgacaatgtgaaatatttattgctagtgctacattttaatgtgtaatgaaagcactttcttttggacattcgaattctcctttgaaaatgatatactcctgtgattctggtttttaggagaattgcatgcatggcctagtggtagcgttgagataatgagttaagctttttgaagtttctaatgtgaattgtttttcttttaacttaaatactcattcagcgaaagatcctctagcgtaatgggtaaggattccgagtatcaccttcaggttctgggttcgaccctcatcgggggcgaatttcgggcttggttaaaaaaaatactctcactatgccccgcccgctcccgggttacgttctgtgcgccaccctctggctgagctgttgtaaagtgggcgatgacggcccgctagtgatggggggcaggggtggacccaagtacggctgagtgagggcacatgcccccgcccatttctttgttctaagtactagggtctaaattttcaccgtgagcccccctgcttagtctaatttagatgcccttgctctggtattttgatgctcgtccttacaagtatgcccttagtcatattttgtcctgggtccgcccttgatgagaggggccagggtttagagattttctcggccgggaccaatgttccggtctcttcttaatatagtaccgggacagtctttccctctccggccgagttttttatactcactctaggtgcaaacgtaagttgttcaggcttgtgcttaaggattaagaaagtaaataaaatgaccatgttacactttttttatttataccgtatcgggaaagataaaccatttatctgtgagtggtagcatggtcaaggagaaaaaaatagatgcatagaggttctgggacgactcacatttagagcatagttgaaaaggctaaaacaaccaggagggggtggggtcgggatacataaattttcatgagtgatggttgaactattgagggtgcaaaataattttttcaacaaataatgtggtgaaatacctaagaggggtgcacctagcatagattttttggggctccccttggcctctcctttcttccgccctgaaaacaacctacatggatacatctgcaaccagagggagtatctgatgctttttcctgggcagggagagctatgaggcgtatgtcctcaaagccactttgcattgtgtgaaaccaatatcgatctttgttacttcatcatgcgtgaacatttgtggaaactactagcttacaagcattagtgacagctcagaaaaaagttatctctgaaaggtttcatgtgtaccgtgggaaatgagaaatgttgccaactcaaacaccttcaatatgttgtttgcaggcaaactcttctggaagaaaggtgtctaaaactatgaacgggttacagaaaggtataaaccacggctgtgcattttggaagtatcatctatagatgtctgttgaggggaaagccgtacgccaacgttatttactcagaaacagcttcaacacacagttgtctgctttatgatggcatctccacccaggcacccaccatcacctatctctcgtgcctgtttattttcttgccctttctgatcataaaaaatcattaagagtttgcaaacatgcataggcatatcaatatgctcatttattaatttgctagcagatcatcttcctactctttactttatttattgtttgaaaaatatgtcctgcacctagggagctcgtatacagtaccaatgcatcttcattaaatgtgaatttcagaaaggaagtaggaacctatgagagtatttttcaaaattaattagcggcttctattatgtttatagcaaaggccaagggcaaaattggaacactaatgatggttggttgcatgagtctgtcgattacttgcaagaaatgtgaacctttgtttctgtgcgtgggcataaaacaaacagcttctagcctcttttacggtacttgcacttgcaagaaatgtgaactccttttcatttctgtatgtggacataatgccaaagcatccaggctttttcatggttgttgatgtctttacacagttcatctccaccagtatgccctctcatactctatataaacacatcaacagcatcgcaattagccacaagatcacttcgggaggcaagtgcgatttcgatctcgcagccaccttttttttgttctgttgtaagtatactttcccttaccatctttatctgttagtttaatttgtaattgggaagtattagtggaaagaggatgagatgctatcatctatgtactctgcaaatgcatctgacgttatatgggctgcttcatataatttgaattgctccattcttgccgacaatatattgcaaggtatatgcctagttccatcaaaagttctgttttttcattctaaaagcattttagtggcacgcattttttgtccatgagggaaaggaaatctgttttggttactttgcttgaggtgcattcttcatatgtccagttttatggaagtaataaacttcagtttggtcataagatgtcatattaaagggcaaacatatattcaatgttcaattcatcgtaaatgttccctttttgtaaaagattgcatactcatttatttgagttgcaggtgtatctagtagttggaggagatatgcagtttgcacttgcattggacacgaactcaggtcctcaccagataagatcttgtgagggtgatgggattgacaggttggaaaaattaagtattgggggcagaaagcaggagaaagctttgagaaataggtgctttggtggtagagttgctgcaactacacaatgtattcttacctcagatgcttgtcctgaaactcttgtaagtatccacctcaattattactcttacatgttggtttactttacgtttgtcttttcaagggaaatttactgtattttttgggttttgtgggagttctatacttctgttggactggttattgtaaagatttgttcaaatagggtcatctaataattgtttgaaatctgggaactgtggtttcactgcgttcaggaaaaagtgaattcttggttactgcatgaataacttatggaaatagaccttagagttgctgcatgattatcacaaatcattgctacgatatcttataatagttctttcgacctcgcattacatatataactgcaactcgtagttgcgttcaaaaaaaaatgcaactcttagaacgctcaccagtgtaatctttcctgaattgttatttaatggcatgtatgcactacttgtatacttatctaggattaagtaatctaactctaggccccatatttgcagcattctcaaacacagtcctctaggaaaaattatgctgatgcaaaccgtgtatctgctatcattttgggcggaggcactggatctcagctctttcctctgacaagcacaagagctacgcctgctgtaagggataacactgaacatccaacgttgattactctattatagtattatacagactgtacttttcgaatttatcttagttttctacaatatttagtggattcttctcattttcaagatacacaattgatccataatcgaagtggtatgtaagacagtgagttaaaagattatattttttgggagacttccagtcaaattttcttagaagtttttttggtccagatgttcataaagtcgccgctttcatactttttttaattttttaattggtgcactattaggtacctgttggaggatgttacaggcttattgatatccctatgagtaactgcttcaacagtggtataaataagatatttgtgatgagtcagttcaattctacttcgcttaaccgccatattcatcgtacataccttgaaggcaacatcaactttgctggtggatctgtacaggtgatttacctcatcttgttgatgtgtaatactgtaattaggagtagatttgtgtggagagaataataaacagatgccgagattcttctctaaaagtctagatccaaaggcattgtggttcaaaacactatggacttctaccatttatgtcattactttgccttaatgttccattgaatggggcaaattattgattctacaagtgtttaattaaaaactaattgttcatcctgcaggtattagcggctacacaaatgcctgaagagccagctggatggttccagggtacagcagactctatcagaaaatttatctgggtactcgaggtagttgatattttctcgtttatgaatgtccattcactcattcctgtagcattgtttctttgtaattttgagttctcctgtatttctttaggattattacagtcacaaatccattgacaacattgtaatcttgagtggcgatcagctttatcggatgaattacatggaacttgtgcaggtatggtgttctcttgttcctcatgtttcacgtaatgtcctgattttggattaaccaactacttttggcatgcattatttccagaaacatgtcgaggacgatgctgatatcactatatcatgtgctcctgttgatgagaggtaatcagttgtttatatcatcctaatatgaatatgtcatcttgttatccaacacaggatgcatatggtctaatctgctttcctttttttcccttcggaagccgagcttctaaaaatgggctagtgaagattgatcatactggacgtgtacttcaattctttgaaaaaccaaagggtgctgatttgaattctatggttagaaattccttgtgtaatccaattcttttgttttcctttctttcttgagatgaacccctcttttagttatttccatggataacctgtacttgacttattcagaaatgattttctattttgctgtagaatctgacactaaagctaatagctactgatgttgcagagagttgagaccaacttcctgagctatgctatagatgatgcacagaaatatccataccttgcatcaatgggcatttatgtcttcaagaaagatgcacttttagaccttctcaagtaatcactttcctgtgacttttttctatccaactcctagtttaccttctaacagtgtcaattcttaggtcaaaatatattcaattacatgactttggatctgaaatcctcccaagagctgtactagatcatagtgtgcaggtaagtctgatctgtctggagtatgtgttctgtaaactgtaaattcttcatgtcaaaaagttgtttttgtttccagtttccactagtttttatttaccaatgcacgatttatgtattttcgcttccatgcatcatacatactaacaatacattttacgtattgtgttaggcatgcatttttacgggctattgggaggatgttggaacaatcaaatcattctttgatgcaaacttggccctcactgagcaggtactctgtcatgtattctgtactgcatatatattacctggaattcaatgcatagaatgtgttagaccatcttagttccatcctgttttcttcaattagcttatcatttaatagttgttgactagaatctaaacacaaatttacctaatatgtttctctcttcagccttccaagtttgatttttatgatccaaaaacacctttcttcactgcaccccgatgcttgcctccgacgcaattggacaagtgcaaggtatatgtcttaccgagcacaattgttacctgagcaagattttgtgtacttgacttgttctcctccacagatgaaagatgcatttatctcagatggttgcttactgagagaatgcaacatcgagcattctgtgattggagtctgctcacgtgtcagctctggatgtgaactcaaggtacatactctgccaatgtatatgctgatgttttatacattctcttgcataatttgattcgagtcaccacaattagtgtaactgcaatctactcttgagtataccatttcaacaccaggcatcaccaaatcacacagaacaatagcaacgaagccttttagttccaagaaatttagggtagcctagagttgaaatctaacaaaacaaaagtcaaagctctatcacgtggatagttgttttccatgcactcttatttaagctaaatttttgggtatactacatccatttaattattgttttattgcttcttccctttgcctttcccccattactatcgcgtcttaagatcatactacgcactagtgtctttagaggtctctggtggacatgttcaaaccatctcaatcgaatcggtgttggacaagtttttcttgaatttgtgctacacctaacctatcacgtatatcatcgtttcaaacttgatccttcttgtatcatcataaatccaatgcaacatacgcatttctgcaacatttatctgttgaacatgtcatctttttgtaggttaacattatgcaccatacaatgtagcatgtctaatcatcatcctataaaatttacattttagcttatgtggtatcctcttgccacttagaacatcatatgtttgatgccatttcatccaccctgctttgattctatggctaacatcttcattaatatcctcgcctctctgtatcattggtcctaaatatggaaatacattctttctgggcactacttgaccttccaaactaacgtctcctttgatcctttcttgtgtgtagtagtaccgaagtcacatctcatatattcggttttagttctactaagtcccgggttcgatccccctcaggggtaaatttcgggcttggtaaaaaaaatcccctcgctgtgtcccgcccgctctcggggatcgatatcctgcgcgccaccctccggctgggcattgcagagtgggcagttgatcggctcgttagtgatggggagcggggttcaagggttttctcggccgggaccatgtttcggtctcttaatataataccgggagggcagtctttccctccccggtcgagttttagttctaccgagtctaaaacctttggactctagagtcccctgtcacaactcacaactctagttttctatttacttctacctagcgtttattaatgatcactatatcgtctgtaaaaagcatacaccaaggtaatccccttgtatgtcccttgtaatattatccatcacaagaaaaaaaaggtaaggctcaaagttgacttttgatataatcctattctaatcgagaagtcatctgtatcttcgtctcttgttcgaacactagtcacaattttttttgtacatgttcttaatgagtccaacgtaatattccttgatattttgtaagccctcatcaagtcaatgaaaatcacgtgtaggtccttcatttgttccttatactgctccatcacttgtctcattaagaaaatatctctcatagttaaccttttggcatgaaacaaaatcacacagaagttgtttcctttttttaagatcccacacaaaagaggtttgatctaaggaatctggatccctgacaggtttatcaaaatcctttgtgtttttcttaaaactgaatattcctccagcttctagtattgatgtaatattcaatctgtttagcaagtgaacaccttggttcttgttgttactgtacatcccacccacccccccgaggcccagattaccacgacatgaatacaagaatattgaacccagatctagagtttgtttgtactgttgaaaatcggtgacaattcattttgttattgcgctttctgataacgacaggactccgtgatgatgggagcggacacctatgaaactgaagaagaagcttcaaagctactgttagctgggaaggtcccagttggaataggaaggaacacaaagataaggtgagtatggatgtggaaccaccggttagttcccaaaaatatcactcactgatacctgatggtatcctctgattattttcaggaactgtatcattgacatgaatgctaggattgggaagaacgtggtgatcacaaacagtaaggtgagcgagcgcacctacatgggtgcagaatcttgtgtgctcatctatcctaattcggtaattcctatccagcgctagtcttgtgaccatggggcatgggttcgactctgtgacagggcatccaagaggctgatcacccggaagaagggtactacataaggtctggaatcgtggtgatcttgaagaatgcaaccatcaacgatgggtctgtcatatagatcggctgcgtttgcgtctacaaaacaagaacctacaatggtattgcatcgatggatcgtgtaaccttggtatggtaagagccgcttgacaggaagtcgagcgttcgggcgcaagatgcgttgtctggcatgctgttccttgaccatttgtgctgctagtatgtactgttataagctgccctagaagttgcagcaaacctttttatgaacctttgtatttccattacctgctttggatcaactatatctgtcatcctatatattactaaatttttacgtgtttttctaa

SEQ ID NO: 8 - protein, 672 aa, Zea mays
  MISC_FEATURE (1)..(672): modified sh2 protein
  MISC_FEATURE (312)..(312): G312N
  MISC_FEATURE (317)..(317): D317G
MGGQCCELEQCGNVGGSGDTLRERKEAGTSDYLAFCKRPNTQMLEKIYHVVKIRSAFPSANSSGRKVSKTMNGLQKAKAKGKIGTLMMVGCMSLSITCKKCEPLFLSSPPVCPLILYINTSTASQLATRSLREASAISISQPPFFCSVVYLVVGGDMQFALALDTNSGPHQIRSCEGDGIDRLEKLSIGGRKQEKALRNRCFGGRVAATTQCILTSDACPETLHSQTQSSRKNYADANRVSAIILGGGTGSQLFPLTSTRATPAVPVGGCYRLIDIPMSNCFNSGINKIFVMSQFNSTSLNRHIHRTYLEGNINFAGGSVQVLAATQMPEEPAGWFQGTADSIRKFIWVLEDYYSHKSIDNIVILSGDQLYRMNYMELVQKHVEDDADITISCAPVDESRASKNGLVKIDHTGRVLQFFEKPKGADLNSMRVETNFLSYAIDDAQKYPYLASMGIYVFKKDALLDLLKSKYIQLHDFGSEILPRAVLDHSVQACIFTGYWEDVGTIKSFFDANLALTEQPSKFDFYDPKTPFFTAPRCLPPTQLDKCKMKDAFISDGCLLRECNIEHSVIGVCSRVSSGCELKDSVMMGADTYETEEEASKLLLAGKVPVGIGRNTKIRNCIIDMNARIGKNVVITNSKGIQEADHPEEGYYIRSGIVVILKNATINDGSVI

SEQ ID NO: 9 - DNA, 9365 bp, Zea mays
  misc_feature (1)..(9365): modified sh2 genomic DNA
  misc_feature (4283)..(4285): T249K modification
  misc_feature (4292)..(4294): Q252G modification
  misc_feature (4756)..(4758): G312N modification
  misc_feature (4771)..(4773): D317G modification
atgggcgggcagtgctgtgagttagagcagtgtggcaatgttggtggtagtggtgacacccttcgggagcggaaggaggctggaacttcaggtcagttctattgccataaataaataaaataaaataaatgaggctcaaaattccaaaatgtcatttcagcgtgtgcaccgcttcataaggccagtctcgtgaatcctacaagtttgatggtattctgggcctcgggcatagcccatttatcataaataaggacaattttgtaattctctccttttctttttgtttgcttgtatttgtatttcacatgggactcacttatgttgatgtattttagcttgtaaaaaagagtaaaaacattattcagtgaagtgacgcattgagttcatgattaagtttcaattatcctttttaataggtccatgccacgcttgtataaggattgagtaaaaaaactaactttattacaccacagtagataacaataagtcatatcagttgcatcacacaagcacactatttacttcttgtttttggatcaattctttggtgtctgtaaacccatacaacataacttgaatatgaacagcactgcagggataattgaaacctaggagtaaataaaatggatgtgaacgtttcagattatttggcattctgtaaaagacccaacacccaaatgcttgaaaaaatatatcatgttgtaaaaattcgatctgcatttccttctgtaagcaataaaacatggctcttttaagaattaccttgacaaaaaggtaaaatataatatcaataataactccggataggaaaaacttattcttctgcttataattatatcttaaatttaagtctatgacaatgtgaaatatttattgctagtgctacattttaatgtgtaatgaaagcactttcttttggacattcgaattctcctttgaaaatgatatactcctgtgattctggtttttaggagaattgcatgcatggcctagtggtagcgttgagataatgagttaagctttttgaagtttctaatgtgaattgtttttcttttaacttaaatactcattcagcgaaagatcctctagcgtaatgggtaaggattccgagtatcaccttcaggttctgggttcgaccctcatcgggggcgaatttcgggcttggttaaaaaaaatactctcactatgccccgcccgctcccgggttacgttctgtgcgccaccctctggctgagctgttgtaaagtgggcgatgacggcccgctagtgatggggggcaggggtggacccaagtacggctgagtgagggcacatgcccccgcccatttctttgttctaagtactagggtctaaattttcaccgtgagcccccctgcttagtctaatttagatgcccttgctctggtattttgatgctcgtccttacaagtatgcccttagtcatattttgtcctgggtccgcccttgatgagaggggccagggtttagagattttctcggccgggaccaatgttccggtctcttcttaatatagtaccgggacagtctttccctctccggccgagttttttatactcactctaggtgcaaacgtaagttgttcaggcttgtgcttaaggattaagaaagtaaataaaatgaccatgttacactttttttatttataccgtatcgggaaagataaaccatttatctgtgagtggtagcatggtcaaggagaaaaaaatagatgcatagaggttctgggacgactcacatttagagcatagttgaaaaggctaaaacaaccaggagggggtggggtcgggatacataaattttcatgagtgatggttgaactattgagggtgcaaaataattttttcaacaaataatgtggtgaaatacctaagaggggtgcacctagcatagattttttggggctccccttggcctctcctttcttccgccctgaaaacaacctacatggatacatctgcaaccagagggagtatctgatgctttttcctgggcagggagagctatgaggcgtatgtcctcaaagccactttgcattgtgtgaaaccaatatcgatctttgttacttcatcatgcgtgaacatttgtggaaactactagcttacaagcattagtgacagctcagaaaaaagttatctctgaaaggtttcatgtgtaccgtgggaaatgagaaatgttgccaactcaaacaccttcaatatgttgtttgcaggcaaactcttctggaagaaaggtgtctaaaactatgaacgggttacagaaaggtataaaccacggctgtgcattttggaagtatcatctatagatgtctgttgaggggaaagccgtacgccaacgttatttactcagaaacagcttcaacacacagttgtctgctttatgatggcatctccacccaggcacccaccatcacctatctctcgtgcctgtttattttcttgccctttctgatcataaaaaatcattaagagtttgcaaacatgcataggcatatcaatatgctcatttattaatttgctagcagatcatcttcctactctttactttatttattgtttgaaaaatatgtcctgcacctagggagctcgtatacagtaccaatgcatcttcattaaatgtgaatttcagaaaggaagtaggaacctatgagagtatttttcaaaattaattagcggcttctattatgtttatagcaaaggccaagggcaaaattggaacactaatgatggttggttgcatgagtctgtcgattacttgcaagaaatgtgaacctttgtttctgtgcgtgggcataaaacaaacagcttctagcctcttttacggtacttgcacttgcaagaaatgtgaactccttttcatttctgtatgtggacataatgccaaagcatccaggctttttcatggttgttgatgtctttacacagttcatctccaccagtatgccctctcatactctatataaacacatcaacagcatcgcaattagccacaagatcacttcgggaggcaagtgcgatttcgatctcgcagccaccttttttttgttctgttgtaagtatactttcccttaccatctttatctgttagtttaatttgtaattgggaagtattagtggaaagaggatgagatgctatcatctatgtactctgcaaatgcatctgacgttatatgggctgcttcatataatttgaattgctccattcttgccgacaatatattgcaaggtatatgcctagttccatcaaaagttctgttttttcattctaaaagcattttagtggcacgcattttttgtccatgagggaaaggaaatctgttttggttactttgcttgaggtgcattcttcatatgtccagttttatggaagtaataaacttcagtttggtcataagatgtcatattaaagggcaaacatatattcaatgttcaattcatcgtaaatgttccctttttgtaaaagattgcatactcatttatttgagttgcaggtgtatctagtagttggaggagatatgcagtttgcacttgcattggacacgaactcaggtcctcaccagataagatcttgtgagggtgatgggattgacaggttggaaaaattaagtattgggggcagaaagcaggagaaagctttgagaaataggtgctttggtggtagagttgctgcaactacacaatgtattcttacctcagatgcttgtcctgaaactcttgtaagtatccacctcaattattactcttacatgttggtttactttacgtttgtcttttcaagggaaatttactgtattttttgggttttgtgggagttctatacttctgttggactggttattgtaaagatttgttcaaatagggtcatctaataattgtttgaaatctgggaactgtggtttcactgcgttcaggaaaaagtgaattcttggttactgcatgaataacttatggaaatagaccttagagttgctgcatgattatcacaaatcattgctacgatatcttataatagttctttcgacctcgcattacatatataactgcaactcgtagttgcgttcaaaaaaaaatgcaactcttagaacgctcaccagtgtaatctttcctgaattgttatttaatggcatgtatgcactacttgtatacttatctaggattaagtaatctaactctaggccccatatttgcagcattctcaaacacagtcctctaggaaaaattatgctgatgcaaaccgtgtatctgctatcattttgggcggaggcaaaggatctgggctctttcctctgacaagcacaagagctacgcctgctgtaagggataacactgaacatccaacgttgattactctattatagtattatacagactgtacttttcgaatttatcttagttttctacaatatttagtggattcttctcattttcaagatacacaattgatccataatcgaagtggtatgtaagacagtgagttaaaagattatattttttgggagacttccagtcaaattttcttagaagtttttttggtccagatgttcataaagtcgccgctttcatactttttttaattttttaattggtgcactattaggtacctgttggaggatgttacaggcttattgatatccctatgagtaactgcttcaacagtggtataaataagatatttgtgatgagtcagttcaattctacttcgcttaaccgccatattcatcgtacataccttgaaggcaacatcaactttgctggtggatctgtacaggtgatttacctcatcttgttgatgtgtaatactgtaattaggagtagatttgtgtggagagaataataaacagatgccgagattcttctctaaaagtctagatccaaaggcattgtggttcaaaacactatggacttctaccatttatgtcattactttgccttaatgttccattgaatggggcaaattattgattctacaagtgtttaattaaaaactaattgttcatcctgcaggtattagcggctacacaaatgcctgaagagccagctggatggttccagggtacagcagactctatcagaaaatttatctgggtactcgaggtagttgatattttctcgtttatgaatgtccattcactcattcctgtagcattgtttctttgtaattttgagttctcctgtatttctttaggattattacagtcacaaatccattgacaacattgtaatcttgagtggcgatcagctttatcggatgaattacatggaacttgtgcaggtatggtgttctcttgttcctcatgtttcacgtaatgtcctgattttggattaaccaactacttttggcatgcattatttccagaaacatgtcgaggacgatgctgatatcactatatcatgtgctcctgttgatgagaggtaatcagttgtttatatcatcctaatatgaatatgtcatcttgttatccaacacaggatgcatatggtctaatctgctttcctttttttcccttcggaagccgagcttctaaaaatgggctagtgaagattgatcatactggacgtgtacttcaattctttgaaaaaccaaagggtgctgatttgaattctatggttagaaattccttgtgtaatccaattcttttgttttcctttctttcttgagatgaacccctcttttagttatttccatggataacctgtacttgacttattcagaaatgattttctattttgctgtagaatctgacactaaagctaatagctactgatgttgcagagagttgagaccaacttcctgagctatgctatagatgatgcacagaaatatccataccttgcatcaatgggcatttatgtcttcaagaaagatgcacttttagaccttctcaagtaatcactttcctgtgacttttttctatccaactcctagtttaccttctaacagtgtcaattcttaggtcaaaatatattcaattacatgactttggatctgaaatcctcccaagagctgtactagatcatagtgtgcaggtaagtctgatctgtctggagtatgtgttctgtaaactgtaaattcttcatgtcaaaaagttgtttttgtttccagtttccactagtttttatttaccaatgcacgatttatgtattttcgcttccatgcatcatacatactaacaatacattttacgtattgtgttaggcatgcatttttacgggctattgggaggatgttggaacaatcaaatcattctttgatgcaaacttggccctcactgagcaggtactctgtcatgtattctgtactgcatatatattacctggaattcaatgcatagaatgtgttagaccatcttagttccatcctgttttcttcaattagcttatcatttaatagttgttgactagaatctaaacacaaatttacctaatatgtttctctcttcagccttccaagtttgatttttatgatccaaaaacacctttcttcactgcaccccgatgcttgcctccgacgcaattggacaagtgcaaggtatatgtcttaccgagcacaattgttacctgagcaagattttgtgtacttgacttgttctcctccacagatgaaagatgcatttatctcagatggttgcttactgagagaatgcaacatcgagcattctgtgattggagtctgctcacgtgtcagctctggatgtgaactcaaggtacatactctgccaatgtatatgctgatgttttatacattctcttgcataatttgattcgagtcaccacaattagtgtaactgcaatctactcttgagtataccatttcaacaccaggcatcaccaaatcacacagaacaatagcaacgaagccttttagttccaagaaatttagggtagcctagagttgaaatctaacaaaacaaaagtcaaagctctatcacgtggatagttgttttccatgcactcttatttaagctaaatttttgggtatactacatccatttaattattgttttattgcttcttccctttgcctttcccccattactatcgcgtcttaagatcatactacgcactagtgtctttagaggtctctggtggacatgttcaaaccatctcaatcgaatcggtgttggacaagtttttcttgaatttgtgctacacctaacctatcacgtatatcatcgtttcaaacttgatccttcttgtatcatcataaatccaatgcaacatacgcatttctgcaacatttatctgttgaacatgtcatctttttgtaggttaacattatgcaccatacaatgtagcatgtctaatcatcatcctataaaatttacattttagcttatgtggtatcctcttgccacttagaacatcatatgtttgatgccatttcatccaccctgctttgattctatggctaacatcttcattaatatcctcgcctctctgtatcattggtcctaaatatggaaatacattctttctgggcactacttgaccttccaaactaacgtctcctttgatcctttcttgtgtgtagtagtaccgaagtcacatctcatatattcggttttagttctactaagtcccgggttcgatccccctcaggggtaaatttcgggcttggtaaaaaaaatcccctcgctgtgtcccgcccgctctcggggatcgatatcctgcgcgccaccctccggctgggcattgcagagtgggcagttgatcggctcgttagtgatggggagcggggttcaagggttttctcggccgggaccatgtttcggtctcttaatataataccgggagggcagtctttccctccccggtcgagttttagttctaccgagtctaaaacctttggactctagagtcccctgtcacaactcacaactctagttttctatttacttctacctagcgtttattaatgatcactatatcgtctgtaaaaagcatacaccaaggtaatccccttgtatgtcccttgtaatattatccatcacaagaaaaaaaaggtaaggctcaaagttgacttttgatataatcctattctaatcgagaagtcatctgtatcttcgtctcttgttcgaacactagtcacaattttttttgtacatgttcttaatgagtccaacgtaatattccttgatattttgtaagccctcatcaagtcaatgaaaatcacgtgtaggtccttcatttgttccttatactgctccatcacttgtctcattaagaaaatatctctcatagttaaccttttggcatgaaacaaaatcacacagaagttgtttcctttttttaagatcccacacaaaagaggtttgatctaaggaatctggatccctgacaggtttatcaaaatcctttgtgtttttcttaaaactgaatattcctccagcttctagtattgatgtaatattcaatctgtttagcaagtgaacaccttggttcttgttgttactgtacatcccacccacccccccgaggcccagattaccacgacatgaatacaagaatattgaacccagatctagagtttgtttgtactgttgaaaatcggtgacaattcattttgttattgcgctttctgataacgacaggactccgtgatgatgggagcggacacctatgaaactgaagaagaagcttcaaagctactgttagctgggaaggtcccagttggaataggaaggaacacaaagataaggtgagtatggatgtggaaccaccggttagttcccaaaaatatcactcactgatacctgatggtatcctctgattattttcaggaactgtatcattgacatgaatgctaggattgggaagaacgtggtgatcacaaacagtaaggtgagcgagcgcacctacatgggtgcagaatcttgtgtgctcatctatcctaattcggtaattcctatccagcgctagtcttgtgaccatggggcatgggttcgactctgtgacagggcatccaagaggctgatcacccggaagaagggtactacataaggtctggaatcgtggtgatcttgaagaatgcaaccatcaacgatgggtctgtcatatagatcggctgcgtttgcgtctacaaaacaagaacctacaatggtattgcatcgatggatcgtgtaaccttggtatggtaagagccgcttgacaggaagtcgagcgttcgggcgcaagatgcgttgtctggcatgctgttccttgaccatttgtgctgctagtatgtactgttataagctgccctagaagttgcagcaaacctttttatgaacctttgtatttccattacctgctttggatcaactatatctgtcatcctatatattactaaatttttacgtgtttttctaa

SEQ ID NO: 10 - protein, 672 aa, Zea mays
  MISC_FEATURE (1)..(672): modified sh2 protein
  MISC_FEATURE (249)..(249): T249K
  MISC_FEATURE (252)..(252): Q252G
  MISC_FEATURE (312)..(312): G312N
  MISC_FEATURE (317)..(317): D317G
MGGQCCELEQCGNVGGSGDTLRERKEAGTSDYLAFCKRPNTQMLEKIYHVVKIRSAFPSANSSGRKVSKTMNGLQKAKAKGKIGTLMMVGCMSLSITCKKCEPLFLSSPPVCPLILYINTSTASQLATRSLREASAISISQPPFFCSVVYLVVGGDMQFALALDTNSGPHQIRSCEGDGIDRLEKLSIGGRKQEKALRNRCFGGRVAATTQCILTSDACPETLHSQTQSSRKNYADANRVSAIILGGGKGSGLFPLTSTRATPAVPVGGCYRLIDIPMSNCFNSGINKIFVMSQFNSTSLNRHIHRTYLEGNINFAGGSVQVLAATQMPEEPAGWFQGTADSIRKFIWVLEDYYSHKSIDNIVILSGDQLYRMNYMELVQKHVEDDADITISCAPVDESRASKNGLVKIDHTGRVLQFFEKPKGADLNSMRVETNFLSYAIDDAQKYPYLASMGIYVFKKDALLDLLKSKYIQLHDFGSEILPRAVLDHSVQACIFTGYWEDVGTIKSFFDANLALTEQPSKFDFYDPKTPFFTAPRCLPPTQLDKCKMKDAFISDGCLLRECNIEHSVIGVCSRVSSGCELKDSVMMGADTYETEEEASKLLLAGKVPVGIGRNTKIRNCIIDMNARIGKNVVITNSKGIQEADHPEEGYYIRSGIVVILKNATINDGSVI

SEQ ID NO: 11 - DNA, 9365 bp, Zea mays
  misc_feature (1)..(9365): modified sh2 genomic DNA
  misc_feature (4283)..(4285): T249K modification
  misc_feature (4292)..(4294): Q252G modification
  misc_feature (4756)..(4758): G312N modification
  misc_feature (4771)..(4773): D317G modification
  misc_feature (8654)..(8656): A599R modification
atgggcgggcagtgctgtgagttagagcagtgtggcaatgttggtggtagtggtgacacccttcgggagcggaaggaggctggaacttcaggtcagttctattgccataaataaataaaataaaataaatgaggctcaaaattccaaaatgtcatttcagcgtgtgcaccgcttcataaggccagtctcgtgaatcctacaagtttgatggtattctgggcctcgggcatagcccatttatcataaataaggacaattttgtaattctctccttttctttttgtttgcttgtatttgtatttcacatgggactcacttatgttgatgtattttagcttgtaaaaaagagtaaaaacattattcagtgaagtgacgcattgagttcatgattaagtttcaattatcctttttaataggtccatgccacgcttgtataaggattgagtaaaaaaactaactttattacaccacagtagataacaataagtcatatcagttgcatcacacaagcacactatttacttcttgtttttggatcaattctttggtgtctgtaaacccatacaacataacttgaatatgaacagcactgcagggataattgaaacctaggagtaaataaaatggatgtgaacgtttcagattatttggcattctgtaaaagacccaacacccaaatgcttgaaaaaatatatcatgttgtaaaaattcgatctgcatttccttctgtaagcaataaaacatggctcttttaagaattaccttgacaaaaaggtaaaatataatatcaataataactccggataggaaaaacttattcttctgcttataattatatcttaaatttaagtctatgacaatgtgaaatatttattgctagtgctacattttaatgtgtaatgaaagcactttcttttggacattcgaattctcctttgaaaatgatatactcctgtgattctggtttttaggagaattgcatgcatggcctagtggtagcgttgagataatgagttaagctttttgaagtttctaatgtgaattgtttttcttttaacttaaatactcattcagcgaaagatcctctagcgtaatgggtaaggattccgagtatcaccttcaggttctgggttcgaccctcatcgggggcgaatttcgggcttggttaaaaaaaatactctcactatgccccgcccgctcccgggttacgttctgtgcgccaccctctggctgagctgttgtaaagtgggcgatgacggcccgctagtgatggggggcaggggtggacccaagtacggctgagtgagggcacatgcccccgcccatttctttgttctaagtactagggtctaaattttcaccgtgagcccccctgcttagtctaatttagatgcccttgctctggtattttgatgctcgtccttacaagtatgcccttagtcatattttgtcctgggtccgcccttgatgagaggggccagggtttagagattttctcggccgggaccaatgttccggtctcttcttaatatagtaccgggacagtctttccctctccggccgagttttttatactcactctaggtgcaaacgtaagttgttcaggcttgtgcttaaggattaagaaagtaaataaaatgaccatgttacactttttttatttataccgtatcgggaaagataaaccatttatctgtgagtggtagcatggtcaaggagaaaaaaatagatgcatagaggttctgggacgactcacatttagagcatagttgaaaaggctaaaacaaccaggagggggtggggtcgggatacataaattttcatgagtgatggttgaactattgagggtgcaaaataattttttcaacaaataatgtggtgaaatacctaagaggggtgcacctagcatagattttttggggctccccttggcctctcctttcttccgccctgaaaacaacctacatggatacatctgcaaccagagggagtatctgatgctttttcctgggcagggagagctatgaggcgtatgtcctcaaagccactttgcattgtgtgaaaccaatatcgatctttgttacttcatcatgcgtgaacatttgtggaaactactagcttacaagcattagtgacagctcagaaaaaagttatctctgaaaggtttcatgtgtaccgtgggaaatgagaaatgttgccaactcaaacaccttcaatatgttgtttgcaggcaaactcttctggaagaaaggtgtctaaaactatgaacgggttacagaaaggtataaaccacggctgtgcattttggaagtatcatctatagatgtctgttgaggggaaagccgtacgccaacgttatttactcagaaacagcttcaacacacagttgtctgctttatgatggcatctccacccaggcacccaccatcacctatctctcgtgcctgtttattttcttgccctttctgatcataaaaaatcattaagagtttgcaaacatgcataggcatatcaatatgctcatttattaatttgctagcagatcatcttcctactctttactttatttattgtttgaaaaatatgtcctgcacctagggagctcgtatacagtaccaatgcatcttcattaaatgtgaatttcagaaaggaagtaggaacctatgagagtatttttcaaaattaattagcggcttctattatgtttatagcaaaggccaagggcaaaattggaacactaatgatggttggttgcatgagtctgtcgattacttgcaagaaatgtgaacctttgtttctgtgcgtgggcataaaacaaacagcttctagcctcttttacggtacttgcacttgcaagaaatgtgaactccttttcatttctgtatgtggacataatgccaaagcatccaggctttttcatggttgttgatgtctttacacagttcatctccaccagtatgccctctcatactctatataaacacatcaacagcatcgcaattagccacaagatcacttcgggaggcaagtgcgatttcgatctcgcagccaccttttttttgttctgttgtaagtatactttcccttaccatctttatctgttagtttaatttgtaattgggaagtattagtggaaagaggatgagatgctatcatctatgtactctgcaaatgcatctgacgttatatgggctgcttcatataatttgaattgctccattcttgccgacaatatattgcaaggtatatgcctagttccatcaaaagttctgttttttcattctaaaagcattttagtggcacgcattttttgtccatgagggaaaggaaatctgttttggttactttgcttgaggtgcattcttcatatgtccagttttatggaagtaataaacttcagtttggtcataagatgtcatattaaagggcaaacatatattcaatgttcaattcatcgtaaatgttccctttttgtaaaagattgcatactcatttatttgagttgcaggtgtatctagtagttggaggagatatgcagtttgcacttgcattggacacgaactcaggtcctcaccagataagatcttgtgagggtgatgggattgacaggttggaaaaattaagtattgggggcagaaagcaggagaaagctttgagaaataggtgctttggtggtagagttgctgcaactacacaatgtattcttacctcagatgcttgtcctgaaactcttgtaagtatccacctcaattattactcttacatgttggtttactttacgtttgtcttttcaagggaaatttactgtattttttgggttttgtgggagttctatacttctgttggactggttattgtaaagatttgttcaaatagggtcatctaataattgtttgaaatctgggaactgtggtttcactgcgttcaggaaaaagtgaattcttggttactgcatgaataacttatggaaatagaccttagagttgctgcatgattatcacaaatcattgctacgatatcttataatagttctttcgacctcgcattacatatataactgcaactcgtagttgcgttcaaaaaaaaatgcaactcttagaacgctcaccagtgtaatctttcctgaattgttatttaatggcatgtatgcactacttgtatacttatctaggattaagtaatctaactctaggccccatatttgcagcattctcaaacacagtcctctaggaaaaattatgctgatgcaaaccgtgtatctgctatcattttgggcggaggcaaaggatctgggctctttcctctgacaagcacaagagctacgcctgctgtaagggataacactgaacatccaacgttgattactctattatagtattatacagactgtacttttcgaatttatcttagttttctacaatatttagtggattcttctcattttcaagatacacaattgatccataatcgaagtggtatgtaagacagtgagttaaaagattatattttttgggagacttccagtcaaattttcttagaagtttttttggtccagatgttcataaagtcgccgctttcatactttttttaattttttaattggtgcactattaggtacctgttggaggatgttacaggcttattgatatccctatgagtaactgcttcaacagtggtataaataagatatttgtgatgagtcagttcaattctacttcgcttaaccgccatattcatcgtacataccttgaaggcaacatcaactttgctggtggatctgtacaggtgatttacctcatcttgttgatgtgtaatactgtaattaggagtagatttgtgtggagagaataataaacagatgccgagattcttctctaaaagtctagatccaaaggcattgtggttcaaaacactatggacttctaccatttatgtcattactttgccttaatgttccattgaatggggcaaattattgattctacaagtgtttaattaaaaactaattgttcatcctgcaggtattagcggctacacaaatgcctgaagagccagctggatggttccagggtacagcagactctatcagaaaatttatctgggtactcgaggtagttgatattttctcgtttatgaatgtccattcactcattcctgtagcattgtttctttgtaattttgagttctcctgtatttctttaggattattacagtcacaaatccattgacaacattgtaatcttgagtggcgatcagctttatcggatgaattacatggaacttgtgcaggtatggtgttctcttgttcctcatgtttcacgtaatgtcctgattttggattaaccaactacttttggcatgcattatttccagaaacatgtcgaggacgatgctgatatcactatatcatgtgctcctgttgatgagaggtaatcagttgtttatatcatcctaatatgaatatgtcatcttgttatccaacacaggatgcatatggtctaatctgctttcctttttttcccttcggaagccgagcttctaaaaatgggctagtgaagattgatcatactggacgtgtacttcaattctttgaaaaaccaaagggtgctgatttgaattctatggttagaaattccttgtgtaatccaattcttttgttttcctttctttcttgagatgaacccctcttttagttatttccatggataacctgtacttgacttattcagaaatgattttctattttgctgtagaatctgacactaaagctaatagctactgatgttgcagagagttgagaccaacttcctgagctatgctatagatgatgcacagaaatatccataccttgcatcaatgggcatttatgtcttcaagaaagatgcacttttagaccttctcaagtaatcactttcctgtgacttttttctatccaactcctagtttaccttctaacagtgtcaattcttaggtcaaaatatattcaattacatgactttggatctgaaatcctcccaagagctgtactagatcatagtgtgcaggtaagtctgatctgtctggagtatgtgttctgtaaactgtaaattcttcatgtcaaaaagttgtttttgtttccagtttccactagtttttatttaccaatgcacgatttatgtattttcgcttccatgcatcatacatactaacaatacattttacgtattgtgttaggcatgcatttttacgggctattgggaggatgttggaacaatcaaatcattctttgatgcaaacttggccctcactgagcaggtactctgtcatgtattctgtactgcatatatattacctggaattcaatgcatagaatgtgttagaccatcttagttccatcctgttttcttcaattagcttatcatttaatagttgttgactagaatctaaacacaaatttacctaatatgtttctctcttcagccttccaagtttgatttttatgatccaaaaacacctttcttcactgcaccccgatgcttgcctccgacgcaattggacaagtgcaaggtatatgtcttaccgagcacaattgttacctgagcaagattttgtgtacttgacttgttctcctccacagatgaaagatgcatttatctcagatggttgcttactgagagaatgcaacatcgagcattctgtgattggagtctgctcacgtgtcagctctggatgtgaactcaaggtacatactctgccaatgtatatgctgatgttttatacattctcttgcataatttgattcgagtcaccacaattagtgtaactgcaatctactcttgagtataccatttcaacaccaggcatcaccaaatcacacagaacaatagcaacgaagccttttagttccaagaaatttagggtagcctagagttgaaatctaacaaaacaaaagtcaaagctctatcacgtggatagttgttttccatgcactcttatttaagctaaatttttgggtatactacatccatttaattattgttttattgcttcttccctttgcctttcccccattactatcgcgtcttaagatcatactacgcactagtgtctttagaggtctctggtggacatgttcaaaccatctcaatcgaatcggtgttggacaagtttttcttgaatttgtgctacacctaacctatcacgtatatcatcgtttcaaacttgatccttcttgtatcatcataaatccaatgcaacatacgcatttctgcaacatttatctgttgaacatgtcatctttttgtaggttaacattatgcaccatacaatgtagcatgtctaatcatcatcctataaaatttacattttagcttatgtggtatcctcttgccacttagaacatcatatgtttgatgccatttcatccaccctgctttgattctatggctaacatcttcattaatatcctcgcctctctgtatcattggtcctaaatatggaaatacattctttctgggcactacttgaccttccaaactaacgtctcctttgatcctttcttgtgtgtagtagtaccgaagtcacatctcatatattcggttttagttctactaagtcccgggttcgatccccctcaggggtaaatttcgggcttggtaaaaaaaatcccctcgctgtgtcccgcccgctctcggggatcgatatcctgcgcgccaccctccggctgggcattgcagagtgggcagttgatcggctcgttagtgatggggagcggggttcaagggttttctcggccgggaccatgtttcggtctcttaatataataccgggagggcagtctttccctccccggtcgagttttagttctaccgagtctaaaacctttggactctagagtcccctgtcacaactcacaactctagttttctatttacttctacctagcgtttattaatgatcactatatcgtctgtaaaaagcatacaccaaggtaatccccttgtatgtcccttgtaatattatccatcacaagaaaaaaaaggtaaggctcaaagttgacttttgatataatcctattctaatcgagaagtcatctgtatcttcgtctcttgttcgaacactagtcacaattttttttgtacatgttcttaatgagtccaacgtaatattccttgatattttgtaagccctcatcaagtcaatgaaaatcacgtgtaggtccttcatttgttccttatactgctccatcacttgtctcattaagaaaatatctctcatagttaaccttttggcatgaaacaaaatcacacagaagttgtttcctttttttaagatcccacacaaaagaggtttgatctaaggaatctggatccctgacaggtttatcaaaatcctttgtgtttttcttaaaactgaatattcctccagcttctagtattgatgtaatattcaatctgtttagcaagtgaacaccttggttcttgttgttactgtacatcccacccacccccccgaggcccagattaccacgacatgaatacaagaatattgaacccagatctagagtttgtttgtactgttgaaaatcggtgacaattcattttgttattgcgctttctgataacgacaggactccgtgatgatgggagcggacacctatgaaactgaagaagaacgttcaaagctactgttagctgggaaggtcccagttggaataggaaggaacacaaagataaggtgagtatggatgtggaaccaccggttagttcccaaaaatatcactcactgatacctgatggtatcctctgattattttcaggaactgtatcattgacatgaatgctaggattgggaagaacgtggtgatcacaaacagtaaggtgagcgagcgcacctacatgggtgcagaatcttgtgtgctcatctatcctaattcggtaattcctatccagcgctagtcttgtgaccatggggcatgggttcgactctgtgacagggcatccaagaggctgatcacccggaagaagggtactacataaggtctggaatcgtggtgatcttgaagaatgcaaccatcaacgatgggtctgtcatatagatcggctgcgtttgcgtctacaaaacaagaacctacaatggtattgcatcgatggatcgtgtaaccttggtatggtaagagccgcttgacaggaagtcgagcgttcgggcgcaagatgcgttgtctggcatgctgttccttgaccatttgtgctgctagtatgtactgttataagctgccctagaagttgcagcaaacctttttatgaacctttgtatttccattacctgctttggatcaactatatctgtcatcctatatattactaaatttttacgtgtttttctaa

SEQ ID NO: 12 - protein, 672 aa, Zea mays
  MISC_FEATURE (1)..(672): modified sh2 protein
  MISC_FEATURE (249)..(249): T249K
  MISC_FEATURE (252)..(252): Q252G
  MISC_FEATURE (312)..(312): G312N
  MISC_FEATURE (317)..(317): D317G
  MISC_FEATURE (599)..(599): A599R
MGGQCCELEQCGNVGGSGDTLRERKEAGTSDYLAFCKRPNTQMLEKIYHVVKIRSAFPSANSSGRKVSKTMNGLQKAKAKGKIGTLMMVGCMSLSITCKKCEPLFLSSPPVCPLILYINTSTASQLATRSLREASAISISQPPFFCSVVYLVVGGDMQFALALDTNSGPHQIRSCEGDGIDRLEKLSIGGRKQEKALRNRCFGGRVAATTQCILTSDACPETLHSQTQSSRKNYADANRVSAIILGGGKGSGLFPLTSTRATPAVPVGGCYRLIDIPMSNCFNSGINKIFVMSQFNSTSLNRHIHRTYLEGNINFAGGSVQVLAATQMPEEPAGWFQGTADSIRKFIWVLEDYYSHKSIDNIVILSGDQLYRMNYMELVQKHVEDDADITISCAPVDESRASKNGLVKIDHTGRVLQFFEKPKGADLNSMRVETNFLSYAIDDAQKYPYLASMGIYVFKKDALLDLLKSKYIQLHDFGSEILPRAVLDHSVQACIFTGYWEDVGTIKSFFDANLALTEQPSKFDFYDPKTPFFTAPRCLPPTQLDKCKMKDAFISDGCLLRECNIEHSVIGVCSRVSSGCELKDSVMMGADTYETEEERSKLLLAGKVPVGIGRNTKIRNCIIDMNARIGKNVVITNSKGIQEADHPEEGYYIRSGIVVILKNATINDGSVI

SEQ ID NO: 13 - DNA, 9365 bp, Zea mays
  misc_feature (1)..(9365): modified sh2 genomic DNA
  misc_feature (4283)..(4285): T249K modification
  misc_feature (4292)..(4294): Q252G modification
  misc_feature (8654)..(8656): A599R modification
atgggcgggcagtgctgtgagttagagcagtgtggcaatgttggtggtagtggtgacacccttcgggagcggaaggaggctggaacttcaggtcagttctattgccataaataaataaaataaaataaatgaggctcaaaattccaaaatgtcatttcagcgtgtgcaccgcttcataaggccagtctcgtgaatcctacaagtttgatggtattctgggcctcgggcatagcccatttatcataaataaggacaattttgtaattctctccttttctttttgtttgcttgtatttgtatttcacatgggactcacttatgttgatgtattttagcttgtaaaaaagagtaaaaacattattcagtgaagtgacgcattgagttcatgattaagtttcaattatcctttttaataggtccatgccacgcttgtataaggattgagtaaaaaaactaactttattacaccacagtagataacaataagtcatatcagttgcatcacacaagcacactatttacttcttgtttttggatcaattctttggtgtctgtaaacccatacaacataacttgaatatgaacagcactgcagggataattgaaacctaggagtaaataaaatggatgtgaacgtttcagattatttggcattctgtaaaagacccaacacccaaatgcttgaaaaaatatatcatgttgtaaaaattcgatctgcatttccttctgtaagcaataaaacatggctcttttaagaattaccttgacaaaaaggtaaaatataatatcaataataactccggataggaaaaacttattcttctgcttataattatatcttaaatttaagtctatgacaatgtgaaatatttattgctagtgctacattttaatgtgtaatgaaagcactttcttttggacattcgaattctcctttgaaaatgatatactcctgtgattctggtttttaggagaattgcatgcatggcctagtggtagcgttgagataatgagttaagctttttgaagtttctaatgtgaattgtttttcttttaacttaaatactcattcagcgaaagatcctctagcgtaatgggtaaggattccgagtatcaccttcaggttctgggttcgaccctcatcgggggcgaatttcgggcttggttaaaaaaaatactctcactatgccccgcccgctcccgggttacgttctgtgcgccaccctctggctgagctgttgtaaagtgggcgatgacggcccgctagtgatggggggcaggggtggacccaagtacggctgagtgagggcacatgcccccgcccatttctttgttctaagtactagggtctaaattttcaccgtgagcccccctgcttagtctaatttagatgcccttgctctggtattttgatgctcgtccttacaagtatgcccttagtcatattttgtcctgggtccgcccttgatgagaggggccagggtttagagattttctcggccgggaccaatgttccggtctcttcttaatatagtaccgggacagtctttccctctccggccgagttttttatactcactctaggtgcaaacgtaagttgttcaggcttgtgcttaaggattaagaaagtaaataaaatgaccatgttacactttttttatttataccgtatcgggaaagataaaccatttatctgtgagtggtagcatggtcaaggagaaaaaaatagatgcatagaggttctgggacgactcacatttagagcatagttgaaaaggctaaaacaaccaggagggggtggggtcgggatacataaattttcatgagtgatggttgaactattgagggtgcaaaataattttttcaacaaataatgtggtgaaatacctaagaggggtgcacctagcatagattttttggggctccccttggcctctcctttcttccgccctgaaaacaacctacatggatacatctgcaaccagagggagtatctgatgctttttcctgggcagggagagctatgaggcgtatgtcctcaaagccactttgcattgtgtgaaaccaatatcgatctttgttacttcatcatgcgtgaacatttgtggaaactactagcttacaagcattagtgacagctcagaaaaaagttatctctgaaaggtttcatgtgtaccgtgggaaatgagaaatgttgccaactcaaacaccttcaatatgttgtttgcaggcaaactcttctggaagaaaggtgtctaaaactatgaacgggttacagaaaggtataaaccacggctgtgcattttggaagtatcatctatagatgtctgttgaggggaaagccgtacgccaacgttatttactcagaaacagcttcaacacacagttgtctgctttatgatggcatctccacccaggcacccaccatcacctatctctcgtgcctgtttattttcttgccctttctgatcataaaaaatcattaagagtttgcaaacatgcataggcatatcaatatgctcatttattaatttgctagcagatcatcttcctactctttactttatttattgtttgaaaaatatgtcctgcacctagggagctcgtatacagtaccaatgcatcttcattaaatgtgaatttcagaaaggaagtaggaacctatgagagtatttttcaaaattaattagcggcttctattatgtttatagcaaaggccaagggcaaaattggaacactaatgatggttggttgcatgagtctgtcgattacttgcaagaaatgtgaacctttgtttctgtgcgtgggcataaaacaaacagcttctagcctcttttacggtacttgcacttgcaagaaatgtgaactccttttcatttctgtatgtggacataatgccaaagcatccaggctttttcatggttgttgatgtctttacacagttcatctccaccagtatgccctctcatactctatataaacacatcaacagcatcgcaattagccacaagatcacttcgggaggcaagtgcgatttcgatctcgcagccaccttttttttgttctgttgtaagtatactttcccttaccatctttatctgttagtttaatttgtaattgggaagtattagtggaaagaggatgagatgctatcatctatgtactctgcaaatgcatctgacgttatatgggctgcttcatataatttgaattgctccattcttgccgacaatatattgcaaggtatatgcctagttccatcaaaagttctgttttttcattctaaaagcattttagtggcacgcattttttgtccatgagggaaaggaaatctgttttggttactttgcttgaggtgcattcttcatatgtccagttttatggaagtaataaacttcagtttggtcataagatgtcatattaaagggcaaacatatattcaatgttcaattcatcgtaaatgttccctttttgtaaaagattgcatactcatttatttgagttgcaggtgtatctagtagttggaggagatatgcagtttgcacttgcattggacacgaactcaggtcctcaccagataagatcttgtgagggtgatgggattgacaggttggaaaaattaagtattgggggcagaaagcaggagaaagctttgagaaataggtgctttggtggtagagttgctgcaactacacaatgtattcttacctcagatgcttgtcctgaaactcttgtaagtatccacctcaattattactcttacatgttggtttactttacgtttgtcttttcaagggaaatttactgtattttttgggttttgtgggagttctatacttctgttggactggttattgtaaagatttgttcaaatagggtcatctaataattgtttgaaatctgggaactgtggtttcactgcgttcaggaaaaagtgaattcttggttactgcatgaataacttatggaaatagaccttagagttgctgcatgattatcacaaatcattgctacgatatcttataatagttctttcgacctcgcattacatatataactgcaactcgtagttgcgttcaaaaaaaaatgcaactcttagaacgctcaccagtgtaatctttcctgaattgttatttaatggcatgtatgcactacttgtatacttatctaggattaagtaatctaactctaggccccatatttgcagcattctcaaacacagtcctctaggaaaaattatgctgatgcaaaccgtgtatctgctatcattttgggcggaggcaaaggatctgggctctttcctctgacaagcacaagagctacgcctgctgtaagggataacactgaacatccaacgttgattactctattatagtattatacagactgtacttttcgaatttatcttagttttctacaatatttagtggattcttctcattttcaagatacacaattgatccataatcgaagtggtatgtaagacagtgagttaaaagattatattttttgggagacttccagtcaaattttcttagaagtttttttggtccagatgttcataaagtcgccgctttcatactttttttaattttttaattggtgcactattaggtacctgttggaggatgttacaggcttattgatatccctatgagtaactgcttcaacagtggtataaataagatatttgtgatgagtcagttcaattctacttcgcttaaccgccatattcatcgtacataccttgaaggcgggatcaactttgctgatggatctgtacaggtgatttacctcatcttgttgatgtgtaatactgtaattaggagtagatttgtgtggagagaataataaacagatgccgagattcttctctaaaagtctagatccaaaggcattgtggttcaaaacactatggacttctaccatttatgtcattactttgccttaatgttccattgaatggggcaaattattgattctacaagtgtttaattaaaaactaattgttcatcctgcaggtattagcggctacacaaatgcctgaagagccagctggatggttccagggtacagcagactctatcagaaaatttatctgggtactcgaggtagttgatattttctcgtttatgaatgtccattcactcattcctgtagcattgtttctttgtaattttgagttctcctgtatttctttaggattattacagtcacaaatccattgacaacattgtaatcttgagtggcgatcagctttatcggatgaattacatggaacttgtgcaggtatggtgttctcttgttcctcatgtttcacgtaatgtcctgattttggattaaccaactacttttggcatgcattatttccagaaacatgtcgaggacgatgctgatatcactatatcatgtgctcctgttgatgagaggtaatcagttgtttatatcatcctaatatgaatatgtcatcttgttatccaacacaggatgcatatggtctaatctgctttcctttttttcccttcggaagccgagcttctaaaaatgggctagtgaagattgatcatactggacgtgtacttcaattctttgaaaaaccaaagggtgctgatttgaattctatggttagaaattccttgtgtaatccaattcttttgttttcctttctttcttgagatgaacccctcttttagttatttccatggataacctgtacttgacttattcagaaatgattttctattttgctgtagaatctgacactaaagctaatagctactgatgttgcagagagttgagaccaacttcctgagctatgctatagatgatgcacagaaatatccataccttgcatcaatgggcatttatgtcttcaagaaagatgcacttttagaccttctcaagtaatcactttcctgtgacttttttctatccaactcctagtttaccttctaacagtgtcaattcttaggtcaaaatatattcaattacatgactttggatctgaaatcctcccaagagctgtactagatcatagtgtgcaggtaagtctgatctgtctggagtatgtgttctgtaaactgtaaattcttcatgtcaaaaagttgtttttgtttccagtttccactagtttttatttaccaatgcacgatttatgtattttcgcttccatgcatcatacatactaacaatacattttacgtattgtgttaggcatgcatttttacgggctattgggaggatgttggaacaatcaaatcattctttgatgcaaacttggccctcactgagcaggtactctgtcatgtattctgtactgcatatatattacctggaattcaatgcatagaatgtgttagaccatcttagttccatcctgttttcttcaattagcttatcatttaatagttgttgactagaatctaaacacaaatttacctaatatgtttctctcttcagccttccaagtttgatttttatgatccaaaaacacctttcttcactgcaccccgatgcttgcctccgacgcaattggacaagtgcaaggtatatgtcttaccgagcacaattgttacctgagcaagattttgtgtacttgacttgttctcctccacagatgaaagatgcatttatctcagatggttgcttactgagagaatgcaacatcgagcattctgtgattggagtctgctcacgtgtcagctctggatgtgaactcaaggtacatactctgccaatgtatatgctgatgttttatacattctcttgcataatttgattcgagtcaccacaattagtgtaactgcaatctactcttgagtataccatttcaacaccaggcatcaccaaatcacacagaacaatagcaacgaagccttttagttccaagaaatttagggtagcctagagttgaaatctaacaaaacaaaagtcaaagctctatcacgtggatagttgttttccatgcactcttatttaagctaaatttttgggtatactacatccatttaattattgttttattgcttcttccctttgcctttcccccattactatcgcgtcttaagatcatactacgcactagtgtctttagaggtctctggtggacatgttcaaaccatctcaatcgaatcggtgttggacaagtttttcttgaatttgtgctacacctaacctatcacgtatatcatcgtttcaaacttgatccttcttgtatcatcataaatccaatgcaacatacgcatttctgcaacatttatctgttgaacatgtcatctttttgtaggttaacattatgcaccatacaatgtagcatgtctaatcatcatcctataaaatttacattttagcttatgtggtatcctcttgccacttagaacatcatatgtttgatgccatttcatccaccctgctttgattctatggctaacatcttcattaatatcctcgcctctctgtatcattggtcctaaatatggaaatacattctttctgggcactacttgaccttccaaactaacgtctcctttgatcctttcttgtgtgtagtagtaccgaagtcacatctcatatattcggttttagttctactaagtcccgggttcgatccccctcaggggtaaatttcgggcttggtaaaaaaaatcccctcgctgtgtcccgcccgctctcggggatcgatatcctgcgcgccaccctccggctgggcattgcagagtgggcagttgatcggctcgttagtgatggggagcggggttcaagggttttctcggccgggaccatgtttcggtctcttaatataataccgggagggcagtctttccctccccggtcgagttttagttctaccgagtctaaaacctttggactctagagtcccctgtcacaactcacaactctagttttctatttacttctacctagcgtttattaatgatcactatatcgtctgtaaaaagcatacaccaaggtaatccccttgtatgtcccttgtaatattatccatcacaagaaaaaaaaggtaaggctcaaagttgacttttgatataatcctattctaatcgagaagtcatctgtatcttcgtctcttgttcgaacactagtcacaattttttttgtacatgttcttaatgagtccaacgtaatattccttgatattttgtaagccctcatcaagtcaatgaaaatcacgtgtaggtccttcatttgttccttatactgctccatcacttgtctcattaagaaaatatctctcatagttaaccttttggcatgaaacaaaatcacacagaagttgtttcctttttttaagatcccacacaaaagaggtttgatctaaggaatctggatccctgacaggtttatcaaaatcctttgtgtttttcttaaaactgaatattcctccagcttctagtattgatgtaatattcaatctgtttagcaagtgaacaccttggttcttgttgttactgtacatcccacccacccccccgaggcccagattaccacgacatgaatacaagaatattgaacccagatctagagtttgtttgtactgttgaaaatcggtgacaattcattttgttattgcgctttctgataacgacaggactccgtgatgatgggagcggacacctatgaaactgaagaagaacgttcaaagctactgttagctgggaaggtcccagttggaataggaaggaacacaaagataaggtgagtatggatgtggaaccaccggttagttcccaaaaatatcactcactgatacctgatggtatcctctgattattttcaggaactgtatcattgacatgaatgctaggattgggaagaacgtggtgatcacaaacagtaaggtgagcgagcgcacctacatgggtgcagaatcttgtgtgctcatctatcctaattcggtaattcctatccagcgctagtcttgtgaccatggggcatgggttcgactctgtgacagggcatccaagaggctgatcacccggaagaagggtactacataaggtctggaatcgtggtgatcttgaagaatgcaaccatcaacgatgggtctgtcatatagatcggctgcgtttgcgtctacaaaacaagaacctacaatggtattgcatcgatggatcgtgtaaccttggtatggtaagagccgcttgacaggaagtcgagcgttcgggcgcaagatgcgttgtctggcatgctgttccttgaccatttgtgctgctagtatgtactgttataagctgccctagaagttgcagcaaacctttttatgaacctttgtatttccattacctgctttggatcaactatatctgtcatcctatatattactaaatttttacgtgtttttctaa

SEQ ID NO: 14 - protein, 672 aa, Zea mays
  MISC_FEATURE (1)..(672): modified sh2 protein
  MISC_FEATURE (249)..(249): T249K
  MISC_FEATURE (252)..(252): Q252G
  MISC_FEATURE (599)..(599): A599R
MGGQCCELEQCGNVGGSGDTLRERKEAGTSDYLAFCKRPNTQMLEKIYHVVKIRSAFPSANSSGRKVSKTMNGLQKAKAKGKIGTLMMVGCMSLSITCKKCEPLFLSSPPVCPLILYINTSTASQLATRSLREASAISISQPPFFCSVVYLVVGGDMQFALALDTNSGPHQIRSCEGDGIDRLEKLSIGGRKQEKALRNRCFGGRVAATTQCILTSDACPETLHSQTQSSRKNYADANRVSAIILGGGKGSGLFPLTSTRATPAVPVGGCYRLIDIPMSNCFNSGINKIFVMSQFNSTSLNRHIHRTYLEGGINFADGSVQVLAATQMPEEPAGWFQGTADSIRKFIWVLEDYYSHKSIDNIVILSGDQLYRMNYMELVQKHVEDDADITISCAPVDESRASKNGLVKIDHTGRVLQFFEKPKGADLNSMRVETNFLSYAIDDAQKYPYLASMGIYVFKKDALLDLLKSKYIQLHDFGSEILPRAVLDHSVQACIFTGYWEDVGTIKSFFDANLALTEQPSKFDFYDPKTPFFTAPRCLPPTQLDKCKMKDAFISDGCLLRECNIEHSVIGVCSRVSSGCELKDSVMMGADTYETEEERSKLLLAGKVPVGIGRNTKIRNCIIDMNARIGKNVVITNSKGIQEADHPEEGYYIRSGIVVILKNATINDGSVI

SEQ ID NO: 15 - DNA, 9365 bp, Zea mays
  misc_feature (1)..(9365): modified sh2 genomic DNA
  misc_feature (4756)..(4758): G312N modification
  misc_feature (4771)..(4773): D317G modification
  misc_feature (8654)..(8656): A599R modification
atgggcgggcagtgctgtgagttagagcagtgtggcaatgttggtggtagtggtgacacccttcgggagcggaaggaggctggaacttcaggtcagttctattgccataaataaataaaataaaataaatgaggctcaaaattccaaaatgtcatttcagcgtgtgcaccgcttcataaggccagtctcgtgaatcctacaagtttgatggtattctgggcctcgggcatagcccatttatcataaataaggacaattttgtaattctctccttttctttttgtttgcttgtatttgtatttcacatgggactcacttatgttgatgtattttagcttgtaaaaaagagtaaaaacattattcagtgaagtgacgcattgagttcatgattaagtttcaattatcctttttaataggtccatgccacgcttgtataaggattgagtaaaaaaactaactttattacaccacagtagataacaataagtcatatcagttgcatcacacaagcacactatttacttcttgtttttggatcaattctttggtgtctgtaaacccatacaacataacttgaatatgaacagcactgcagggataattgaaacctaggagtaaataaaatggatgtgaacgtttcagattatttggcattctgtaaaagacccaacacccaaatgcttgaaaaaatatatcatgttgtaaaaattcgatctgcatttccttctgtaagcaataaaacatggctcttttaagaattaccttgacaaaaaggtaaaatataatatcaataataactccggataggaaaaacttattcttctgcttataattatatcttaaatttaagtctatgacaatgtgaaatatttattgctagtgctacattttaatgtgtaatgaaagcactttcttttggacattcgaattctcctttgaaaatgatatactcctgtgattctggtttttaggagaattgcatgcatggcctagtggtagcgttgagataatgagttaagctttttgaagtttctaatgtgaattgtttttcttttaacttaaatactcattcagcgaaagatcctctagcgtaatgggtaaggattccgagtatcaccttcaggttctgggttcgaccctcatcgggggcgaatttcgggcttggttaaaaaaaatactctcactatgccccgcccgctcccgggttacgttctgtgcgccaccctctggctgagctgttgtaaagtgggcgatgacggcccgctagtgatggggggcaggggtggacccaagtacggctgagtgagggcacatgcccccgcccatttctttgttctaagtactagggtctaaattttcaccgtgagcccccctgcttagtctaatttagatgcccttgctctggtattttgatgctcgtccttacaagtatgcccttagtcatattttgtcctgggtccgcccttgatgagaggggccagggtttagagattttctcggccgggaccaatgttccggtctcttcttaatatagtaccgggacagtctttccctctccggccgagttttttatactcactctaggtgcaaacgtaagttgttcaggcttgtgcttaaggattaagaaagtaaataaaatgaccatgttacactttttttatttataccgtatcgggaaagataaaccatttatctgtgagtggtagcatggtcaaggagaaaaaaatagatgcatagaggttctgggacgactcacatttagagcatagttgaaaaggctaaaacaaccaggagggggtggggtcgggatacataaattttcatgagtgatggttgaactattgagggtgcaaaataattttttcaacaaataatgtggtgaaatacctaagaggggtgcacctagcatagattttttggggctccccttggcctctcctttcttccgccctgaaaacaacctacatggatacatctgcaaccagagggagtatctgatgctttttcctgggcagggagagctatgaggcgtatgtcctcaaagccactttgcattgtgtgaaaccaatatcgatctttgttacttcatcatgcgtgaacatttgtggaaactactagcttacaagcattagtgacagctcagaaaaaagttatctctgaaaggtttcatgtgtaccgtgggaaatgagaaatgttgccaactcaaacaccttcaatatgttgtttgcaggcaaactcttctggaagaaaggtgtctaaaactatgaacgggttacagaaaggtataaaccacggctgtgcattttggaagtatcatctatagatgtctgttgaggggaaagccgtacgccaacgttatttactcagaaacagcttcaacacacagttgtctgctttatgatggcatctccacccaggcacccaccatcacctatctctcgtgcctgtttattttcttgccctttctgatcataaaaaatcattaagagtttgcaaacatgcataggcatatcaatatgctcatttattaatttgctagcagatcatcttcctactctttactttatttattgtttgaaaaatatgtcctgcacctagggagctcgtatacagtaccaatgcatcttcattaaatgtgaatttcagaaaggaagtaggaacctatgagagtatttttcaaaattaattagcggcttctattatgtttatagcaaaggccaagggcaaaattggaacactaatgatggttggttgcatgagtctgtcgattacttgcaagaaatgtgaacctttgtttctgtgcgtgggcataaaacaaacagcttctagcctcttttacggtacttgcacttgcaagaaatgtgaactccttttcatttctgtatgtggacataatgccaaagcatccaggctttttcatggttgttgatgtctttacacagttcatctccaccagtatgccctctcatactctatataaacacatcaacagcatcgcaattagccacaagatcacttcgggaggcaagtgcgatttcgatctcgcagccaccttttttttgttctgttgtaagtatactttcccttaccatctttatctgttagtttaatttgtaattgggaagtattagtggaaagaggatgagatgctatcatctatgtactctgcaaatgcatctgacgttatatgggctgcttcatataatttgaattgctccattcttgccgacaatatattgcaaggtatatgcctagttccatcaaaagttctgttttttcattctaaaagcattttagtggcacgcattttttgtccatgagggaaaggaaatctgttttggttactttgcttgaggtgcattcttcatatgtccagttttatggaagtaataaacttcagtttggtcataagatgtcatattaaagggcaaacatatattcaatgttcaattcatcgtaaatgttccctttttgtaaaagattgcatactcatttatttgagttgcaggtgtatctagtagttggaggagatatgcagtttgcacttgcattggacacgaactcaggtcctcaccagataagatcttgtgagggtgatgggattgacaggttggaaaaattaagtattgggggcagaaagcaggagaaagctttgagaaataggtgctttggtggtagagttgctgcaactacacaatgtattcttacctcagatgcttgtcctgaaactcttgtaagtatccacctcaattattactcttacatgttggtttactttacgtttgtcttttcaagggaaatttactgtattttttgggttttgtgggagttctatacttctgttggactggttattgtaaagatttgttcaaatagggtcatctaataattgtttgaaatctgggaactgtggtttcactgcgttcaggaaaaagtgaattcttggttactgcatgaataacttatggaaatagaccttagagttgctgcatgattatcacaaatcattgctacgatatcttataatagttctttcgacctcgcattacatatataactgcaactcgtagttgcgttcaaaaaaaaatgcaactcttagaacgctcaccagtgtaatctttcctgaattgttatttaatggcatgtatgcactacttgtatacttatctaggattaagtaatctaactctaggccccatatttgcagcattctcaaacacagtcctctaggaaaaattatgctgatgcaaaccgtgtatctgctatcattttgggcggaggcactggatctcagctctttcctctgacaagcacaagagctacgcctgctgtaagggataacactgaacatccaacgttgattactctattatagtattatacagactgtacttttcgaatttatcttagttttctacaatatttagtggattcttctcattttcaagatacacaattgatccataatcgaagtggtatgtaagacagtgagttaaaagattatattttttgggagacttccagtcaaattttcttagaagtttttttggtccagatgttcataaagtcgccgctttcatactttttttaattttttaattggtgcactattaggtacctgttggaggatgttacaggcttattgatatccctatgagtaactgcttcaacagtggtataaataagatatttgtgatgagtcagttcaattctacttcgcttaaccgccatattcatcgtacataccttgaaggcaacatcaactttgctggtggatctgtacaggtgatttacctcatcttgttgatgtgtaatactgtaattaggagtagatttgtgtggagagaataataaacagatgccgagattcttctctaaaagtctagatccaaaggcattgtggttcaaaacactatggacttctaccatttatgtcattactttgccttaatgttccattgaatggggcaaattattgattctacaagtgtttaattaaaaactaattgttcatcctgcaggtattagcggctacacaaatgcctgaagagccagctggatggttccagggtacagcagactctatcagaaaatttatctgggtactcgaggtagttgatattttctcgtttatgaatgtccattcactcattcctgtagcattgtttctttgtaattttgagttctcctgtatttctttaggattattacagtcacaaatccattgacaacattgtaatcttgagtggcgatcagctttatcggatgaattacatggaacttgtgcaggtatggtgttctcttgttcctcatgtttcacgtaatgtcctgattttggattaaccaactacttttggcatgcattatttccagaaacatgtcgaggacgatgctgatatcactatatcatgtgctcctgttgatgagaggtaatcagttgtttatatcatcctaatatgaatatgtcatcttgttatccaacacaggatgcatatggtctaatctgctttcctttttttcccttcggaagccgagcttctaaaaatgggctagtgaagattgatcatactggacgtgtacttcaattctttgaaaaaccaaagggtgctgatttgaattctatggttagaaattccttgtgtaatccaattcttttgttttcctttctttcttgagatgaacccctcttttagttatttccatggataacctgtacttgacttattcagaaatgattttctattttgctgtagaatctgacactaaagctaatagctactgatgttgcagagagttgagaccaacttcctgagctatgctatagatgatgcacagaaatatccataccttgcatcaatgggcatttatgtcttcaagaaagatgcacttttagaccttctcaagtaatcactttcctgtgacttttttctatccaactcctagtttaccttctaacagtgtcaattcttaggtcaaaatatattcaattacatgactttggatctgaaatcctcccaagagctgtactagatcatagtgtgcaggtaagtctgatctgtctggagtatgtgttctgtaaactgtaaattcttcatgtcaaaaagttgtttttgtttccagtttccactagtttttatttaccaatgcacgatttatgtattttcgcttccatgcatcatacatactaacaatacattttacgtattgtgttaggcatgcatttttacgggctattgggaggatgttggaacaatcaaatcattctttgatgcaaacttggccctcactgagcaggtactctgtcatgtattctgtactgcatatatattacctggaattcaatgcatagaatgtgttagaccatcttagttccatcctgttttcttcaattagcttatcatttaatagttgttgactagaatctaaacacaaatttacctaatatgtttctctcttcagccttccaagtttgatttttatgatccaaaaacacctttcttcactgcaccccgatgcttgcctccgacgcaattggacaagtgcaaggtatatgtcttaccgagcacaattgttacctgagcaagattttgtgtacttgacttgttctcctccacagatgaaagatgcatttatctcagatggttgcttactgagagaatgcaacatcgagcattctgtgattggagtctgctcacgtgtcagctctggatgtgaactcaaggtacatactctgccaatgtatatgctgatgttttatacattctcttgcataatttgattcgagtcaccacaattagtgtaactgcaatctactcttgagtataccatttcaacaccaggcatcaccaaatcacacagaacaatagcaacgaagccttttagttccaagaaatttagggtagcctagagttgaaatctaacaaaacaaaagtcaaagctctatcacgtggatagttgttttccatgcactcttatttaagctaaatttttgggtatactacatccatttaattattgttttattgcttcttccctttgcctttcccccattactatcgcgtcttaagatcatactacgcactagtgtctttagaggtctctggtggacatgttcaaaccatctcaatcgaatcggtgttggacaagtttttcttgaatttgtgctacacctaacctatcacgtatatcatcgtttcaaacttgatccttcttgtatcatcataaatccaatgcaacatacgcatttctgcaacatttatctgttgaacatgtcatctttttgtaggttaacattatgcaccatacaatgtagcatgtctaatcatcatcctataaaatttacattttagcttatgtggtatcctcttgccacttagaacatcatatgtttgatgccatttcatccaccctgctttgattctatggctaacatcttcattaatatcctcgcctctctgtatcattggtcctaaatatggaaatacattctttctgggcactacttgaccttccaaactaacgtctcctttgatcctttcttgtgtgtagtagtaccgaagtcacatctcatatattcggttttagttctactaagtcccgggttcgatccccctcaggggtaaatttcgggcttggtaaaaaaaatcccctcgctgtgtcccgcccgctctcggggatcgatatcctgcgcgccaccctccggctgggcattgcagagtgggcagttgatcggctcgttagtgatggggagcggggttcaagggttttctcggccgggaccatgtttcggtctcttaatataataccgggagggcagtctttccctccccggtcgagttttagttctaccgagtctaaaacctttggactctagagtcccctgtcacaactcacaactctagttttctatttacttctacctagcgtttattaatgatcactatatcgtctgtaaaaagcatacaccaaggtaatccccttgtatgtcccttgtaatattatccatcacaagaaaaaaaaggtaaggctcaaagttgacttttgatataatcctattctaatcgagaagtcatctgtatcttcgtctcttgttcgaacactagtcacaattttttttgtacatgttcttaatgagtccaacgtaatattccttgatattttgtaagccctcatcaagtcaatgaaaatcacgtgtaggtccttcatttgttccttatactgctccatcacttgtctcattaagaaaatatctctcatagttaaccttttggcatgaaacaaaatcacacagaagttgtttcctttttttaagatcccacacaaaagaggtttgatctaaggaatctggatccctgacaggtttatcaaaatcctttgtgtttttcttaaaactgaatattcctccagcttctagtattgatgtaatattcaatctgtttagcaagtgaacaccttggttcttgttgttactgtacatcccacccacccccccgaggcccagattaccacgacatgaatacaagaatattgaacccagatctagagtttgtttgtactgttgaaaatcggtgacaattcattttgttattgcgctttctgataacgacaggactccgtgatgatgggagcggacacctatgaaactgaagaagaacgttcaaagctactgttagctgggaaggtcccagttggaataggaaggaacacaaagataaggtgagtatggatgtggaaccaccggttagttcccaaaaatatcactcactgatacctgatggtatcctctgattattttcaggaactgtatcattgacatgaatgctaggattgggaagaacgtggtgatcacaaacagtaaggtgagcgagcgcacctacatgggtgcagaatcttgtgtgctcatctatcctaattcggtaattcctatccagcgctagtcttgtgaccatggggcatgggttcgactctgtgacagggcatccaagaggctgatcacccggaagaagggtactacataaggtctggaatcgtggtgatcttgaagaatgcaaccatcaacgatgggtctgtcatatagatcggctgcgtttgcgtctacaaaacaagaacctacaatggtattgcatcgatggatcgtgtaaccttggtatggtaagagccgcttgacaggaagtcgagcgttcgggcgcaagatgcgttgtctggcatgctgttccttgaccatttgtgctgctagtatgtactgttataagctgccctagaagttgcagcaaacctttttatgaacctttgtatttccattacctgctttggatcaactatatctgtcatcctatatattactaaatttttacgtgtttttctaa

SEQ ID NO: 16 - protein, 672 aa, Zea mays
  MISC_FEATURE (1)..(672): modified sh2 protein
  MISC_FEATURE (312)..(312): G312N
  MISC_FEATURE (317)..(317): D317G
  MISC_FEATURE (599)..(599): A599R
MGGQCCELEQCGNVGGSGDTLRERKEAGTSDYLAFCKRPNTQMLEKIYHVVKIRSAFPSANSSGRKVSKTMNGLQKAKAKGKIGTLMMVGCMSLSITCKKCEPLFLSSPPVCPLILYINTSTASQLATRSLREASAISISQPPFFCSVVYLVVGGDMQFALALDTNSGPHQIRSCEGDGIDRLEKLSIGGRKQEKALRNRCFGGRVAATTQCILTSDACPETLHSQTQSSRKNYADANRVSAIILGGGTGSQLFPLTSTRATPAVPVGGCYRLIDIPMSNCFNSGINKIFVMSQFNSTSLNRHIHRTYLEGNINFAGGSVQVLAATQMPEEPAGWFQGTADSIRKFIWVLEDYYSHKSIDNIVILSGDQLYRMNYMELVQKHVEDDADITISCAPVDESRASKNGLVKIDHTGRVLQFFEKPKGADLNSMRVETNFLSYAIDDAQKYPYLASMGIYVFKKDALLDLLKSKYIQLHDFGSEILPRAVLDHSVQACIFTGYWEDVGTIKSFFDANLALTEQPSKFDFYDPKTPFFTAPRCLPPTQLDKCKMKDAFISDGCLLRECNIEHSVIGVCSRVSSGCELKDSVMMGADTYETEEERSKLLLAGKVPVGIGRNTKIRNCIIDMNARIGKNVVITNSKGIQEADHPEEGYYIRSGIVVILKNATINDGSVI

SEQ ID NO: 17 - protein, 516 aa, Saccharum hybrid cultivar R570
  MISC_FEATURE (1)..(516): AGPase large subunit
MQFALALDANSGPHPIRSCEGGGIDRSERLSIGGSKQEKALRNRCFGGRVAATTQCILTSDACPETLHFQTQSSRKSYADANRVSAIILGEGTGSQLFPLTSTRATPAVPVGGCYRLIDIPMSNCFNSGINKIFVMSQFNSTSLNRHIHRTYLGGGINFADGSVQVLAATQMPEEPAGWFQGTADSIRKFIWILEDYYNHKSIEHIVILSGDQLHRMNYMELVQKHVEDNADITISCAPVDESRASNNGLVKFDHTGRVLQFFEKPKGADLNSMRVDTNFLSYAIADAQKYPYIASMGIYVFKKDALLDLLKSKYAQLHDFESEILPRAVVDHSVQACIFMGYWEDAGTIKSFFDANLALTEQLSKFDFYDPKTPFFTAPRYLPPTELDKCKIKDASISDGCLLRECSIEHSVIGVCSRVSYGCELKDCVMMGADIYETEEEASKLLLAGKVPVGIGRNTKIRNCIIDMNARIGKNVVITNSKGIQEADHPEEGYYIRSGIVVILKNATIKDGSVI

SEQ ID NO: 18 - protein, 517 aa, Sorghum bicolor
  MISC_FEATURE (1)..(517): AGPase large subunit
MQFSLASDANSGPHPIRRSCEGGGIDRLERLSIGGSKQEKALRNRCFGGRVAATTQCILTSDACPETLHFQTQSSRKSYADANHVSAIILGGGTGSQLFPLTSTRATPAVPVGGCYRLIDIPMSNCFNSGINKIFVMTQFNSTSLNRHIHRTYLGGEINFADGSVQVLADTQMPEEPDGWFQGTADSVRKFIWVLEDYYNHKSIEHIVILSGDQLYQMNYMELVQKHVEDNADITVSCAPVDESRASNNGLVKCDHTGRVLQFFEKPKGADLNSMRVDTNFLSYAIGDAQKYQYIASMGIYVFKKDALLDLLKSKYTQLHDFGSEILPRAVLEHNVQTCIFMGYWEDVGTIKSFFDANLALTEQPSKFDFYDPKTPFFTAPRYLPPTQLDKCKIKDASISDGCLLRECSIEHSVIGVCSRVSYGCELKDCVMMGADIYETEEEASKLLLAGEVPVGIGGNTKIRNCIIDINARIGKNVVITNSKGIQEADHPEEGYYIKSGIVVILKNATIKDGSVI

SEQ ID NO: 19 - protein, 517 aa, Panicum hallii
  MISC_FEATURE (1)..(517): AGPase large subunit
MQFTLALDGNTSPHLIRRSSEGGGSERSMERLNIGVINQEKALRNRCFNGGAARTQCVLTSDSYPETLHFQTHSSRKSYADANRVSAVILGGGTGAQLFPLTSTRATPAVPLGGCHRLIDIPMSNCFNSGINKIFVMTQFNSASLNRHIHRTYLGGGINFTDGSVQVLAATQMPEEPAGWFQGTADAIRKFVWVLEDYYYHKDIEHIVILSGDQLYRMNYMELVQKHVDDNADITISCAPIDESRASNYGLVKFDYTGRVLQFFEKPKGADLESMRVDTNFLSYAIADTQKYPYIASMGVYVFKRDALLNLLKSKYSQLHDFGSEILPRAVLEHNVQAYIFTGYWEDVGTIKSFFDANLALTEQPSKFEFYDPKTPFFTAPRNLPPTQLDKCKIKDAFISDGCLLSECSIKHSVIGVCSRVSSRCELMDTMMMGADIYETEEETAKLLLAGKVPIGIGENTKIRNCIIDMNARIGKNVVIANSKGIQEADHPEEGYYIRFGIVLILKNATIKDGSVI

SEQ ID NO: 20 - protein, 518 aa, Setaria italica
  MISC_FEATURE (1)..(518): AGPase large subunit
MQFTLALDGNTSPHLMRRSGEGGGSEMLMERLNIGVINQEKALRNRCFSGGGARTTQCVLNSGACSETLHFQTHSSRKSYADANRVSAVILGGGIGAQLFPLTSTRATPAVPVGGCHRLIDIPMSNCFNSGINKIFVMTQFNSASLNRHIHRTYLGGGITFTDGSVQVLAATQMPEEPAGWFQGTADAIRKFVWVLEDYYNHKDIEHIVILSGDQLYRMNYMELVQKHVDDNAEITISCAPVNESRASNYGLVKFDYTGRVLQFFEKPVGADLESMRVDTNFLSYAIGDTQKYPYIASMGVYVFKRDALLDLLKSKYNQLHDFGSEILPRAVLEHNVQAYIFTGYWEDVGTIKSFFDANLALTEQPSKFEFYDPQTPFFTAPRYLPPTQLDKCKIKDAFISDGCLLSECTIKHSVIGVCSRLRSGCELMDTMMMGADIYETEEETSKLHLAGKVPIGIGENTKIRNCIIDMNARIGKNVVIANSKGIQEADHPEEGYYIRSGIVVILKNAIIKDGSVI

SEQ ID NO: 21 - protein, 409 aa, Zea diploperennis
  MISC_FEATURE (1)..(409): AGPase large subunit
MQFALALDTNSGPHQIRSCEGDGIDRLEQLSIGGRKQEKALRNRCFGGRVAATTQCILTSDACPETLHSQTQSSRKNYADANRVSAIILGGGTGSQLFPLTSTRATPAVPVGGCYRLIDIPMSNCFNSGINKIFVMSQFNSTSLNRHIHRTYLEGGINFADGSVQVLAATQMPEEPAGWFQGTADSIRKFIWVLEDYYSHKSIDNIVILSGDQLYRMNYMELVQKHVEDDADITISCAPVDESRASKNGLVKIDHTGRVLQFFEKPKGADLNSMRVETNFLSYAIDDAQKYPYLASMGIYVFKKDALLDLLKSKYTQLHDFGSEILPRAVVDHSVQACIFTGYWEDVGTIKSFFDANLALTEQPSKFDFYDPKTPFFTAPRCLPPTQLDKCKMKDAFISDGCLLRECNI

SEQ ID NO: 22 - protein, 518 aa, Oryza brachyantha
  MISC_FEATURE (1)..(518): AGPase large subunit
MQFMMPLDTNACAQPTRRAGESADTERLMERLNIGGMTQEKALRKRCFGDGVAGTARCVFTSDADGDTLHLQTQSSQKNYADASRVSAVILGGGTGVQLFPLTSTMATPAVPVGGCYRLIDIPMSNCFNSGINKIFVMTQFNSASLNRHIHRTYLGGGINFTDGSVQVLAATQMPEEPAGWFEGTADAIRKFMWILEDYYNQNNIEHIIILCGDQLYRMNYMELVQKHVDDNADITISCAPVDRSRASEYGLVKFDNSGRVIQFFEKPQGVDLESMKVDTSFLSYTIDDTQKYPYIASMGIYVLKKDVLLDILKSKYAHLRDFGSEILPRAVLEHNVKACVFTEYWEDIGTIKSFFKANLALTEQPPKFEFYDPKTPFFTSPRHLPPTKLEKCKIKDAIISDGCSFSECTIEHSVIGICSRLSIGCELKDTMMMGADLYETEEENSKLLLEGRVPIGVGENTKIRNCIIDMNARIGRNVVIANAEGVQEADHPEEGYYIRSGIVVILKNATIQDGTVI

SEQ ID NO: 23 - protein, 518 aa, Oryza sativa
  MISC_FEATURE (1)..(518): AGPase large subunit
MQFMMPLDTNACAQPMRRAGEGAGTERLMERLNIGGMTQEKALRKRCFGDGVTGTARCVFTSDADRDTPHLRTQSSRKNYADASRVSAVILGGGTGVQLFPLTSTRAAPAVPVGGCYRLIDIPMSNCFNSGINKIFVMTQFNSASLNRHIHHTYLGGGINFTDGSVQVLAATQMPDEPAGWFQGTADAIRKFMWILEDHYNQNNIEHVVILCGDQLYRMNYMELVQKHVDDNADITISCAPIDGSRASDYGLVKFDDSGRVIQFLEKPEGADLESMKVDTSFLSYAIDDKQKYPYIASMGIYVLKKDVLLDILKSKYAHLQDFGSEILPRAVLEHNVKACVFTEYWEDIGTIKSFFDANLALTEQPPKFEFYDPKTPFFTSPRYLPPARLEKCKIKDAIISDGCSFSECTIEHSVIGISSRVSIGCELKDTMMMGADQYETEEETSKLLFEGKVPIGIGENTKIRNCIIDMNARIGRNVIIANTQGVQESDHPEEGYYIRSGIVVILKNATIKDGTVI

SEQ ID NO: 24 - protein, 518 aa, Oryza sativa
  MISC_FEATURE (1)..(518): AGPase large subunit
MQFMMPLDTNACAQPMRRAGEGAGTERLMERLNIGGMTQEKALRKRCFGDGVTGTARCVFTSDADRDTPHLRTQSSRKNYADASHVSAVILGGGTGVQLFPLTSTRATPAVPVGGCYRLIDIPMSNCFNSGINKIFVMTQFNSASLNRHIHHTYLGGGINFTDGSVQVLAATQMPDEPAGWFQGTADAIRKFMWILEDHYNQNNIEHVVILCGDQLYRMNYMELVQKHVDDNADITISCAPIDGSRASDYGLVKFDDSGRVIQFLEKPEGADLESMKVDTSFLSYAIDDKQKYPYIASMGIYVLKKDVLLDILKSKYAHLQDFGSEILPRAVLEHNVKACVFTEYWEDIGTIKSFFDANLALTEQPPKFEFYDPKTPFFTSPRYLPPARLEKCKIKDAIISDGCSFSECTIEHSVIGISSRVSIGCELKDTMMMGADQYETEEETSKLLFEGKVPIGIGENTKIRNCIIDMNARIGRNVIIANTQGVQESDHPEEGYYIRSGIVVILKNATIKDGTVI

SEQ ID NO: 25 - protein, 519 aa, Eleusine coracana
  MISC_FEATURE (1)..(519): AGPase large subunit
MQFSTVFPLEGKACMSPVRRSSDGSRSDRVRIGDSSSLKHDRALRRMCFGARGPANSAQCVLTSDAGPDTLVVRTSFRRNYADPNEVAAVILGGGTGTQLFPLTSTRATPAVPIGGCYRLIDIPMSNCFNSGINKIFVMTQFNSASLNRHIHRTYLGGGINFTDGSVEVLAATQMPGEAAGWFQGTADAVRKFIWVLEDYYKHKAIEHILILSGDQLYRMDYMELVQKHVDDNADITLSCAPVGESRASDYGLVKFDSSGRVIQFSEKPKGADLEAMKVDTSFLNFAIDDTTKYPYIASMGVYVFKRDVLLNLLKSRYSELHDFGSEILPRALHEHNVQAYVFTDYWEDIGTIRSFFDANMALCEQPPKFQFYDPKTPFFTSPRYLPPTKSDKCKIKDSIISHGCFLRECTIEHSIVGVRSRLNSGCELKNTMMMGADMYETEDEIARLMSDGKVPIGVGENTKISNCIIDMNARVGRNITITNTEGVQEADRPEEGYYIRSGIVVILKNATIKDGTVI

SEQ ID NO: 26 - protein, 522 aa, Brachypodium distachyon
  MISC_FEATURE (1)..(522): AGPase large subunit
MSRMQFSSVLPLEGKACVSPLRRNVESSGSERIRIGDSSTIKHERALRRMCFGARGAASGAQCVLTSDASPDTLVVRTSFRRNYADPNEVAAVILGGGTGTQLFPLTSTRATPAVPIGGCYRLIDIPMSNCFNSGINKIFVMTQFNSASLNRHIHRTYLGGGINFTDGSVEVLAATQMPGEAAGWFRGTADAVRKFIWVLEDYYKHKSIEHILILSGDQLYRMDYMELVQKHVDDNADITLSCAPVGESRASEYGLVKFDSSGRVIQFSEKPKGVDLEAMKVDTSFLNFAIDDPAKFPYIASMGVYVFKRDVLLNLLKSRYAELHDFGSEILPRALHEHNVQAYVFTDYWEDIGTIRSFFDANMALCEQPPKFEFYDPKTPFFTSPRYLPPTKSDKCRIKEAIISHGCFLRECTIEHSIVGVRSRLNSGCELKNAMMMGADLYETEDEISRLLSEGKVPIGVGENAKISNCIIDMNARIGRDVIIANSEGVEEADRAEEGYYIRSGIVVILKNATIKDGTVV

SEQ ID NO: 27 - protein, 569 aa, Dichanthelium oligosanthes
  MISC_FEATURE (1)..(569): AGPase large subunit
MDEAFSPAAGPSGVWALAYHATSGQAVGSPRDSPAATRARLVAQSRCASAMQFSSVFPLEGKACVSPVRRGGEGSGSERVRIGDSSSVRQNRALRRICFGARGTANGAQCVLTSDAGPDTLVVRTSFRRHYADPNEVAAVILGGGTGTQLFPLTSTRATPAVPIGGCYRLIDIPMSNCFNSGINKIFVMTQFNSASLNRHIHRTYLGGGINFTDGSVEVLAATQMPGEAPGWFQGTADAVRKFIWVLEDYYKHKAIEHILILSGDQLYRMDYMELVQKHVDDNADITLSCAPVGESRASDYGLVKFDSSGRVIQFSEKPKGSDLEEMKVDTSFLNFAIDDPTKYPYIASMGVYVFKRDVLLNLLKLRYAELHDFGSEILPKALHEHNVQAYVFTDYWEDIGTIRSFFDANMALCEQPPKFEFYDPKTPFFTSPRYLPPTKSDKCRIKDAIISHGCFLRECTIEHSIVGVRSRLNSGCELKNTMMMGADLYETEDEISRLLSEGKVPIGVGENTKISNCIIDMNARVGRNVSITNSEGVQEADWPEEGYYIRSGIVVILKNSTIKDGTVI

SEQ ID NO: 28 - protein, 522 aa, Triticum monococcum
  MISC_FEATURE (1)..(522): AGPase large subunit
MSSMQFSSVLPLEGKACVSPVRREGSACERLKVGDSSSIRHERASRRMCNGGRGPAATGAQCVLTSDASPADTLVLRTSFRRNYADPNEVAAVILGGGTGTQLFPLTSTRATPAVPIGGCYRLIDIPMSNCFNSGINKIFVMTQFNSASLNRHIHRTYLGGGINFTDGSVEVLAATQMPGEAAGWFRGTADAVRKFIWVLEDYYKNKSIEHILILSGDQLYRMDYMELVQKHVDDNADITLSCAPVGESRASEYGLVKFDSSGRVVQFSEKPKGADLEAMKVDTSFLNFAIDDPAKYPYIASMGVYVFKRDVLLNLLKSRYAELHDFGSEILPRALHDHNVQAYVFTDYWEDIGTIRSFFDANMALCEQPPKFEFYDPKTPFFTSPRYLPPTKSDKCRIKEAIISHGCFLRECKIEHSIIGVRSRLNSGSELKNAMMMGADSYETEDEISRLMSEGKVPIGVGENTKISNCIIDMNARIGRDVVISNKEGVQEADRPEEGYYIRSGIVVIQKNATIKDGTVV

SEQ ID NO: 29 - protein, 522 aa, Aegilops bicornis
  MISC_FEATURE (1)..(522): AGPase large subunit
MSSMQFSSVLPLEGKACVSPVRREGSASERLKVGDSSSIRHERASRRMCNGGRGPAATGAQCVLTSDASPADTLVLRTSFRRNYADPNEVAAVILGGGTGTQLFPLTSTRATPAVPIGGCYRLIDIPMSNCFNSGINKIFVMTQFNSASLNRHIHRTYLGGGINFTDGSVEVLAATQMPGEAAGWFRGTADAVRKFIWVLEDYYKNKSIEHILILSGDQLYRMDYMELVQKHVDDNADITLSCAPVGESRASEYGLVKFDSSGRVVQFSEKPKGADLEAMKVDTSFLNFAIDDTAKYPYIASMGVYVFKRDVLLNLLKSRYAELHDFGSEILPRALHDHNVQAYVFTDYWEDIGTIRSFFDANMALCEQPPKFEFYDPKTPFFTSPRYLPPTKSDKCRIKEAIISHGCFLRECKIEHSIIGVRSRLNSGSELKNAMMMGADSYETEDEISRLMSEGKVPIGVGENTKISNCIIDMNARIGRDVVISNKEGVQEADRPEEGYYIRSGIVVIQKNATIKDGTNL

SEQ ID NO: 30 - protein, 522 aa, Aegilops tauschii
  MISC_FEATURE (1)..(522): AGPase large subunit
MSSMQFSSVLPLEGKACVSPVRREGSASERLKVGDSSSIRHERASRRMCNGGRGPAATGAQCVLTSDASPADTLVLRTSFRRNYADPNEVAAVILGGGTGTQLFPLTSTRATPAVPIGGCYRLIDIPMSNCFNSGINKIFVMTQFNSASLNRHIHRTYLGGGINFTDGSVEVLAATQMPGEAAGWFRGTADAVRKFIWVLEDYYKNKSIEHILILSGDQLYRMDYMELVQKHVDDNADITLSCAPVGESRASEYGLVKFDSSGRVVQFSEKPKGDDLEAMKVDTSFLNFAIDDPAKYPYIASMGVYVFKRDVLLNLLKSRYAELHDFGSEILPRALHDHNVQAYVFTDYWEDIGTIRSFFDANMALCEQPPKFEFYDPKTPFFTSPRYLPPTKSDKCRIKEAIISHGCFLRECKIEHSIIGVRSRLNSGSELKNAMMMGADSYETEDEISRLMSEGKVPIGVGENTKISNCIIDMNARIGRDVVISNKEGVQEADRPEEGYYIRSGIVVIQKNATIKDGTVV

SEQ ID NO: 31 - protein, 522 aa, Aegilops speltoides
  MISC_FEATURE (1)..(522): AGPase large subunit
MSSMQFSSVLPLEGKACVSPVRREGSACERLKVGGSSSIRHDRASRRMCNGGRGPAATGAQCVLTSDASPADTLVLRTSFRRNYADPNEVAAVILGGGTGTQLFPLTSTRATPAVPIGGCYRLIDIPMSNCFNSGINKIFVMTQFNSASLNRHIHRTYLGGGINFTDGSVEVLAATQMPGEAAGWFRGTADAVRKFIWVLEDYYKNKSIEHILILSGDQLYRMDYMELVQKHVDDNADITLSCAPVGESRASEYGLVKFDSSGRVVQFSEKPKGADLEAMKVDTSFLNFAIDDTDKYPYIASMGVYVFKRDVLLNLLKSRYAELHDFGSEILPRALHDHNVQAYVFTDYWEDIGTIRSFFDANMALCEQPPKFEFYDPKTPFFTSPRYLPPTKSDKCRIKEAIISHGCFLRECKIEHSIIGVRSRLNSGSELKNAMMMGADSYETEDEISRLMSEGKVPIGVGENTKISNCIIDMNARIGRDVVISNKEGVQEADRPEEGYYIRSGIVVIQKNATIKDGTNL

SEQ ID NO: 32 - protein, 519 aa, Zea mays
  MISC_FEATURE (1)..(519): AGPase large subunit
MQFSSVLPLEGKACMSPVRRGSGGYGSERMRINCCSIRRNKALRRMCFSARGAVSSTQCVLTSDAGPDTLVVRTSFRRNYADPNEVAAVILGGGTGTQLFPLTSTRATPAVPIGGCYRLIDIPMSNCFNSGINKIFVMTQFNSASLNRHIHRTYLGGGINFTDGSVEVLAATQMPGEAAGWFQGTADAVRKFIWVLEDYYKHKAIEHILILSGDQLYRMDYMELVQKHVDDNADITLSCAPVGERSRASDYGLVKFDSSGRVIQFSEKPKGAALEEMKVDTSFLNFAIDSPAEYPYIASMGVYVFKRDVLLDLLKSRYAELHDFGSEILPKALHEHNVQAYVFTDYWEDIGTIRSFFDANMALCEQPPKFEFYDPKTPFFTSPRYLPPTKSDKCRIKDAIISHGCFLRECAIEHSIVGVRSRLNSGCELKNTMMMGADLYETEDEISRLLAEGKVPIGVGENTKISNCIIDMNARVGRNVSITNKEGVQEADRPDEGYYIRSGIVVVLKNATIKDGTVI

SEQ ID NO: 33 - protein, 522 aa, Aegilops sharonensis
  MISC_FEATURE (1)..(522): AGPase large subunit
MSSMQFSSVLPLEGKACVSPVRREGSASERLKVGDSSSIRHERASRRMCNGGRGPAATGAQCVLTSDASPADTLVLRTSFRRNYADPNEVAAVILGGGTGTQLFPLTSTRATPAVPIGGCYRLIDIPMSNCFNSGINKIFVMTQFNSASLNRHIHRTYLGGGINFTDGSVEVLAATQMPGEAAGWFRGTADAVRKFIWVLEDYYKNKSIEHILILSGDQLYRMDYMELVQKHVDDNADITLSCAPVGESRASEYGLVKFDSSGRVVQFSEKPKGADLEAMKVDTSFLNFAIDDPAKYPYIASMGVYVFKRDVLLNLLKSRYAELHDFGSEILPRALHDHNVQAYVFTDYWEDIGTIRSFFDANMALCEQPPKFEFYDPKTPFFTSPRYLSPTKSDKCRIKEAIISHGCFLRECKIEHSIIGVRSRLNSGSELKNAMMMGADSYETEDEISRLMSEGKVPIGVGENTKISNCIIDMNARIGRDVVISNKEGVQEADRPEEGYYIRSGIVVIQKNATIKDGTNL

SEQ ID NO: 34 - protein, 522 aa, Triticum urartu
  MISC_FEATURE (1)..(522): AGPase large subunit
MSSMQFSSVLPLEGKACISPVRREGSASERLKVGDSSSIRHERASRRMCNGGRGPAATGAQCVLTSDASPADTLVLRTSFRRNYADPNEVAAVILGGGTGTQLFPLTSTRATPAVPIGGCYRLIDIPMSNCFNSGINKIFVMTQFNSASLNRHIHRTYLGGGINFTDGSVEVLAATQMPGEAAGWFRGTADAVRKFIWVLEDYYKNKSIEHILILSGDQLYRMDYMELVQKHVDDNADITLSCAPVGESRASEYGLVKFDSSGRVVQFSEKPKGDDLEAMKVDTSFLNFAIDDPAKYPYIASMGVYVFKRDVLLNLLKSRYAELHDFGSEILPRALHDHNVQAYVFTDYWEDIGTIRSFFDANMALCEQPPKFEFYDPKTPFFTSPRYLPPTKSDKSRIKEAIISHGCFLRECKIEHSIIGVRSRLNSGSELKNAMMMGADSYETEDEISRLMSEGKVPIGVGENTKISNCIIDMNARIGRDVVISNKEGVQEADRPEEGYYIRSGIVVIQKNATIKDGTVV

SEQ ID NO: 35 - protein, 522 aa, Aegilops tauschii
  MISC_FEATURE (1)..(522): AGPase large subunit
MSSMQFSSVLPLEGKACISPVRREGSASERLKVGDSSSIRHERASRRMCNGGRGPAATGAQCVLTSDASPADTLVLRTSFRRNYADPNEVAAVILGGGTGTQLFPLTSTRATPAVPIGGCYRLIDIPMSNCFNSGINKIFVMTQFNSASLNRHIHRTYLGGGINFTDGSVEVLAATQMPGEAAGWFRGTADAVRKFIWVLEDYYKNKSIEHILILSGDQLYRMDYMELVQKHVDDNADITLSCAPVGESRASEYGLVKFDSSGRVVQFSEKPKGDDLEAMKVDTSFLNFAIDDPAKYPYIASMGVYVFKRDVLLNLLKSRYAELHDFGSEILPRALHDHNVQAYVFTDYWEDIGTIRSFFDANMALCEQPPKFEFYDPKTPFFTSPRYLPPTKSDKCRIKEAIISHGCFLRECKIEHSIIGVRSRLNSGSELKNAMMMGADSYETEDEISRLMSEGKVPIGVGENTKISNCIIDMNARIGRDVVISNKEGVQEADRPEEGYYIGSGIVVIQKNATIKDGTVV

SEQ ID NO: 36 - protein, 523 aa, Hordeum vulgare
  MISC_FEATURE (1)..(523): AGPase large subunit
MSSMQFSSVLPLEGKACVSPVRREGSACERLKIGDSSSIRHERASRRMCNGGARGPAATGAQCVLTSDASPADTLVLRTSFRRNYADPNEVAAVILGGGTGTQLFPLTSTRATPAVPIGGCYRLIDIPMSNCFNSGINKIFVMTQFNSASLNRHIHRTYLGGGINFTDGSVEVLAATQMPGEAAGWFRGTADAVRKFIWVLEDYYKHKSIEHILILSGDQLYRMDYMELVQKHVDDNADITLSCAPVGESRASEYGLVKFDSSRRVIQFSEKPKGDDLEAMKVDTSFLNFAIDDPAKYPYIASMGVYVFKRDVLLNLLKSRYAELHDFGSEILPRALHDHNVQAYVFTDYWEDIGTIRSFFDANMALCEQPPKFEFYDPKTPFFTSPRYLPPTKSDKCRIKEAIISHGCFLRECKIEHSIIGVRSRLNSGSELKNAMMMGADSYETEDEISRLMSEGKVPIGVGENTKISNCIIDMNARIGRDVVISNKEGVQEADRPEEGYYIRSGIVVIQKNATIKDGTVV

SEQ ID NO: 37 - protein, 522 aa, Triticum aestivum
  MISC_FEATURE (1)..(522): AGPase large subunit
MSSMQFSSVLPLEGKACVSPVRREGSACERLKVGDSSSIRHDRASRRMCNGGRGPAATGAQCVLTSDASPADTLVLRTSFRRNYADPNEVAAVILGGGTGTQLFPLTSTRATPAVPIGGCYRLIDIPMSNCFNSGINKIFVMTQFNSASLNRHIHRTYLGGGINFTDGSVEVLAATQMPGEAAGWFRGTADAVRKFIWVLEDYYKNKSIEHILILSGDQLYRMDYMELVQKHVDDNADITLSCAPVGESRASEYGLVKFDSSGRVVQFSEKPKGDDLEAMKVDTSFLNFAIDDPAKYPYIASMGVYVFKRDVLLNLLKSRYAELHDFGSEILPRALHDHNVQAYVFTDYWEDIGQSDPSFDANMALCEQPPKFEFYDPKTPFFTSPRYLPPTKSDKCRIKEAIISHGCFLRECKIEHSIIGVRSRLNSGSELKNAMMMGADSYETEDEISRLMSEGKVPIGVGENTKISNCIIDMNARIGRDVVISNKEGVQEADRPEEGYYIRSGIVVIQKNATIKDGTVV

SEQ ID NO: 38 - protein, 527 aa, Hordeum vulgare
  MISC_FEATURE (1)..(527): AGPase large subunit
MSSMQFSSVLPLEGKACVSPVRREGSACERLKIGDSSSIRHERASRRMCNGGAGAPPPPVRSACSPPTPARPTPLFSGRPSGGITPIRTRSRPSVAAVILGGGTGTQLFPLTSTRATPAVPIGGCYRLIDIPMSNCFNSGINKIFVMTQFNSASLNRHIHRTYLGGGINFTDGSVEVLAATQMPGEAAGWFRGTADAVRKFIWVLEDYYKHKSIEHILILSGDQLYRMDYMELVQKHVDDNADITLSCAPVGESRASEYGLVKFDSSGRVIQFSEKPKGDDLEAMKVDTSFLNFAIDDPAKYPYIASMGVYVFKRDVLLNLLKSRYAELHDFGSEILPRALHDHNVQAYVFTDYWEDIGTIRSFFDANMALCEQPPKFEFYDPKTPFFTSPRYLPPTKSDKCRIKEAIISHGCFLRECKIEHSIIGVRSRLNSGSELKNAMMMGADSYETEDEISRLMSEGKVPIGVGENTKISNCIIDMNARIGRDVVISNKEGVQEADRPEEGYYIRSGIVVIQKNATIKDGTVV

SEQ ID NO: 39 - protein, 525 aa, Phoenix dactylifera
  MISC_FEATURE (1)..(525): AGPase large subunit
MVGPVMALGSAGLKANAYLGQVKKGGVGSGESGIWGEELCSRLATKCVWNRSLEGRRKAGVTFSILTTDVSKESMVFQTPMFGTQKADPKSVASIILGGGAGTQLFPLTSTRATPAVPVGGCYRLIDIPMSNCINSGINKIFITTQFNSASLNRHIHRTYNLGSGINFGDGFIEVLAATQTPGEAGMNWFQGTADAVRQFIWVFEDNRNKNIEHILILSGDHLYRMDYMEFMQKHIDTGADITVSCVPVGDSRASDYGLMKIDKTGRIIQFSEKPKGADLEAMKVDTTFLRLSHQDAKRYPYIASMGVYVFRRDVLLKLLRWRYPKSNDFGSEILPSSVKDYNVQAYLFNDYWEDIGTIKPFFDANLALTDQPPKFQFYDPKTPFFTSPRFLPPTKIEKCRVLDAIISHGCFLRECSVQHSIVGVRSRLDYGAELKDTVMMGADNYETEAEIASLLAEGKVPIGVGRNTKISNCIIDMNVRIGKNIVIANKDGVQEADRPHEGFYIRSGITIILKNATIKDGTII

SEQ ID NO: 40 - protein, 520 aa, Elaeis guineensis
  MISC_FEATURE (1)..(520): AGPase large subunit
MDVGCVGLKANVYLGHAKKGGVGSGGSGIWGEGFCTRVAKKCVESRSLERGGKAGVTVSILTSDVSKETMVFQTPMFGSQKADPKNVASIILGGGAGTQLFPLTSTRATPAVPVGGCYRLIDIPMSNCINSGINKIFIMTQFNSASLNRHIHRTYNLGNGINFGDGFIEVLAATQTPGEFGMNWFQGTADAVRQFIWVFEDNRNKNIEHILILSGDHLYRMDYMDFMQKHIDTGADITVSCVPVGDSRASDYGLMKIDKTGRIIQFSEKPQGADLEAMKVDTTFLRLSSQDAKRYPYIASMGVYIFRRDVLLKLLRWRYPKSNDFGSEILPSSLQDHNVQAYLFNDYWEDIGTIKSFFDANLALTEQPPKFQFYDPMTPFFTSPRFLPPTKIEKCRVLDAIISHGCFLRECSVQHSIVGVRSRLDYGAELKDTVMMGADYYETEAEIASLLAEGKVPIGVGQNTKISNCIIDMNVRIGKNVIIANKEGVQEADRPCEGFYIRSGITIILKNAIIKDGTII

SEQ ID NO: 41 - protein, 529 aa, Musa acuminata
  MISC_FEATURE (1)..(529): AGPase large subunit
MDVCCVGSMANACFSQVKRASVGCRESGIWGDGIGNGPKTTKVWDSKVAKSLKTRSSFGGRKTGVAFSVLTSDVDQETMVFHAPMFGRRTADPKSVASIILGGGAGTQLVPLTSTRATPAVPIGGCYRLIDIPMSNCINSGINKIFIMTQFNSASLNRHISRTYNFGNGINFGDGFVEVLAATQTPGEAGMNWFQGTADAVRQFTWVFEDNKNKNIEHILILSGDQLYRMDYMDLVQKHVDTGADITVSCVPVGHSRASDYGLVKIDEAGRIIQFFEKPKGADLEAMNDNGTFLRLSHQDAMKYPYIASMGVYVFKRDVLLKLLRWNYPKCNDFGSEILPSAVEEHNVQAYAFSDYWEDIGTIRSFFDANLALTEQPPKFQFYDPMTPFFTSPRFLPPTKIEKCRVMDAIISHGCFLRECSVERSIVGVRSRLDFGVELKDTMMMGADIYETEAEISSHLADDKVPIGVGQKTKIRNCVIDMNARIGKNVVIANKDGVQEADRASEGFYVRSGIVVILKNATIKDGTVI

SEQ ID NO: 42 - protein, 528 aa, Nelumbo nucifera
  MISC_FEATURE (1)..(528): AGPase large subunit
MDSCCATLKANAHPVQVRKGLITNGDNGFWGECLRGSLNNGTMGTKLTKSLRTENGGRKIKTGVAYSVFTDVNKEVATQTFQAPLLGKQIADPKNVASIILGGGAGTRLFPLTSRRAKPAVPIGGCYRLIDVPMSNCINSGINKIFIMTQFNSASLNRHLARTYNFGNGVNFGDGFVEVLAATQTPETGTRWFQGTADAVRQFIWVFEDAKNKHVENILILSGDHLYRMEYMDFVQKHVDTNADITVSCVPMDDSRASDYGLMKIDNTGRIIQFLEKPKGADLKAMQVNTTLLGLSQQEAMKYPYIASMGVYVFRTEVLLKLLRWRYPTCNDFGSEIIPAAVKEHNVQAYLFNDYWEDIGTIKSFFDANLALTEQPPNFEFYDPKTPFFTSPRFLPPTKVDKCRIVNAIISHGCFLRECSVQHSIVGVRSRLEYGCELKDTMMMGADYYHTESEIASLLAEGKVPIGVGQNTKIRNCIIDKNAKIGRDVIIANKDGVQEADRPDDGFYIRSGIIVILKNATIKDGTVI

SEQ ID NO: 43 - DNA, 2019 bp, Zea mays
  misc_feature (1)..(2019): modified maize sh2 cDNA
  misc_feature (745)..(747): T249K modification
  misc_feature (754)..(756): Q252G modification
atgggcgggcagtgctgtgagttagagcagtgtggcaatgttggtggtagtggtgacacccttcgggagcggaaggaggctggaacttcagattatttggcattctgtaaaagacccaacacccaaatgcttgaaaaaatatatcatgttgtaaaaattcgatctgcatttccttctgcaaactcttctggaagaaaggtgtctaaaactatgaacgggttacagaaagcaaaggccaagggcaaaattggaacactaatgatggttggttgcatgagtctgtcgattacttgcaagaaatgtgaacctttgtttctttcatctccaccagtatgccctctcatactctatataaacacatcaacagcatcgcaattagccacaagatcacttcgggaggcaagtgcgatttcgatctcgcagccaccttttttttgttctgttgtgtatctagtagttggaggagatatgcagtttgcacttgcattggacacgaactcaggtcctcaccagataagatcttgtgagggtgatgggattgacaggttggaaaaattaagtattgggggcagaaagcaggagaaagctttgagaaataggtgctttggtggtagagttgctgcaactacacaatgtattcttacctcagatgcttgtcctgaaactcttcattctcaaacacagtcctctaggaaaaattatgctgatgcaaaccgtgtatctgctatcattttgggcggaggcaaaggatctgggctctttcctctgacaagcacaagagctacgcctgctgtacctgttggaggatgttacaggcttattgatatccctatgagtaactgcttcaacagtggtataaataagatatttgtgatgagtcagttcaattctacttcgcttaaccgccatattcatcgtacataccttgaaggcgggatcaactttgctgatggatctgtacaggtattagcggctacacaaatgcctgaagagccagctggatggttccagggtacagcagactctatcagaaaatttatctgggtactcgaggattattacagtcacaaatccattgacaacattgtaatcttgagtggcgatcagctttatcggatgaattacatggaacttgtgcagaaacatgtcgaggacgatgctgatatcactatatcatgtgctcctgttgatgagagccgagcttctaaaaatgggctagtgaagattgatcatactggacgtgtacttcaattctttgaaaaaccaaagggtgctgatttgaattctatgagagttgagaccaacttcctgagctatgctatagatgatgcacagaaatatccataccttgcatcaatgggcatttatgtcttcaagaaagatgcacttttagaccttctcaagtcaaaatatattcaattacatgactttggatctgaaatcctcccaagagctgtactagatcatagtgtgcaggcatgcatttttacgggctattgggaggatgttggaacaatcaaatcattctttgatgcaaacttggccctcactgagcagccttccaagtttgatttttatgatccaaaaacacctttcttcactgcaccccgatgcttgcctccgacgcaattggacaagtgcaagatgaaagatgcatttatctcagatggttgcttactgagagaatgcaacatcgagcattctgtgattggagtctgctcacgtgtcagctctggatgtgaactcaaggactccgtgatgatgggagcggacacctatgaaactgaagaagaagcttcaaagctactgttagctgggaaggtcccagttggaataggaaggaacacaaagataaggaactgtatcattgacatgaatgctaggattgggaagaacgtggtgatcacaaacagtaagggcatccaagaggctgatcacccggaagaagggtactacataaggtctggaatcgtggtgatcttgaagaatgcaaccatcaacgatgggtctgtcatatag

SEQ ID NO: 44 - DNA, 2019 bp, Zea mays
  misc_feature (1)..(2019): modified maize sh2 cDNA
  misc_feature (934)..(936): G312N modification
  misc_feature (949)..(951): D317G modification
atgggcgggcagtgctgtgagttagagcagtgtggcaatgttggtggtagtggtgacacccttcgggagcggaaggaggctggaacttcagattatttggcattctgtaaaagacccaacacccaaatgcttgaaaaaatatatcatgttgtaaaaattcgatctgcatttccttctgcaaactcttctggaagaaaggtgtctaaaactatgaacgggttacagaaagcaaaggccaagggcaaaattggaacactaatgatggttggttgcatgagtctgtcgattacttgcaagaaatgtgaacctttgtttctttcatctccaccagtatgccctctcatactctatataaacacatcaacagcatcgcaattagccacaagatcacttcgggaggcaagtgcgatttcgatctcgcagccaccttttttttgttctgttgtgtatctagtagttggaggagatatgcagtttgcacttgcattggacacgaactcaggtcctcaccagataagatcttgtgagggtgatgggattgacaggttggaaaaattaagtattgggggcagaaagcaggagaaagctttgagaaataggtgctttggtggtagagttgctgcaactacacaatgtattcttacctcagatgcttgtcctgaaactcttcattctcaaacacagtcctctaggaaaaattatgctgatgcaaaccgtgtatctgctatcattttgggcggaggcactggatctcagctctttcctctgacaagcacaagagctacgcctgctgtacctgttggaggatgttacaggcttattgatatccctatgagtaactgcttcaacagtggtataaataagatatttgtgatgagtcagttcaattctacttcgcttaaccgccatattcatcgtacataccttgaaggcaacatcaactttgctggtggatctgtacaggtattagcggctacacaaatgcctgaagagccagctggatggttccagggtacagcagactctatcagaaaatttatctgggtactcgaggattattacagtcacaaatccattgacaacattgtaatcttgagtggcgatcagctttatcggatgaattacatggaacttgtgcagaaacatgtcgaggacgatgctgatatcactatatcatgtgctcctgttgatgagagccgagcttctaaaaatgggctagtgaagattgatcatactggacgtgtacttcaattctttgaaaaaccaaagggtgctgatttgaattctatgagagttgagaccaacttcctgagctatgctatagatgatgcacagaaatatccataccttgcatcaatgggcatttatgtcttcaagaaagatgcacttttagaccttctcaagtcaaaatatattcaattacatgactttggatctgaaatcctcccaagagctgtactagatcatagtgtgcaggcatgcatttttacgggctattgggaggatgttggaacaatcaaatcattctttgatgcaaacttggccctcactgagcagccttccaagtttgatttttatgatccaaaaacacctttcttcactgcaccccgatgcttgcctccgacgcaattggacaagtgcaagatgaaagatgcatttatctcagatggttgcttactgagagaatgcaacatcgagcattctgtgattggagtctgctcacgtgtcagctctggatgtgaactcaaggactccgtgatgatgggagcggacacctatgaaactgaagaagaagcttcaaagctactgttagctgggaaggtcccagttggaataggaaggaacacaaagataaggaactgtatcattgacatgaatgctaggattgggaagaacgtggtgatcacaaacagtaagggcatccaagaggctgatcacccggaagaagggtactacataaggtctggaatcgtggtgatcttgaagaatgcaaccatcaacgatgggtctgtcatatag

SEQ ID NO: 45 - DNA, 2019 bp, Zea mays
  misc_feature (1)..(2019): modified maize sh2 cDNA
  misc_feature (745)..(747): T249K modification
  misc_feature (754)..(756): Q252G modification
  misc_feature (934)..(936): G312N modification
  misc_feature (949)..(951): D317G modification
atgggcgggcagtgctgtgagttagagcagtgtggcaatgttggtggtagtggtgacacccttcgggagcggaaggaggctggaacttcagattatttggcattctgtaaaagacccaacacccaaatgcttgaaaaaatatatcatgttgtaaaaattcgatctgcatttccttctgcaaactcttctggaagaaaggtgtctaaaactatgaacgggttacagaaagcaaaggccaagggcaaaattggaacactaatgatggttggttgcatgagtctgtcgattacttgcaagaaatgtgaacctttgtttctttcatctccaccagtatgccctctcatactctatataaacacatcaacagcatcgcaattagccacaagatcacttcgggaggcaagtgcgatttcgatctcgcagccaccttttttttgttctgttgtgtatctagtagttggaggagatatgcagtttgcacttgcattggacacgaactcaggtcctcaccagataagatcttgtgagggtgatgggattgacaggttggaaaaattaagtattgggggcagaaagcaggagaaagctttgagaaataggtgctttggtggtagagttgctgcaactacacaatgtattcttacctcagatgcttgtcctgaaactcttcattctcaaacacagtcctctaggaaaaattatgctgatgcaaaccgtgtatctgctatcattttgggcggaggcaaaggatctgggctctttcctctgacaagcacaagagctacgcctgctgtacctgttggaggatgttacaggcttattgatatccctatgagtaactgcttcaacagtggtataaataagatatttgtgatgagtcagttcaattctacttcgcttaaccgccatattcatcgtacataccttgaaggcaacatcaactttgctggtggatctgtacaggtattagcggctacacaaatgcctgaagagccagctggatggttccagggtacagcagactctatcagaaaatttatctgggtactcgaggattattacagtcacaaatccattgacaacattgtaatcttgagtggcgatcagctttatcggatgaattacatggaacttgtgcagaaacatgtcgaggacgatgctgatatcactatatcatgtgctcctgttgatgagagccgagcttctaaaaatgggctagtgaagattgatcatactggacgtgtacttcaattctttgaaaaaccaaagggtgctgatttgaattctatgagagttgagaccaacttcctgagctatgctatagatgatgcacagaaatatccataccttgcatcaatgggcatttatgtcttcaagaaagatgcacttttagaccttctcaagtcaaaatatattcaattacatgactttggatctgaaatcctcccaagagctgtactagatcatagtgtgcaggcatgcatttttacgggctattgggaggatgttggaacaatcaaatcattctttgatgcaaacttggccctcactgagcagccttccaagtttgatttttatgatccaaaaacacctttcttcactgcaccccgatgcttgcctccgacgcaattggacaagtgcaagatgaaagatgcatttatctcagatggttgcttactgagagaatgcaacatcgagcattctgtgattggagtctgctcacgtgtcagctctggatgtgaactcaaggactccgtgatgatgggagcggacacctatgaaactgaagaagaagcttcaaagctactgttagctgggaaggtcccagttggaataggaaggaacacaaagataaggaactgtatcattgacatgaatgctaggattgggaagaacgtggtgatcacaaacagtaagggcatccaagaggctgatcacccggaagaagggtactacataaggtctggaatcgtggtgatcttgaagaatgcaaccatcaacgatgggtctgtcatatag

SEQ ID NO: 46 - DNA, 2019 bp, Zea mays
  misc_feature (1)..(2019): modified maize sh2 cDNA
  misc_feature (745)..(747): T249K modification
  misc_feature (754)..(756): Q252G modification
  misc_feature (934)..(936): G312N modification
  misc_feature (949)..(951): D317G modification
  misc_feature (1795)..(1797): A599R modification
atgggcgggcagtgctgtgagttagagcagtgtggcaatgttggtggtagtggtgacacccttcgggagcggaaggaggctggaacttcagattatttggcattctgtaaaagacccaacacccaaatgcttgaaaaaatatatcatgttgtaaaaattcgatctgcatttccttctgcaaactcttctggaagaaaggtgtctaaaactatgaacgggttacagaaagcaaaggccaagggcaaaattggaacactaatgatggttggttgcatgagtctgtcgattacttgcaagaaatgtgaacctttgtttctttcatctccaccagtatgccctctcatactctatataaacacatcaacagcatcgcaattagccacaagatcacttcgggaggcaagtgcgatttcgatctcgcagccaccttttttttgttctgttgtgtatctagtagttggaggagatatgcagtttgcacttgcattggacacgaactcaggtcctcaccagataagatcttgtgagggtgatgggattgacaggttggaaaaattaagtattgggggcagaaagcaggagaaagctttgagaaataggtgctttggtggtagagttgctgcaactacacaatgtattcttacctcagatgcttgtcctgaaactcttcattctcaaacacagtcctctaggaaaaattatgctgatgcaaaccgtgtatctgctatcattttgggcggaggcaaaggatctgggctctttcctctgacaagcacaagagctacgcctgctgtacctgttggaggatgttacaggcttattgatatccctatgagtaactgcttcaacagtggtataaataagatatttgtgatgagtcagttcaattctacttcgcttaaccgccatattcatcgtacataccttgaaggcaacatcaactttgctggtggatctgtacaggtattagcggctacacaaatgcctgaagagccagctggatggttccagggtacagcagactctatcagaaaatttatctgggtactcgaggattattacagtcacaaatccattgacaacattgtaatcttgagtggcgatcagctttatcggatgaattacatggaacttgtgcagaaacatgtcgaggacgatgctgatatcactatatcatgtgctcctgttgatgagagccgagcttctaaaaatgggctagtgaagattgatcatactggacgtgtacttcaattctttgaaaaaccaaagggtgctgatttgaattctatgagagttgagaccaacttcctgagctatgctatagatgatgcacagaaatatccataccttgcatcaatgggcatttatgtcttcaagaaagatgcacttttagaccttctcaagtcaaaatatattcaattacatgactttggatctgaaatcctcccaagagctgtactagatcatagtgtgcaggcatgcatttttacgggctattgggaggatgttggaacaatcaaatcattctttgatgcaaacttggccctcactgagcagccttccaagtttgatttttatgatccaaaaacacctttcttcactgcaccccgatgcttgcctccgacgcaattggacaagtgcaagatgaaagatgcatttatctcagatggttgcttactgagagaatgcaacatcgagcattctgtgattggagtctgctcacgtgtcagctctggatgtgaactcaaggactccgtgatgatgggagcggacacctatgaaactgaagaagaacgttcaaagctactgttagctgggaaggtcccagttggaataggaaggaacacaaagataaggaactgtatcattgacatgaatgctaggattgggaagaacgtggtgatcacaaacagtaagggcatccaagaggctgatcacccggaagaagggtactacataaggtctggaatcgtggtgatcttgaagaatgcaaccatcaacgatgggtctgtcatatag

SEQ ID NO: 47 - DNA, 2019 bp, Zea mays
  misc_feature (1)..(2019): modified maize sh2 cDNA
  misc_feature (745)..(747): T249K modification
  misc_feature (754)..(756): Q252G modification
  misc_feature (1795)..(1797): A599R modification
atgggcgggcagtgctgtgagttagagcagtgtggcaatgttggtggtagtggtgacacccttcgggagcggaaggaggctggaacttcagattatttggcattctgtaaaagacccaacacccaaatgcttgaaaaaatatatcatgttgtaaaaattcgatctgcatttccttctgcaaactcttctggaagaaaggtgtctaaaactatgaacgggttacagaaagcaaaggccaagggcaaaattggaacactaatgatggttggttgcatgagtctgtcgattacttgcaagaaatgtgaacctttgtttctttcatctccaccagtatgccctctcatactctatataaacacatcaacagcatcgcaattagccacaagatcacttcgggaggcaagtgcgatttcgatctcgcagccaccttttttttgttctgttgtgtatctagtagttggaggagatatgcagtttgcacttgcattggacacgaactcaggtcctcaccagataagatcttgtgagggtgatgggattgacaggttggaaaaattaagtattgggggcagaaagcaggagaaagctttgagaaataggtgctttggtggtagagttgctgcaactacacaatgtattcttacctcagatgcttgtcctgaaactcttcattctcaaacacagtcctctaggaaaaattatgctgatgcaaaccgtgtatctgctatcattttgggcggaggcaaaggatctgggctctttcctctgacaagcacaagagctacgcctgctgtacctgttggaggatgttacaggcttattgatatccctatgagtaactgcttcaacagtggtataaataagatatttgtgatgagtcagttcaattctacttcgcttaaccgccatattcatcgtacataccttgaaggcgggatcaactttgctgatggatctgtacaggtattagcggctacacaaatgcctgaagagccagctggatggttccagggtacagcagactctatcagaaaatttatctgggtactcgaggattattacagtcacaaatccattgacaacattgtaatcttgagtggcgatcagctttatcggatgaattacatggaacttgtgcagaaacatgtcgaggacgatgctgatatcactatatcatgtgctcctgttgatgagagccgagcttctaaaaatgggctagtgaagattgatcatactggacgtgtacttcaattctttgaaaaaccaaagggtgctgatttgaattctatgagagttgagaccaacttcctgagctatgctatagatgatgcacagaaatatccataccttgcatcaatgggcatttatgtcttcaagaaagatgcacttttagaccttctcaagtcaaaatatattcaattacatgactttggatctgaaatcctcccaagagctgtactagatcatagtgtgcaggcatgcatttttacgggctattgggaggatgttggaacaatcaaatcattctttgatgcaaacttggccctcactgagcagccttccaagtttgatttttatgatccaaaaacacctttcttcactgcaccccgatgcttgcctccgacgcaattggacaagtgcaagatgaaagatgcatttatctcagatggttgcttactgagagaatgcaacatcgagcattctgtgattggagtctgctcacgtgtcagctctggatgtgaactcaaggactccgtgatgatgggagcggacacctatgaaactgaagaagaacgttcaaagctactgttagctgggaaggtcccagttggaataggaaggaacacaaagataaggaactgtatcattgacatgaatgctaggattgggaagaacgtggtgatcacaaacagtaagggcatccaagaggctgatcacccggaagaagggtactacataaggtctggaatcgtggtgatcttgaagaatgcaaccatcaacgatgggtctgtcatatag

SEQ ID NO: 48 - DNA, 2019 bp, Zea mays
  misc_feature (1)..(2019): modified maize sh2 cDNA
  misc_feature (934)..(936): G312N modification
  misc_feature (949)..(951): D317G modification
  misc_feature (1795)..(1797): A599R modification
atgggcgggcagtgctgtgagttagagcagtgtggcaatgttggtggtagtggtgacacccttcgggagcggaaggaggctggaacttcagattatttggcattctgtaaaagacccaacacccaaatgcttgaaaaaatatatcatgttgtaaaaattcgatctgcatttccttctgcaaactcttctggaagaaaggtgtctaaaactatgaacgggttacagaaagcaaaggccaagggcaaaattggaacactaatgatggttggttgcatgagtctgtcgattacttgcaagaaatgtgaacctttgtttctttcatctccaccagtatgccctctcatactctatataaacacatcaacagcatcgcaattagccacaagatcacttcgggaggcaagtgcgatttcgatctcgcagccaccttttttttgttctgttgtgtatctagtagttggaggagatatgcagtttgcacttgcattggacacgaactcaggtcctcaccagataagatcttgtgagggtgatgggattgacaggttggaaaaattaagtattgggggcagaaagcaggagaaagctttgagaaataggtgctttggtggtagagttgctgcaactacacaatgtattcttacctcagatgcttgtcctgaaactcttcattctcaaacacagtcctctaggaaaaattatgctgatgcaaaccgtgtatctgctatcattttgggcggaggcactggatctcagctctttcctctgacaagcacaagagctacgcctgctgtacctgttggaggatgttacaggcttattgatatccctatgagtaactgcttcaacagtggtataaataagatatttgtgatgagtcagttcaattctacttcgcttaaccgccatattcatcgtacataccttgaaggcaacatcaactttgctggtggatctgtacaggtattagcggctacacaaatgcctgaagagccagctggatggttccagggtacagcagactctatcagaaaatttatctgggtactcgaggattattacagtcacaaatccattgacaacattgtaatcttgagtggcgatcagctttatcggatgaattacatggaacttgtgcagaaacatgtcgaggacgatgctgatatcactatatcatgtgctcctgttgatgagagccgagcttctaaaaatgggctagtgaagattgatcatactggacgtgtacttcaattctttgaaaaaccaaagggtgctgatttgaattctatgagagttgagaccaacttcctgagctatgctatagatgatgcacagaaatatccataccttgcatcaatgggcatttatgtcttcaagaaagatgcacttttagaccttctcaagtcaaaatatattcaattacatgactttggatctgaaatcctcccaagagctgtactagatcatagtgtgcaggcatgcatttttacgggctattgggaggatgttggaacaatcaaatcattctttgatgcaaacttggccctcactgagcagccttccaagtttgatttttatgatccaaaaacacctttcttcactgcaccccgatgcttgcctccgacgcaattggacaagtgcaagatgaaagatgcatttatctcagatggttgcttactgagagaatgcaacatcgagcattctgtgattggagtctgctcacgtgtcagctctggatgtgaactcaaggactccgtgatgatgggagcggacacctatgaaactgaagaagaacgttcaaagctactgttagctgggaaggtcccagttggaataggaaggaacacaaagataaggaactgtatcattgacatgaatgctaggattgggaagaacgtggtgatcacaaacagtaagggcatccaagaggctgatcacccggaagaagggtactacataaggtctggaatcgtggtgatcttgaagaatgcaaccatcaacgatgggtctgtcatatag

SEQ ID NO: 49 - protein, 518 aa, Oryza sativa
  MISC_FEATURE (1)..(518): modified Oryza sativa AGPase large subunit protein
  MISC_FEATURE (95)..(95): T95K
  MISC_FEATURE (98)..(98): Q98G
MQFMMPLDTNACAQPMRRAGEGAGTERLMERLNIGGMTQEKALRKRCFGDGVTGTARCVFTSDADRDTPHLRTQSSRKNYADASHVSAVILGGGKGVGLFPLTSTRATPAVPVGGCYRLIDIPMSNCFNSGINKIFVMTQFNSASLNRHIHHTYLGGGINFTDGSVQVLAATQMPDEPAGWFQGTADAIRKFMWILEDHYNQNNIEHVVILCGDQLYRMNYMELVQKHVDDNADITISCAPIDGSRASDYGLVKFDDSGRVIQFLEKPEGADLESMKVDTSFLSYAIDDKQKYPYIASMGIYVLKKDVLLDILKSKYAHLQDFGSEILPRAVLEHNVKACVFTEYWEDIGTIKSFFDANLALTEQPPKFEFYDPKTPFFTSPRYLPPARLEKCKIKDAIISDGCSFSECTIEHSVIGISSRVSIGCELKDTMMMGADQYETEEETSKLLFEGKVPIGIGENTKIRNCIIDMNARIGRNVIIANTQGVQESDHPEEGYYIRSGIVVILKNATIKDGTVI

SEQ ID NO: 50 - protein, 518 aa, Oryza sativa
  MISC_FEATURE (1)..(518): Modified Oryza sativa AGPase large subunit protein
  MISC_FEATURE (158)..(158): G158N
  MISC_FEATURE (163)..(163): D163G
MQFMMPLDTNACAQPMRRAGEGAGTERLMERLNIGGMTQEKALRKRCFGDGVTGTARCVFTSDADRDTPHLRTQSSRKNYADASHVSAVILGGGTGVQLFPLTSTRATPAVPVGGCYRLIDIPMSNCFNSGINKIFVMTQFNSASLNRHIHHTYLGGNINFTGGSVQVLAATQMPDEPAGWFQGTADAIRKFMWILEDHYNQNNIEHVVILCGDQLYRMNYMELVQKHVDDNADITISCAPIDGSRASDYGLVKFDDSGRVIQFLEKPEGADLESMKVDTSFLSYAIDDKQKYPYIASMGIYVLKKDVLLDILKSKYAHLQDFGSEILPRAVLEHNVKACVFTEYWEDIGTIKSFFDANLALTEQPPKFEFYDPKTPFFTSPRYLPPARLEKCKIKDAIISDGCSFSECTIEHSVIGISSRVSIGCELKDTMMMGADQYETEEETSKLLFEGKVPIGIGENTKIRNCIIDMNARIGRNVIIANTQGVQESDHPEEGYYIRSGIVVILKNATIKDGTVI

SEQ ID NO: 51 - protein, 518 aa, Oryza sativa
  MISC_FEATURE (1)..(518): Modified Oryza sativa AGPase large subunit protein
  MISC_FEATURE (95)..(95): T95K
  MISC_FEATURE (98)..(98): Q98G
  MISC_FEATURE (158)..(158): G158N
  MISC_FEATURE (163)..(163): D163G
MQFMMPLDTNACAQPMRRAGEGAGTERLMERLNIGGMTQEKALRKRCFGDGVTGTARCVFTSDADRDTPHLRTQSSRKNYADASHVSAVILGGGKGVGLFPLTSTRATPAVPVGGCYRLIDIPMSNCFNSGINKIFVMTQFNSASLNRHIHHTYLGGNINFTGGSVQVLAATQMPDEPAGWFQGTADAIRKFMWILEDHYNQNNIEHVVILCGDQLYRMNYMELVQKHVDDNADITISCAPIDGSRASDYGLVKFDDSGRVIQFLEKPEGADLESMKVDTSFLSYAIDDKQKYPYIASMGIYVLKKDVLLDILKSKYAHLQDFGSEILPRAVLEHNVKACVFTEYWEDIGTIKSFFDANLALTEQPPKFEFYDPKTPFFTSPRYLPPARLEKCKIKDAIISDGCSFSECTIEHSVIGISSRVSIGCELKDTMMMGADQYETEEETSKLLFEGKVPIGIGENTKIRNCIIDMNARIGRNVIIANTQGVQESDHPEEGYYIRSGIVVILKNATIKDGTVI

SEQ ID NO: 52 - protein, 518 aa, Oryza sativa
  MISC_FEATURE (1)..(518): Modified Oryza sativa AGPase large subunit protein
  MISC_FEATURE (95)..(95): T95K
  MISC_FEATURE (98)..(98): Q98G
  MISC_FEATURE (158)..(158): G158N
  MISC_FEATURE (163)..(163): D163G
  MISC_FEATURE (445)..(445): T445R
MQFMMPLDTNACAQPMRRAGEGAGTERLMERLNIGGMTQEKALRKRCFGDGVTGTARCVFTSDADRDTPHLRTQSSRKNYADASHVSAVILGGGKGVGLFPLTSTRATPAVPVGGCYRLIDIPMSNCFNSGINKIFVMTQFNSASLNRHIHHTYLGGNINFTGGSVQVLAATQMPDEPAGWFQGTADAIRKFMWILEDHYNQNNIEHVVILCGDQLYRMNYMELVQKHVDDNADITISCAPIDGSRASDYGLVKFDDSGRVIQFLEKPEGADLESMKVDTSFLSYAIDDKQKYPYIASMGIYVLKKDVLLDILKSKYAHLQDFGSEILPRAVLEHNVKACVFTEYWEDIGTIKSFFDANLALTEQPPKFEFYDPKTPFFTSPRYLPPARLEKCKIKDAIISDGCSFSECTIEHSVIGISSRVSIGCELKDTMMMGADQYETEEERSKLLFEGKVPIGIGENTKIRNCIIDMNARIGRNVIIANTQGVQESDHPEEGYYIRSGIVVILKNATIKDGTVI

SEQ ID NO: 53 - protein, 518 aa, Oryza sativa
  MISC_FEATURE (1)..(518): Modified Oryza sativa AGPase large subunit protein
  MISC_FEATURE (95)..(95): T95K
  MISC_FEATURE (98)..(98): Q98G
  MISC_FEATURE (445)..(445): T445R
MQFMMPLDTNACAQPMRRAGEGAGTERLMERLNIGGMTQEKALRKRCFGDGVTGTARCVFTSDADRDTPHLRTQSSRKNYADASHVSAVILGGGKGVGLFPLTSTRATPAVPVGGCYRLIDIPMSNCFNSGINKIFVMTQFNSASLNRHIHHTYLGGGINFTDGSVQVLAATQMPDEPAGWFQGTADAIRKFMWILEDHYNQNNIEHVVILCGDQLYRMNYMELVQKHVDDNADITISCAPIDGSRASDYGLVKFDDSGRVIQFLEKPEGADLESMKVDTSFLSYAIDDKQKYPYIASMGIYVLKKDVLLDILKSKYAHLQDFGSEILPRAVLEHNVKACVFTEYWEDIGTIKSFFDANLALTEQPPKFEFYDPKTPFFTSPRYLPPARLEKCKIKDAIISDGCSFSECTIEHSVIGISSRVSIGCELKDTMMMGADQYETEEERSKLLFEGKVPIGIGENTKIRNCIIDMNARIGRNVIIANTQGVQESDHPEEGYYIRSGIVVILKNATIKDGTVI

SEQ ID NO: 54 - protein, 518 aa, Oryza sativa
  MISC_FEATURE (1)..(518): Modified Oryza sativa AGPase large subunit protein
  MISC_FEATURE (158)..(158): G158N
  MISC_FEATURE (163)..(163): D163G
  MISC_FEATURE (445)..(445): T445R
MQFMMPLDTNACAQPMRRAGEGAGTERLMERLNIGGMTQEKALRKRCFGDGVTGTARCVFTSDADRDTPHLRTQSSRKNYADASHVSAVILGGGTGVQLFPLTSTRATPAVPVGGCYRLIDIPMSNCFNSGINKIFVMTQFNSASLNRHIHHTYLGGNINFTGGSVQVLAATQMPDEPAGWFQGTADAIRKFMWILEDHYNQNNIEHVVILCGDQLYRMNYMELVQKHVDDNADITISCAPIDGSRASDYGLVKFDDSGRVIQFLEKPEGADLESMKVDTSFLSYAIDDKQKYPYIASMGIYVLKKDVLLDILKSKYAHLQDFGSEILPRAVLEHNVKACVFTEYWEDIGTIKSFFDANLALTEQPPKFEFYDPKTPFFTSPRYLPPARLEKCKIKDAIISDGCSFSECTIEHSVIGISSRVSIGCELKDTMMMGADQYETEEERSKLLFEGKVPIGIGENTKIRNCIIDMNARIGRNVIIANTQGVQESDHPEEGYYIRSGIVVILKNATIKDGTVI

SEQ ID NO: 55 - DNA, 4028 bp, Oryza sativa
  misc_feature (1)..(4028): Modified AGPase large subunit gDNA
  misc_feature (675)..(677): T95K mutation
  misc_feature (684)..(686): Q98G mutation
atgcaattcatgatgccattggatacgaatgcttgtgcacaaccaatgagaagggctggtgagggtgctgggactgagaggttgatggaaagattgaatattgggggcatgactcaggagaaagcactgaggaagaggtgctttggtgatggagttactggaactgcacgatgtgtgttcacatcagatgctgatagagacactcctgtaagcatttaactctcttgtaaatttgtcgttttctttttattgaatttgttatttcacaattgtgttccacaggcacaatgcacttattcaagattatagagaattgtagctttcatcactgcattcgacctatggacatcttgatttactaataatacagtaagaacttatgggacaaattttataagatggttaaccagccagaagattggataattgatgtggtgtagttccatgcaatatacaattaattatttcgtccttcagttgactttccctcttagtttgtaggcgtattcatgaattgttgttccatatgtgtataaatgcacaggaagtgcgtatataccatccacttcaagtaatctaactgagcttaaaatttacagcatcttcggacacaatcctcccgaaagaattatgctgatgcaagccacgtttctgctgtcattttgggtggaggcaagggagttggcctctttcctctgacaagcacaagggctacccccgctgtaagaaagtgaaacactatttatatctaatgttgactactgtactgtgcagataatagatttttaattgtggataacatacttagaacttaagaaaaagtttggaatctcttgtcagtttaaactttaaacaagtgctaattttaacttgctgttcttaatgtctgtcgcttaacttgattgagtgactgcaaatcaagttacaacaacatctggtatcatttggttatttcgacttgcttactatgtcatacggttactacattactatccatttggaactactctttctaggggcctagactaatgagacacactaatcaatatcagagtatcacatcaacacaactaagtattagatacaaggatgctaatcaacaagttatctacttttagaacttcatccgagaaaacatatgctagtagtatgttgtattttaccatgatgcctaaaagcatcaaatttaattctctcataaccttaagccatctaactttcactcatccaatcaggctatcaatgactaggagatagatgattcgaaatttcgttgtcaactgtgaatgaataatctaaattctatttttatttttctattacttaggttcctgttggaggatgttacaggcttattgacatccctatgagcaattgcttcaatagcggaataaataaaatatttgtgatgactcagttcaattctgcttctcttaatcgccatatccatcatacataccttggtggggggatcaactttactgatgggtctgtgcaggtaatttacctccatattcttgatatgccttagtggaaaccagttactagcagtagcaggtagcaacattaattgaactaacgttggcaatatataggtattggctgctacacaaatgcctgacgaaccggctggatggttccagggtacagcagatgctatcagaaaatttatgtggatacttgaggtaattaatttgtttcttccctgatattaatgggtaataattcttgcagaatgtaacttttatcatggatcataaaataccccttctattgcattcctgtccaggatcattacaatcaaaacaacattgagcacgttgtaatcttgtgtggagatcagctttacaggatgaattacatggagcttgtgcaggtgcattatgttgtcattctttttacttcatgtattcttgattttcgattgagcaatactttgacatgtaatacttgcagaaacatgtcgatgacaatgctgacattaccatatcatgtgctcccatcgacgggaggtaaattagttcttgtgcatcctgattctatgagcgtgccttatccaacataagatgcatgtgttctaattttcttcttttttattgggaagccgagcttctgactatggactggtgaagtttgatgattcaggccgtgtgatccaatttcttgagaaaccagagggtgctgatttggaatcgatggttagtaattgtcatctttactgttctcttgagaaatcttttctttagttcatctgcacggttggtctgtcagaaataaatatctatttctgcataatgctgcactaaatcaactgatcactgcagaaagttgataccagcttcctcagctatgcaatagatgacaaacaaaagtatccctacattgcatcaatgggaatttatgtcttgaaaaaggatgttcttttagacattctgaagtaagtgtcacccatttgtgatagttcggtccacttcctatgtcttctgataattcgtattcttaggtcaaaatatgcacacctgcaagactttggatcagaaattctcccgagagctgttttagagcataatgtgaaggtaagttccgaacaactgtaatccgggtttcctacataatctccatctattgcttttgcatattcaatggtttagttaatgctacctccacccacagtggacatcaacacagtctgaaaacacaagtttgaccagtagtttccattgtgatatacttatagagtatgataaatttacagtctgaatatccttagtgttttgtggttgctggagcgctgggagtaactagcatgacaacacccagagcaatatacagatcaatgagctttttacattttgcctactctatataacttgaactagcactatattgttttaggcatgtgtcttcacagaatattgggaggatattggaacaatcaaatcattttttgatgctaacttggccctcaccgagcaggtattgccacatatgtattctgtacaactgatgcactctttaacatattgcgagacaccttgtacagtaatacccaaagatgtcttacagagtctaatgatttacccattattcttcagttaacttactgctaacaccaatttgtatacaaaagttgtctgaacatgtttctccgttccagcctccaaagttcgaattttatgatccaaaaactcctttcttcacctcacctcgatacttacctccagcaagattggagaagtgcaaggtatgagtcatgtaaaccacagttgttgcttcagatcacagcaaatgcaatttaaattttggatcttttttttgtgcagatcaaagatgcaataatctccgatggctgttcctttagtgaatgcaccattgagcattctgtcattggaatctcttcacgcgttagcatcgggtgtgaactcaaggtactttatcaagacataaagctcatagtttggacattcaatgcatcaaaatttgatgtcttattaagattagattgattcataggtactgtttggaaactaccaattattctcatgtagttttgcaattctaaaattcacaggataccatgatgatgggtgcagatcaatatgaaactgaagaagaaacttcaaagctattgtttgaaggcaaggtcccaattggtataggcgagaacacaaagataaggtgagccacatctggactaatgcttacatattactgtccttacctggcaatatcgtgtttccaaatgtatgagaccttcgtttcatgaattttaggaactgtatcatcgacatgaatgctagaattggcaggaacgtgatcattgcaaacactcaggtgagcaagtatatgtatttaactactccaaagtcgatgaactcatcttttataaacacttggtaaccacacgagttcactgcgtatgatagggggtccaagagtctgatcaccctgaagagggatactacataaggtctggaattgtggtgatcctgaagaatgcaaccatcaaggacgggaccgtcatataa

SEQ ID NO: 56 - DNA, 4028 bp, Oryza sativa
  misc_feature (1)..(4028): Modified AGPase large subunit gDNA
  misc_feature (1469)..(1471): G158N mutation
  misc_feature (1484)..(1486): D163G mutation
atgcaattcatgatgccattggatacgaatgcttgtgcacaaccaatgagaagggctggtgagggtgctgggactgagaggttgatggaaagattgaatattgggggcatgactcaggagaaagcactgaggaagaggtgctttggtgatggagttactggaactgcacgatgtgtgttcacatcagatgctgatagagacactcctgtaagcatttaactctcttgtaaatttgtcgttttctttttattgaatttgttatttcacaattgtgttccacaggcacaatgcacttattcaagattatagagaattgtagctttcatcactgcattcgacctatggacatcttgatttactaataatacagtaagaacttatgggacaaattttataagatggttaaccagccagaagattggataattgatgtggtgtagttccatgcaatatacaattaattatttcgtccttcagttgactttccctcttagtttgtaggcgtattcatgaattgttgttccatatgtgtataaatgcacaggaagtgcgtatataccatccacttcaagtaatctaactgagcttaaaatttacagcatcttcggacacaatcctcccgaaagaattatgctgatgcaagccacgtttctgctgtcattttgggtggaggcactggagttcaactctttcctctgacaagcacaagggctacccccgctgtaagaaagtgaaacactatttatatctaatgttgactactgtactgtgcagataatagatttttaattgtggataacatacttagaacttaagaaaaagtttggaatctcttgtcagtttaaactttaaacaagtgctaattttaacttgctgttcttaatgtctgtcgcttaacttgattgagtgactgcaaatcaagttacaacaacatctggtatcatttggttatttcgacttgcttactatgtcatacggttactacattactatccatttggaactactctttctaggggcctagactaatgagacacactaatcaatatcagagtatcacatcaacacaactaagtattagatacaaggatgctaatcaacaagttatctacttttagaacttcatccgagaaaacatatgctagtagtatgttgtattttaccatgatgcctaaaagcatcaaatttaattctctcataaccttaagccatctaactttcactcatccaatcaggctatcaatgactaggagatagatgattcgaaatttcgttgtcaactgtgaatgaataatctaaattctatttttatttttctattacttaggttcctgttggaggatgttacaggcttattgacatccctatgagcaattgcttcaatagcggaataaataaaatatttgtgatgactcagttcaattctgcttctcttaatcgccatatccatcatacataccttggtgggaacatcaactttactggcgggtctgtgcaggtaatttacctccatattcttgatatgccttagtggaaaccagttactagcagtagcaggtagcaacattaattgaactaacgttggcaatatataggtattggctgctacacaaatgcctgacgaaccggctggatggttccagggtacagcagatgctatcagaaaatttatgtggatacttgaggtaattaatttgtttcttccctgatattaatgggtaataattcttgcagaatgtaacttttatcatggatcataaaataccccttctattgcattcctgtccaggatcattacaatcaaaacaacattgagcacgttgtaatcttgtgtggagatcagctttacaggatgaattacatggagcttgtgcaggtgcattatgttgtcattctttttacttcatgtattcttgattttcgattgagcaatactttgacatgtaatacttgcagaaacatgtcgatgacaatgctgacattaccatatcatgtgctcccatcgacgggaggtaaattagttcttgtgcatcctgattctatgagcgtgccttatccaacataagatgcatgtgttctaattttcttcttttttattgggaagccgagcttctgactatggactggtgaagtttgatgattcaggccgtgtgatccaatttcttgagaaaccagagggtgctgatttggaatcgatggttagtaattgtcatctttactgttctcttgagaaatcttttctttagttcatctgcacggttggtctgtcagaaataaatatctatttctgcataatgctgcactaaatcaactgatcactgcagaaagttgataccagcttcctcagctatgcaatagatgacaaacaaaagtatccctacattgcatcaatgggaatttatgtcttgaaaaaggatgttcttttagacattctgaagtaagtgtcacccatttgtgatagttcggtccacttcctatgtcttctgataattcgtattcttaggtcaaaatatgcacacctgcaagactttggatcagaaattctcccgagagctgttttagagcataatgtgaaggtaagttccgaacaactgtaatccgggtttcctacataatctccatctattgcttttgcatattcaatggtttagttaatgctacctccacccacagtggacatcaacacagtctgaaaacacaagtttgaccagtagtttccattgtgatatacttatagagtatgataaatttacagtctgaatatccttagtgttttgtggttgctggagcgctgggagtaactagcatgacaacacccagagcaatatacagatcaatgagctttttacattttgcctactctatataacttgaactagcactatattgttttaggcatgtgtcttcacagaatattgggaggatattggaacaatcaaatcattttttgatgctaacttggccctcaccgagcaggtattgccacatatgtattctgtacaactgatgcactctttaacatattgcgagacaccttgtacagtaatacccaaagatgtcttacagagtctaatgatttacccattattcttcagttaacttactgctaacaccaatttgtatacaaaagttgtctgaacatgtttctccgttccagcctccaaagttcgaattttatgatccaaaaactcctttcttcacctcacctcgatacttacctccagcaagattggagaagtgcaaggtatgagtcatgtaaaccacagttgttgcttcagatcacagcaaatgcaatttaaattttggatcttttttttgtgcagatcaaagatgcaataatctccgatggctgttcctttagtgaatgcaccattgagcattctgtcattggaatctcttcacgcgttagcatcgggtgtgaactcaaggtactttatcaagacataaagctcatagtttggacattcaatgcatcaaaatttgatgtcttattaagattagattgattcataggtactgtttggaaactaccaattattctcatgtagttttgcaattctaaaattcacaggataccatgatgatgggtgcagatcaatatgaaactgaagaagaaacttcaaagctattgtttgaaggcaaggtcccaattggtataggcgagaacacaaagataaggtgagccacatctggactaatgcttacatattactgtccttacctggcaatatcgtgtttccaaatgtatgagaccttcgtttcatgaattttaggaactgtatcatcgacatgaatgctagaattggcaggaacgtgatcattgcaaacactcaggtgagcaagtatatgtatttaactactccaaagtcgatgaactcatcttttataaacacttggtaaccacacgagttcactgcgtatgatagggggtccaagagtctgatcaccctgaagagggatactacataaggtctggaattgtggtgatcctgaagaatgcaaccatcaaggacgggaccgtcatataa

SEQ ID NO: 57 - DNA, 4028 bp, Oryza sativa
  misc_feature (1)..(4028): Modified AGPase large subunit gDNA
  misc_feature (675)..(677): T95K mutation
  misc_feature (684)..(686): Q98G mutation
  misc_feature (1469)..(1471): G158N mutation
  misc_feature (1484)..(1486): D163G mutation
atgcaattcatgatgccattggatacgaatgcttgtgcacaaccaatgagaagggctggtgagggtgctgggactgagaggttgatggaaagattgaatattgggggcatgactcaggagaaagcactgaggaagaggtgctttggtgatggagttactggaactgcacgatgtgtgttcacatcagatgctgatagagacactcctgtaagcatttaactctcttgtaaatttgtcgttttctttttattgaatttgttatttcacaattgtgttccacaggcacaatgcacttattcaagattatagagaattgtagctttcatcactgcattcgacctatggacatcttgatttactaataatacagtaagaacttatgggacaaattttataagatggttaaccagccagaagattggataattgatgtggtgtagttccatgcaatatacaattaattatttcgtccttcagttgactttccctcttagtttgtaggcgtattcatgaattgttgttccatatgtgtataaatgcacaggaagtgcgtatataccatccacttcaagtaatctaactgagcttaaaatttacagcatcttcggacacaatcctcccgaaagaattatgctgatgcaagccacgtttctgctgtcattttgggtggaggcaagggagttggcctctttcctctgacaagcacaagggctacccccgctgtaagaaagtgaaacactatttatatctaatgttgactactgtactgtgcagataatagatttttaattgtggataacatacttagaacttaagaaaaagtttggaatctcttgtcagtttaaactttaaacaagtgctaattttaacttgctgttcttaatgtctgtcgcttaacttgattgagtgactgcaaatcaagttacaacaacatctggtatcatttggttatttcgacttgcttactatgtcatacggttactacattactatccatttggaactactctttctaggggcctagactaatgagacacactaatcaatatcagagtatcacatcaacacaactaagtattagatacaaggatgctaatcaacaagttatctacttttagaacttcatccgagaaaacatatgctagtagtatgttgtattttaccatgatgcctaaaagcatcaaatttaattctctcataaccttaagccatctaactttcactcatccaatcaggctatcaatgactaggagatagatgattcgaaatttcgttgtcaactgtgaatgaataatctaaattctatttttatttttctattacttaggttcctgttggaggatgttacaggcttattgacatccctatgagcaattgcttcaatagcggaataaataaaatatttgtgatgactcagttcaattctgcttctcttaatcgccatatccatcatacataccttggtgggaacatcaactttactggcgggtctgtgcaggtaatttacctccatattcttgatatgccttagtggaaaccagttactagcagtagcaggtagcaacattaattgaactaacgttggcaatatataggtattggctgctacacaaatgcctgacgaaccggctggatggttccagggtacagcagatgctatcagaaaatttatgtggatacttgaggtaattaatttgtttcttccctgatattaatgggtaataattcttgcagaatgtaacttttatcatggatcataaaataccccttctattgcattcctgtccaggatcattacaatcaaaacaacattgagcacgttgtaatcttgtgtggagatcagctttacaggatgaattacatggagcttgtgcaggtgcattatgttgtcattctttttacttcatgtattcttgattttcgattgagcaatactttgacatgtaatacttgcagaaacatgtcgatgacaatgctgacattaccatatcatgtgctcccatcgacgggaggtaaattagttcttgtgcatcctgattctatgagcgtgccttatccaacataagatgcatgtgttctaattttcttcttttttattgggaagccgagcttctgactatggactggtgaagtttgatgattcaggccgtgtgatccaatttcttgagaaaccagagggtgctgatttggaatcgatggttagtaattgtcatctttactgttctcttgagaaatcttttctttagttcatctgcacggttggtctgtcagaaataaatatctatttctgcataatgctgcactaaatcaactgatcactgcagaaagttgataccagcttcctcagctatgcaatagatgacaaacaaaagtatccctacattgcatcaatgggaatttatgtcttgaaaaaggatgttcttttagacattctgaagtaagtgtcacccatttgtgatagttcggtccacttcctatgtcttctgataattcgtattcttaggtcaaaatatgcacacctgcaagactttggatcagaaattctcccgagagctgttttagagcataatgtgaaggtaagttccgaacaactgtaatccgggtttcctacataatctccatctattgcttttgcatattcaatggtttagttaatgctacctccacccacagtggacatcaacacagtctgaaaacacaagtttgaccagtagtttccattgtgatatacttatagagtatgataaatttacagtctgaatatccttagtgttttgtggttgctggagcgctgggagtaactagcatgacaacacccagagcaatatacagatcaatgagctttttacattttgcctactctatataacttgaactagcactatattgttttaggcatgtgtcttcacagaatattgggaggatattggaacaatcaaatcattttttgatgctaacttggccctcaccgagcaggtattgccacatatgtattctgtacaactgatgcactctttaacatattgcgagacaccttgtacagtaatacccaaagatgtcttacagagtctaatgatttacccattattcttcagttaacttactgctaacaccaatttgtatacaaaagttgtctgaacatgtttctccgttccagcctccaaagttcgaattttatgatccaaaaactcctttcttcacctcacctcgatacttacctccagcaagattggagaagtgcaaggtatgagtcatgtaaaccacagttgttgcttcagatcacagcaaatgcaatttaaattttggatcttttttttgtgcagatcaaagatgcaataatctccgatggctgttcctttagtgaatgcaccattgagcattctgtcattggaatctcttcacgcgttagcatcgggtgtgaactcaaggtactttatcaagacataaagctcatagtttggacattcaatgcatcaaaatttgatgtcttattaagattagattgattcataggtactgtttggaaactaccaattattctcatgtagttttgcaattctaaaattcacaggataccatgatgatgggtgcagatcaatatgaaactgaagaagaaacttcaaagctattgtttgaaggcaaggtcccaattggtataggcgagaacacaaagataaggtgagccacatctggactaatgcttacatattactgtccttacctggcaatatcgtgtttccaaatgtatgagaccttcgtttcatgaattttaggaactgtatcatcgacatgaatgctagaattggcaggaacgtgatcattgcaaacactcaggtgagcaagtatatgtatttaactactccaaagtcgatgaactcatcttttataaacacttggtaaccacacgagttcactgcgtatgatagggggtccaagagtctgatcaccctgaagagggatactacataaggtctggaattgtggtgatcctgaagaatgcaaccatcaaggacgggaccgtcatataa

SEQ ID NO: 58 - DNA, 4028 bp, Oryza sativa
  misc_feature (1)..(4028): Modified AGPase large subunit gDNA
  misc_feature (675)..(677): T95K mutation
  misc_feature (684)..(686): Q98G mutation
  misc_feature (1469)..(1471): G158N mutation
  misc_feature (1484)..(1486): D163G mutation
  misc_feature (3617)..(3619): T445R mutation
atgcaattcatgatgccattggatacgaatgcttgtgcacaaccaatgagaagggctggtgagggtgctgggactgagaggttgatggaaagattgaatattgggggcatgactcaggagaaagcactgaggaagaggtgctttggtgatggagttactggaactgcacgatgtgtgttcacatcagatgctgatagagacactcctgtaagcatttaactctcttgtaaatttgtcgttttctttttattgaatttgttatttcacaattgtgttccacaggcacaatgcacttattcaagattatagagaattgtagctttcatcactgcattcgacctatggacatcttgatttactaataatacagtaagaacttatgggacaaattttataagatggttaaccagccagaagattggataattgatgtggtgtagttccatgcaatatacaattaattatttcgtccttcagttgactttccctcttagtttgtaggcgtattcatgaattgttgttccatatgtgtataaatgcacaggaagtgcgtatataccatccacttcaagtaatctaactgagcttaaaatttacagcatcttcggacacaatcctcccgaaagaattatgctgatgcaagccacgtttctgctgtcattttgggtggaggcaagggagttggcctctttcctctgacaagcacaagggctacccccgctgtaagaaagtgaaacactatttatatctaatgttgactactgtactgtgcagataatagatttttaattgtggataacatacttagaacttaagaaaaagtttggaatctcttgtcagtttaaactttaaacaagtgctaattttaacttgctgttcttaatgtctgtcgcttaacttgattgagtgactgcaaatcaagttacaacaacatctggtatcatttggttatttcgacttgcttactatgtcatacggttactacattactatccatttggaactactctttctaggggcctagactaatgagacacactaatcaatatcagagtatcacatcaacacaactaagtattagatacaaggatgctaatcaacaagttatctacttttagaacttcatccgagaaaacatatgctagtagtatgttgtattttaccatgatgcctaaaagcatcaaatttaattctctcataaccttaagccatctaactttcactcatccaatcaggctatcaatgactaggagatagatgattcgaaatttcgttgtcaactgtgaatgaataatctaaattctatttttatttttctattacttaggttcctgttggaggatgttacaggcttattgacatccctatgagcaattgcttcaatagcggaataaataaaatatttgtgatgactcagttcaattctgcttctcttaatcgccatatccatcatacataccttggtgggaacatcaactttactggcgggtctgtgcaggtaatttacctccatattcttgatatgccttagtggaaaccagttactagcagtagcaggtagcaacattaattgaactaacgttggcaatatataggtattggctgctacacaaatgcctgacgaaccggctggatggttccagggtacagcagatgctatcagaaaatttatgtggatacttgaggtaattaatttgtttcttccctgatattaatgggtaataattcttgcagaatgtaacttttatcatggatcataaaataccccttctattgcattcctgtccaggatcattacaatcaaaacaacattgagcacgttgtaatcttgtgtggagatcagctttacaggatgaattacatggagcttgtgcaggtgcattatgttgtcattctttttacttcatgtattcttgattttcgattgagcaatactttgacatgtaatacttgcagaaacatgtcgatgacaatgctgacattaccatatcatgtgctcccatcgacgggaggtaaattagttcttgtgcatcctgattctatgagcgtgccttatccaacataagatgcatgtgttctaattttcttcttttttattgggaagccgagcttctgactatggactggtgaagtttgatgattcaggccgtgtgatccaatttcttgagaaaccagagggtgctgatttggaatcgatggttagtaattgtcatctttactgttctcttgagaaatcttttctttagttcatctgcacggttggtctgtcagaaataaatatctatttctgcataatgctgcactaaatcaactgatcactgcagaaagttgataccagcttcctcagctatgcaatagatgacaaacaaaagtatccctacattgcatcaatgggaatttatgtcttgaaaaaggatgttcttttagacattctgaagtaagtgtcacccatttgtgatagttcggtccacttcctatgtcttctgataattcgtattcttaggtcaaaatatgcacacctgcaagactttggatcagaaattctcccgagagctgttttagagcataatgtgaaggtaagttccgaacaactgtaatccgggtttcctacataatctccatctattgcttttgcatattcaatggtttagttaatgctacctccacccacagtggacatcaacacagtctgaaaacacaagtttgaccagtagtttccattgtgatatacttatagagtatgataaatttacagtctgaatatccttagtgttttgtggttgctggagcgctgggagtaactagcatgacaacacccagagcaatatacagatcaatgagctttttacattttgcctactctatataacttgaactagcactatattgttttaggcatgtgtcttcacagaatattgggaggatattggaacaatcaaatcattttttgatgctaacttggccctcaccgagcaggtattgccacatatgtattctgtacaactgatgcactctttaacatattgcgagacaccttgtacagtaatacccaaagatgtcttacagagtctaatgatttacccattattcttcagttaacttactgctaacaccaatttgtatacaaaagttgtctgaacatgtttctccgttccagcctccaaagttcgaattttatgatccaaaaactcctttcttcacctcacctcgatacttacctccagcaagattggagaagtgcaaggtatgagtcatgtaaaccacagttgttgcttcagatcacagcaaatgcaatttaaattttggatcttttttttgtgcagatcaaagatgcaataatctccgatggctgttcctttagtgaatgcaccattgagcattctgtcattggaatctcttcacgcgttagcatcgggtgtgaactcaaggtactttatcaagacataaagctcatagtttggacattcaatgcatcaaaatttgatgtcttattaagattagattgattcataggtactgtttggaaactaccaattattctcatgtagttttgcaattctaaaattcacaggataccatgatgatgggtgcagatcaatatgaaactgaagaagaaaggtcaaagctattgtttgaaggcaaggtcccaattggtataggcgagaacacaaagataaggtgagccacatctggactaatgcttacatattactgtccttacctggcaatatcgtgtttccaaatgtatgagaccttcgtttcatgaattttaggaactgtatcatcgacatgaatgctagaattggcaggaacgtgatcattgcaaacactcaggtgagcaagtatatgtatttaactactccaaagtcgatgaactcatcttttataaacacttggtaaccacacgagttcactgcgtatgatagggggtccaagagtctgatcaccctgaagagggatactacataaggtctggaattgtggtgatcctgaagaatgcaaccatcaaggacgggaccgtcatataa

SEQ ID NO: 59 - DNA, 4028 bp, Oryza sativa
  misc_feature (1)..(4028): Modified AGPase large subunit gDNA
  misc_feature (675)..(677): T95K mutation
  misc_feature (684)..(686): Q98G mutation
  misc_feature (3617)..(3619): T445R mutation
atgcaattcatgatgccattggatacgaatgcttgtgcacaaccaatgagaagggctggtgagggtgctgggactgagaggttgatggaaagattgaatattgggggcatgactcaggagaaagcactgaggaagaggtgctttggtgatggagttactggaactgcacgatgtgtgttcacatcagatgctgatagagacactcctgtaagcatttaactctcttgtaaatttgtcgttttctttttattgaatttgttatttcacaattgtgttccacaggcacaatgcacttattcaagattatagagaattgtagctttcatcactgcattcgacctatggacatcttgatttactaataatacagtaagaacttatgggacaaattttataagatggttaaccagccagaagattggataattgatgtggtgtagttccatgcaatatacaattaattatttcgtccttcagttgactttccctcttagtttgtaggcgtattcatgaattgttgttccatatgtgtataaatgcacaggaagtgcgtatataccatccacttcaagtaatctaactgagcttaaaatttacagcatcttcggacacaatcctcccgaaagaattatgctgatgcaagccacgtttctgctgtcattttgggtggaggcaagggagttggcctctttcctctgacaagcacaagggctacccccgctgtaagaaagtgaaacactatttatatctaatgttgactactgtactgtgcagataatagatttttaattgtggataacatacttagaacttaagaaaaagtttggaatctcttgtcagtttaaactttaaacaagtgctaattttaacttgctgttcttaatgtctgtcgcttaacttgattgagtgactgcaaatcaagttacaacaacatctggtatcatttggttatttcgacttgcttactatgtcatacggttactacattactatccatttggaactactctttctaggggcctagactaatgagacacactaatcaatatcagagtatcacatcaacacaactaagtattagatacaaggatgctaatcaacaagttatctacttttagaacttcatccgagaaaacatatgctagtagtatgttgtattttaccatgatgcctaaaagcatcaaatttaattctctcataaccttaagccatctaactttcactcatccaatcaggctatcaatgactaggagatagatgattcgaaatttcgttgtcaactgtgaatgaataatctaaattctatttttatttttctattacttaggttcctgttggaggatgttacaggcttattgacatccctatgagcaattgcttcaatagcggaataaataaaatatttgtgatgactcagttcaattctgcttctcttaatcgccatatccatcatacataccttggtggggggatcaactttactgatgggtctgtgcaggtaatttacctccatattcttgatatgccttagtggaaaccagttactagcagtagcaggtagcaacattaattgaactaacgttggcaatatataggtattggctgctacacaaatgcctgacgaaccggctggatggttccagggtacagcagatgctatcagaaaatttatgtggatacttgaggtaattaatttgtttcttccctgatattaatgggtaataattcttgcagaatgtaacttttatcatggatcataaaataccccttctattgcattcctgtccaggatcattacaatcaaaacaacattgagcacgttgtaatcttgtgtggagatcagctttacaggatgaattacatggagcttgtgcaggtgcattatgttgtcattctttttacttcatgtattcttgattttcgattgagcaatactttgacatgtaatacttgcagaaacatgtcgatgacaatgctgacattaccatatcatgtgctcccatcgacgggaggtaaattagttcttgtgcatcctgattctatgagcgtgccttatccaacataagatgcatgtgttctaattttcttcttttttattgggaagccgagcttctgactatggactggtgaagtttgatgattcaggccgtgtgatccaatttcttgagaaaccagagggtgctgatttggaatcgatggttagtaattgtcatctttactgttctcttgagaaatcttttctttagttcatctgcacggttggtctgtcagaaataaatatctatttctgcataatgctgcactaaatcaactgatcactgcagaaagttgataccagcttcctcagctatgcaatagatgacaaacaaaagtatccctacattgcatcaatgggaatttatgtcttgaaaaaggatgttcttttagacattctgaagtaagtgtcacccatttgtgatagttcggtccacttcctatgtcttctgataattcgtattcttaggtcaaaatatgcacacctgcaagactttggatcagaaattctcccgagagctgttttagagcataatgtgaaggtaagttccgaacaactgtaatccgggtttcctacataatctccatctattgcttttgcatattcaatggtttagttaatgctacctccacccacagtggacatcaacacagtctgaaaacacaagtttgaccagtagtttccattgtgatatacttatagagtatgataaatttacagtctgaatatccttagtgttttgtggttgctggagcgctgggagtaactagcatgacaacacccagagcaatatacagatcaatgagctttttacattttgcctactctatataacttgaactagcactatattgttttaggcatgtgtcttcacagaatattgggaggatattggaacaatcaaatcattttttgatgctaacttggccctcaccgagcaggtattgccacatatgtattctgtacaactgatgcactctttaacatattgcgagacaccttgtacagtaatacccaaagatgtcttacagagtctaatgatttacccattattcttcagttaacttactgctaacaccaatttgtatacaaaagttgtctgaacatgtttctccgttccagcctccaaagttcgaattttatgatccaaaaactcctttcttcacctcacctcgatacttacctccagcaagattggagaagtgcaaggtatgagtcatgtaaaccacagttgttgcttcagatcacagcaaatgcaatttaaattttggatcttttttttgtgcagatcaaagatgcaataatctccgatggctgttcctttagtgaatgcaccattgagcattctgtcattggaatctcttcacgcgttagcatcgggtgtgaactcaaggtactttatcaagacataaagctcatagtttggacattcaatgcatcaaaatttgatgtcttattaagattagattgattcataggtactgtttggaaactaccaattattctcatgtagttttgcaattctaaaattcacaggataccatgatgatgggtgcagatcaatatgaaactgaagaagaaaggtcaaagctattgtttgaaggcaaggtcccaattggtataggcgagaacacaaagataaggtgagccacatctggactaatgcttacatattactgtccttacctggcaatatcgtgtttccaaatgtatgagaccttcgtttcatgaattttaggaactgtatcatcgacatgaatgctagaattggcaggaacgtgatcattgcaaacactcaggtgagcaagtatatgtatttaactactccaaagtcgatgaactcatcttttataaacacttggtaaccacacgagttcactgcgtatgatagggggtccaagagtctgatcaccctgaagagggatactacataaggtctggaattgtggtgatcctgaagaatgcaaccatcaaggacgggaccgtcatataa

SEQ ID NO: 60 - DNA, 4028 bp, Oryza sativa
  misc_feature (1)..(4028): Modified AGPase large subunit gDNA
  misc_feature (1469)..(1471): G158N mutation
  misc_feature (1484)..(1486): D163G mutation
  misc_feature (3617)..(3619): T445R mutation
atgcaattcatgatgccattggatacgaatgcttgtgcacaaccaatgagaagggctggtgagggtgctgggactgagaggttgatggaaagattgaatattgggggcatgactcaggagaaagcactgaggaagaggtgctttggtgatggagttactggaactgcacgatgtgtgttcacatcagatgctgatagagacactcctgtaagcatttaactctcttgtaaatttgtcgttttctttttattgaatttgttatttcacaattgtgttccacaggcacaatgcacttattcaagattatagagaattgtagctttcatcactgcattcgacctatggacatcttgatttactaataatacagtaagaacttatgggacaaattttataagatggttaaccagccagaagattggataattgatgtggtgtagttccatgcaatatacaattaattatttcgtccttcagttgactttccctcttagtttgtaggcgtattcatgaattgttgttccatatgtgtataaatgcacaggaagtgcgtatataccatccacttcaagtaatctaactgagcttaaaatttacagcatcttcggacacaatcctcccgaaagaattatgctgatgcaagccacgtttctgctgtcattttgggtggaggcactggagttcaactctttcctctgacaagcacaagggctacccccgctgtaagaaagtgaaacactatttatatctaatgttgactactgtactgtgcagataatagatttttaattgtggataacatacttagaacttaagaaaaagtttggaatctcttgtcagtttaaactttaaacaagtgctaattttaacttgctgttcttaatgtctgtcgcttaacttgattgagtgactgcaaatcaagttacaacaacatctggtatcatttggttatttcgacttgcttactatgtcatacggttactacattactatccatttggaactactctttctaggggcctagactaatgagacacactaatcaatatcagagtatcacatcaacacaactaagtattagatacaaggatgctaatcaacaagttatctacttttagaacttcatccgagaaaacatatgctagtagtatgttgtattttaccatgatgcctaaaagcatcaaatttaattctctcataaccttaagccatctaactttcactcatccaatcaggctatcaatgactaggagatagatgattcgaaatttcgttgtcaactgtgaatgaataatctaaattctatttttatttttctattacttaggttcctgttggaggatgttacaggcttattgacatccctatgagcaattgcttcaatagcggaataaataaaatatttgtgatgactcagttcaattctgcttctcttaatcgccatatccatcatacataccttggtgggaacatcaactttactggcgggtctgtgcaggtaatttacctccatattcttgatatgccttagtggaaaccagttactagcagtagcaggtagcaacattaattgaactaacgttggcaatatataggtattggctgctacacaaatgcctgacgaaccggctggatggttccagggtacagcagatgctatcagaaaatttatgtggatacttgaggtaattaatttgtttcttccctgatattaatgggtaataattcttgcagaatgtaacttttatcatggatcataaaataccccttctattgcattcctgtccaggatcattacaatcaaaacaacattgagcacgttgtaatcttgtgtggagatcagctttacaggatgaattacatggagcttgtgcaggtgcattatgttgtcattctttttacttcatgtattcttgattttcgattgagcaatactttgacatgtaatacttgcagaaacatgtcgatgacaatgctgacattaccatatcatgtgctcccatcgacgggaggtaaattagttcttgtgcatcctgattctatgagcgtgccttatccaacataagatgcatgtgttctaattttcttcttttttattgggaagccgagcttctgactatggactggtgaagtttgatgattcaggccgtgtgatccaatttcttgagaaaccagagggtgctgatttggaatcgatggttagtaattgtcatctttactgttctcttgagaaatcttttctttagttcatctgcacggttggtctgtcagaaataaatatctatttctgcataatgctgcactaaatcaactgatcactgcagaaagttgataccagcttcctcagctatgcaatagatgacaaacaaaagtatccctacattgcatcaatgggaatttatgtcttgaaaaaggatgttcttttagacattctgaagtaagtgtcacccatttgtgatagttcggtccacttcctatgtcttctgataattcgtattcttaggtcaaaatatgcacacctgcaagactttggatcagaaattctcccgagagctgttttagagcataatgtgaaggtaagttccgaacaactgtaatccgggtttcctacataatctccatctattgcttttgcatattcaatggtttagttaatgctacctccacccacagtggacatcaacacagtctgaaaacacaagtttgaccagtagtttccattgtgatatacttatagagtatgataaatttacagtctgaatatccttagtgttttgtggttgctggagcgctgggagtaactagcatgacaacacccagagcaatatacagatcaatgagctttttacattttgcctactctatataacttgaactagcactatattgttttaggcatgtgtcttcacagaatattgggaggatattggaacaatcaaatcattttttgatgctaacttggccctcaccgagcaggtattgccacatatgtattctgtacaactgatgcactctttaacatattgcgagacaccttgtacagtaatacccaaagatgtcttacagagtctaatgatttacccattattcttcagttaacttactgctaacaccaatttgtatacaaaagttgtctgaacatgtttctccgttccagcctccaaagttcgaattttatgatccaaaaactcctttcttcacctcacctcgatacttacctccagcaagattggagaagtgcaaggtatgagtcatgtaaaccacagttgttgcttcagatcacagcaaatgcaatttaaattttggatcttttttttgtgcagatcaaagatgcaataatctccgatggctgttcctttagtgaatgcaccattgagcattctgtcattggaatctcttcacgcgttagcatcgggtgtgaactcaaggtactttatcaagacataaagctcatagtttggacattcaatgcatcaaaatttgatgtcttattaagattagattgattcataggtactgtttggaaactaccaattattctcatgtagttttgcaattctaaaattcacaggataccatgatgatgggtgcagatcaatatgaaactgaagaagaaaggtcaaagctattgtttgaaggcaaggtcccaattggtataggcgagaacacaaagataaggtgagccacatctggactaatgcttacatattactgtccttacctggcaatatcgtgtttccaaatgtatgagaccttcgtttcatgaattttaggaactgtatcatcgacatgaatgctagaattggcaggaacgtgatcattgcaaacactcaggtgagcaagtatatgtatttaactactccaaagtcgatgaactcatcttttataaacacttggtaaccacacgagttcactgcgtatgatagggggtccaagagtctgatcaccctgaagagggatactacataaggtctggaattgtggtgatcctgaagaatgcaaccatcaaggacgggaccgtcatataa

SEQ ID NO: 61 - DNA, 1557 bp, Oryza sativa
  misc_feature (1)..(1557): Modified AGPase large subunit cDNA
  misc_feature (283)..(285): T95K mutation
  misc_feature (292)..(294): Q98G mutation
atgcaattcatgatgccattggatacgaatgcttgtgcacaaccaatgagaagggctggtgagggtgctgggactgagaggttgatggaaagattgaatattgggggcatgactcaggagaaagcactgaggaagaggtgctttggtgatggagttactggaactgcacgatgtgtgttcacatcagatgctgatagagacactcctcatcttcggacacaatcctcccgaaagaattatgctgatgcaagccacgtttctgctgtcattttgggtggaggcaagggagttggcctctttcctctgacaagcacaagggctacccccgctgttcctgttggaggatgttacaggcttattgacatccctatgagcaattgcttcaatagcggaataaataaaatatttgtgatgactcagttcaattctgcttctcttaatcgccatatccatcatacataccttggtggggggatcaactttactgatgggtctgtgcaggtattggctgctacacaaatgcctgacgaaccggctggatggttccagggtacagcagatgctatcagaaaatttatgtggatacttgaggatcattacaatcaaaacaacattgagcacgttgtaatcttgtgtggagatcagctttacaggatgaattacatggagcttgtgcagaaacatgtcgatgacaatgctgacattaccatatcatgtgctcccatcgacgggagccgagcttctgactatggactggtgaagtttgatgattcaggccgtgtgatccaatttcttgagaaaccagagggtgctgatttggaatcgatgaaagttgataccagcttcctcagctatgcaatagatgacaaacaaaagtatccctacattgcatcaatgggaatttatgtcttgaaaaaggatgttcttttagacattctgaagtcaaaatatgcacacctgcaagactttggatcagaaattctcccgagagctgttttagagcataatgtgaaggcatgtgtcttcacagaatattgggaggatattggaacaatcaaatcattttttgatgctaacttggccctcaccgagcagcctccaaagttcgaattttatgatccaaaaactcctttcttcacctcacctcgatacttacctccagcaagattggagaagtgcaagatcaaagatgcaataatctccgatggctgttcctttagtgaatgcaccattgagcattctgtcattggaatctcttcacgcgttagcatcgggtgtgaactcaaggataccatgatgatgggtgcagatcaatatgaaactgaagaagaaacttcaaagctattgtttgaaggcaaggtcccaattggtataggcgagaacacaaagataaggaactgtatcatcgacatgaatgctagaattggcaggaacgtgatcattgcaaacactcagggggtccaagagtctgatcaccctgaagagggatactacataaggtctggaattgtggtgatcctgaagaatgcaaccatcaaggacgggaccgtcatataa

SEQ ID NO: 62 - DNA, 1557 bp, Oryza sativa
  misc_feature (1)..(1557): Modified AGPase large subunit cDNA
  misc_feature (472)..(474): G158N mutation
  misc_feature (487)..(487): D163G mutation
atgcaattcatgatgccattggatacgaatgcttgtgcacaaccaatgagaagggctggtgagggtgctgggactgagaggttgatggaaagattgaatattgggggcatgactcaggagaaagcactgaggaagaggtgctttggtgatggagttactggaactgcacgatgtgtgttcacatcagatgctgatagagacactcctcatcttcggacacaatcctcccgaaagaattatgctgatgcaagccacgtttctgctgtcattttgggtggaggcactggagttcaactctttcctctgacaagcacaagggctacccccgctgttcctgttggaggatgttacaggcttattgacatccctatgagcaattgcttcaatagcggaataaataaaatatttgtgatgactcagttcaattctgcttctcttaatcgccatatccatcatacataccttggtgggaacatcaactttactggcgggtctgtgcaggtattggctgctacacaaatgcctgacgaaccggctggatggttccagggtacagcagatgctatcagaaaatttatgtggatacttgaggatcattacaatcaaaacaacattgagcacgttgtaatcttgtgtggagatcagctttacaggatgaattacatggagcttgtgcagaaacatgtcgatgacaatgctgacattaccatatcatgtgctcccatcgacgggagccgagcttctgactatggactggtgaagtttgatgattcaggccgtgtgatccaatttcttgagaaaccagagggtgctgatttggaatcgatgaaagttgataccagcttcctcagctatgcaatagatgacaaacaaaagtatccctacattgcatcaatgggaatttatgtcttgaaaaaggatgttcttttagacattctgaagtcaaaatatgcacacctgcaagactttggatcagaaattctcccgagagctgttttagagcataatgtgaaggcatgtgtcttcacagaatattgggaggatattggaacaatcaaatcattttttgatgctaacttggccctcaccgagcagcctccaaagttcgaattttatgatccaaaaactcctttcttcacctcacctcgatacttacctccagcaagattggagaagtgcaagatcaaagatgcaataatctccgatggctgttcctttagtgaatgcaccattgagcattctgtcattggaatctcttcacgcgttagcatcgggtgtgaactcaaggataccatgatgatgggtgcagatcaatatgaaactgaagaagaaacttcaaagctattgtttgaaggcaaggtcccaattggtataggcgagaacacaaagataaggaactgtatcatcgacatgaatgctagaattggcaggaacgtgatcattgcaaacactcagggggtccaagagtctgatcaccctgaagagggatactacataaggtctggaattgtggtgatcctgaagaatgcaaccatcaaggacgggaccgtcatataa

SEQ ID NO: 63 - DNA, 1557 bp, Oryza sativa
  misc_feature (1)..(1557): Modified AGPase large subunit cDNA
  misc_feature (283)..(285): T95K mutation
  misc_feature (292)..(294): Q98G mutation
  misc_feature (472)..(474): G158N mutation
  misc_feature (487)..(489): D163G mutation
atgcaattcatgatgccattggatacgaatgcttgtgcacaaccaatgagaagggctggtgagggtgctgggactgagaggttgatggaaagattgaatattgggggcatgactcaggagaaagcactgaggaagaggtgctttggtgatggagttactggaactgcacgatgtgtgttcacatcagatgctgatagagacactcctcatcttcggacacaatcctcccgaaagaattatgctgatgcaagccacgtttctgctgtcattttgggtggaggcaagggagttggcctctttcctctgacaagcacaagggctacccccgctgttcctgttggaggatgttacaggcttattgacatccctatgagcaattgcttcaatagcggaataaataaaatatttgtgatgactcagttcaattctgcttctcttaatcgccatatccatcatacataccttggtgggaacatcaactttactggcgggtctgtgcaggtattggctgctacacaaatgcctgacgaaccggctggatggttccagggtacagcagatgctatcagaaaatttatgtggatacttgaggatcattacaatcaaaacaacattgagcacgttgtaatcttgtgtggagatcagctttacaggatgaattacatggagcttgtgcagaaacatgtcgatgacaatgctgacattaccatatcatgtgctcccatcgacgggagccgagcttctgactatggactggtgaagtttgatgattcaggccgtgtgatccaatttcttgagaaaccagagggtgctgatttggaatcgatgaaagttgataccagcttcctcagctatgcaatagatgacaaacaaaagtatccctacattgcatcaatgggaatttatgtcttgaaaaaggatgttcttttagacattctgaagtcaaaatatgcacacctgcaagactttggatcagaaattctcccgagagctgttttagagcataatgtgaaggcatgtgtcttcacagaatattgggaggatattggaacaatcaaatcattttttgatgctaacttggccctcaccgagcagcctccaaagttcgaattttatgatccaaaaactcctttcttcacctcacctcgatacttacctccagcaagattggagaagtgcaagatcaaagatgcaataatctccgatggctgttcctttagtgaatgcaccattgagcattctgtcattggaatctcttcacgcgttagcatcgggtgtgaactcaaggataccatgatgatgggtgcagatcaatatgaaactgaagaagaaacttcaaagctattgtttgaaggcaaggtcccaattggtataggcgagaacacaaagataaggaactgtatcatcgacatgaatgctagaattggcaggaacgtgatcattgcaaacactcagggggtccaagagtctgatcaccctgaagagggatactacataaggtctggaattgtggtgatcctgaagaatgcaaccatcaaggacgggaccgtcatataa

SEQ ID NO: 64 - DNA, 1557 bp, Oryza sativa
  misc_feature (1)..(1557): Modified AGPase large subunit cDNA
  misc_feature (283)..(285): T95K mutation
  misc_feature (292)..(294): Q98G mutation
  misc_feature (472)..(474): G158N mutation
  misc_feature (487)..(489): D163G mutation
  misc_feature (1333)..(1335): T445R mutation
atgcaattcatgatgccattggatacgaatgcttgtgcacaaccaatgagaagggctggtgagggtgctgggactgagaggttgatggaaagattgaatattgggggcatgactcaggagaaagcactgaggaagaggtgctttggtgatggagttactggaactgcacgatgtgtgttcacatcagatgctgatagagacactcctcatcttcggacacaatcctcccgaaagaattatgctgatgcaagccacgtttctgctgtcattttgggtggaggcaagggagttggcctctttcctctgacaagcacaagggctacccccgctgttcctgttggaggatgttacaggcttattgacatccctatgagcaattgcttcaatagcggaataaataaaatatttgtgatgactcagttcaattctgcttctcttaatcgccatatccatcatacataccttggtgggaacatcaactttactggcgggtctgtgcaggtattggctgctacacaaatgcctgacgaaccggctggatggttccagggtacagcagatgctatcagaaaatttatgtggatacttgaggatcattacaatcaaaacaacattgagcacgttgtaatcttgtgtggagatcagctttacaggatgaattacatggagcttgtgcagaaacatgtcgatgacaatgctgacattaccatatcatgtgctcccatcgacgggagccgagcttctgactatggactggtgaagtttgatgattcaggccgtgtgatccaatttcttgagaaaccagagggtgctgatttggaatcgatgaaagttgataccagcttcctcagctatgcaatagatgacaaacaaaagtatccctacattgcatcaatgggaatttatgtcttgaaaaaggatgttcttttagacattctgaagtcaaaatatgcacacctgcaagactttggatcagaaattctcccgagagctgttttagagcataatgtgaaggcatgtgtcttcacagaatattgggaggatattggaacaatcaaatcattttttgatgctaacttggccctcaccgagcagcctccaaagttcgaattttatgatccaaaaactcctttcttcacctcacctcgatacttacctccagcaagattggagaagtgcaagatcaaagatgcaataatctccgatggctgttcctttagtgaatgcaccattgagcattctgtcattggaatctcttcacgcgttagcatcgggtgtgaactcaaggataccatgatgatgggtgcagatcaatatgaaactgaagaagaaaggtcaaagctattgtttgaaggcaaggtcccaattggtataggcgagaacacaaagataaggaactgtatcatcgacatgaatgctagaattggcaggaacgtgatcattgcaaacactcagggggtccaagagtctgatcaccctgaagagggatactacataaggtctggaattgtggtgatcctgaagaatgcaaccatcaaggacgggaccgtcatataa

SEQ ID NO: 65 - DNA, 1557 bp, Oryza sativa
  misc_feature (1)..(1557): Modified AGPase large subunit cDNA
  misc_feature (283)..(285): T95K mutation
  misc_feature (292)..(294): Q98G mutation
  misc_feature (1333)..(1335): T445R mutation
atgcaattcatgatgccattggatacgaatgcttgtgcacaaccaatgagaagggctggtgagggtgctgggactgagaggttgatggaaagattgaatattgggggcatgactcaggagaaagcactgaggaagaggtgctttggtgatggagttactggaactgcacgatgtgtgttcacatcagatgctgatagagacactcctcatcttcggacacaatcctcccgaaagaattatgctgatgcaagccacgtttctgctgtcattttgggtggaggcaagggagttggcctctttcctctgacaagcacaagggctacccccgctgttcctgttggaggatgttacaggcttattgacatccctatgagcaattgcttcaatagcggaataaataaaatatttgtgatgactcagttcaattctgcttctcttaatcgccatatccatcatacataccttggtggggggatcaactttactgatgggtctgtgcaggtattggctgctacacaaatgcctgacgaaccggctggatggttccagggtacagcagatgctatcagaaaatttatgtggatacttgaggatcattacaatcaaaacaacattgagcacgttgtaatcttgtgtggagatcagctttacaggatgaattacatggagcttgtgcagaaacatgtcgatgacaatgctgacattaccatatcatgtgctcccatcgacgggagccgagcttctgactatggactggtgaagtttgatgattcaggccgtgtgatccaatttcttgagaaaccagagggtgctgatttggaatcgatgaaagttgataccagcttcctcagctatgcaatagatgacaaacaaaagtatccctacattgcatcaatgggaatttatgtcttgaaaaaggatgttcttttagacattctgaagtcaaaatatgcacacctgcaagactttggatcagaaattctcccgagagctgttttagagcataatgtgaaggcatgtgtcttcacagaatattgggaggatattggaacaatcaaatcattttttgatgctaacttggccctcaccgagcagcctccaaagttcgaattttatgatccaaaaactcctttcttcacctcacctcgatacttacctccagcaagattggagaagtgcaagatcaaagatgcaataatctccgatggctgttcctttagtgaatgcaccattgagcattctgtcattggaatctcttcacgcgttagcatcgggtgtgaactcaaggataccatgatgatgggtgcagatcaatatgaaactgaagaagaaaggtcaaagctattgtttgaaggcaaggtcccaattggtataggcgagaacacaaagataaggaactgtatcatcgacatgaatgctagaattggcaggaacgtgatcattgcaaacactcagggggtccaagagtctgatcaccctgaagagggatactacataaggtctggaattgtggtgatcctgaagaatgcaaccatcaaggacgggaccgtcatataa

SEQ ID NO: 66 - DNA, 1557 bp, Oryza sativa
  misc_feature (1)..(1557): Modified AGPase large subunit cDNA
  misc_feature (472)..(474): G158N mutation
  misc_feature (487)..(489): D163G mutation
  misc_feature (1333)..(1335): T445R mutation
atgcaattcatgatgccattggatacgaatgcttgtgcacaaccaatgagaagggctggtgagggtgctgggactgagaggttgatggaaagattgaatattgggggcatgactcaggagaaagcactgaggaagaggtgctttggtgatggagttactggaactgcacgatgtgtgttcacatcagatgctgatagagacactcctcatcttcggacacaatcctcccgaaagaattatgctgatgcaagccacgtttctgctgtcattttgggtggaggcactggagttcaactctttcctctgacaagcacaagggctacccccgctgttcctgttggaggatgttacaggcttattgacatccctatgagcaattgcttcaatagcggaataaataaaatatttgtgatgactcagttcaattctgcttctcttaatcgccatatccatcatacataccttggtgggaacatcaactttactggcgggtctgtgcaggtattggctgctacacaaatgcctgacgaaccggctggatggttccagggtacagcagatgctatcagaaaatttatgtggatacttgaggatcattacaatcaaaacaacattgagcacgttgtaatcttgtgtggagatcagctttacaggatgaattacatggagcttgtgcagaaacatgtcgatgacaatgctgacattaccatatcatgtgctcccatcgacgggagccgagcttctgactatggactggtgaagtttgatgattcaggccgtgtgatccaatttcttgagaaaccagagggtgctgatttggaatcgatgaaagttgataccagcttcctcagctatgcaatagatgacaaacaaaagtatccctacattgcatcaatgggaatttatgtcttgaaaaaggatgttcttttagacattctgaagtcaaaatatgcacacctgcaagactttggatcagaaattctcccgagagctgttttagagcataatgtgaaggcatgtgtcttcacagaatattgggaggatattggaacaatcaaatcattttttgatgctaacttggccctcaccgagcagcctccaaagttcgaattttatgatccaaaaactcctttcttcacctcacctcgatacttacctccagcaagattggagaagtgcaagatcaaagatgcaataatctccgatggctgttcctttagtgaatgcaccattgagcattctgtcattggaatctcttcacgcgttagcatcgggtgtgaactcaaggataccatgatgatgggtgcagatcaatatgaaactgaagaagaaaggtcaaagctattgtttgaaggcaaggtcccaattggtataggcgagaacacaaagataaggaactgtatcatcgacatgaatgctagaattggcaggaacgtgatcattgcaaacactcagggggtccaagagtctgatcaccctgaagagggatactacataaggtctggaattgtggtgatcctgaagaatgcaaccatcaaggacgggaccgtcatataa

SEQ ID NO: 67 - DNA, 1557 bp, Oryza sativa
  misc_feature (1)..(1557): AGPase large subunit cDNA
atgcaattcatgatgccattggatacgaatgcttgtgcacaaccaatgagaagggctggtgagggtgctgggactgagaggttgatggaaagattgaatattgggggcatgactcaggagaaagcactgaggaagaggtgctttggtgatggagttactggaactgcacgatgtgtgttcacatcagatgctgatagagacactcctcatcttcggacacaatcctcccgaaagaattatgctgatgcaagccacgtttctgctgtcattttgggtggaggcactggagttcaactctttcctctgacaagcacaagggctacccccgctgttcctgttggaggatgttacaggcttattgacatccctatgagcaattgcttcaatagcggaataaataaaatatttgtgatgactcagttcaattctgcttctcttaatcgccatatccatcatacataccttggtggggggatcaactttactgatgggtctgtgcaggtattggctgctacacaaatgcctgacgaaccggctggatggttccagggtacagcagatgctatcagaaaatttatgtggatacttgaggatcattacaatcaaaacaacattgagcacgttgtaatcttgtgtggagatcagctttacaggatgaattacatggagcttgtgcagaaacatgtcgatgacaatgctgacattaccatatcatgtgctcccatcgacgggagccgagcttctgactatggactggtgaagtttgatgattcaggccgtgtgatccaatttcttgagaaaccagagggtgctgatttggaatcgatgaaagttgataccagcttcctcagctatgcaatagatgacaaacaaaagtatccctacattgcatcaatgggaatttatgtcttgaaaaaggatgttcttttagacattctgaagtcaaaatatgcacacctgcaagactttggatcagaaattctcccgagagctgttttagagcataatgtgaaggcatgtgtcttcacagaatattgggaggatattggaacaatcaaatcattttttgatgctaacttggccctcaccgagcagcctccaaagttcgaattttatgatccaaaaactcctttcttcacctcacctcgatacttacctccagcaagattggagaagtgcaagatcaaagatgcaataatctccgatggctgttcctttagtgaatgcaccattgagcattctgtcattggaatctcttcacgcgttagcatcgggtgtgaactcaaggataccatgatgatgggtgcagatcaatatgaaactgaagaagaaacttcaaagctattgtttgaaggcaaggtcccaattggtataggcgagaacacaaagataaggaactgtatcatcgacatgaatgctagaattggcaggaacgtgatcattgcaaacactcagggggtccaagagtctgatcaccctgaagagggatactacataaggtctggaattgtggtgatcctgaagaatgcaaccatcaaggacgggaccgtcatataa

SEQ ID NO: 68 - DNA, 4028 bp, Oryza sativa
  misc_feature (1)..(4028): Rice AGPase large subunit gDNA
atgcaattcatgatgccattggatacgaatgcttgtgcacaaccaatgagaagggctggtgagggtgctgggactgagaggttgatggaaagattgaatattgggggcatgactcaggagaaagcactgaggaagaggtgctttggtgatggagttactggaactgcacgatgtgtgttcacatcagatgctgatagagacactcctgtaagcatttaactctcttgtaaatttgtcgttttctttttattgaatttgttatttcacaattgtgttccacaggcacaatgcacttattcaagattatagagaattgtagctttcatcactgcattcgacctatggacatcttgatttactaataatacagtaagaacttatgggacaaattttataagatggttaaccagccagaagattggataattgatgtggtgtagttccatgcaatatacaattaattatttcgtccttcagttgactttccctcttagtttgtaggcgtattcatgaattgttgttccatatgtgtataaatgcacaggaagtgcgtatataccatccacttcaagtaatctaactgagcttaaaatttacagcatcttcggacacaatcctcccgaaagaattatgctgatgcaagccacgtttctgctgtcattttgggtggaggcactggagttcaactctttcctctgacaagcacaagggctacccccgctgtaagaaagtgaaacactatttatatctaatgttgactactgtactgtgcagataatagatttttaattgtggataacatacttagaacttaagaaaaagtttggaatctcttgtcagtttaaactttaaacaagtgctaattttaacttgctgttcttaatgtctgtcgcttaacttgattgagtgactgcaaatcaagttacaacaacatctggtatcatttggttatttcgacttgcttactatgtcatacggttactacattactatccatttggaactactctttctaggggcctagactaatgagacacactaatcaatatcagagtatcacatcaacacaactaagtattagatacaaggatgctaatcaacaagttatctacttttagaacttcatccgagaaaacatatgctagtagtatgttgtattttaccatgatgcctaaaagcatcaaatttaattctctcataaccttaagccatctaactttcactcatccaatcaggctatcaatgactaggagatagatgattcgaaatttcgttgtcaactgtgaatgaataatctaaattctatttttatttttctattacttaggttcctgttggaggatgttacaggcttattgacatccctatgagcaattgcttcaatagcggaataaataaaatatttgtgatgactcagttcaattctgcttctcttaatcgccatatccatcatacataccttggtggggggatcaactttactgatgggtctgtgcaggtaatttacctccatattcttgatatgccttagtggaaaccagttactagcagtagcaggtagcaacattaattgaactaacgttggcaatatataggtattggctgctacacaaatgcctgacgaaccggctggatggttccagggtacagcagatgctatcagaaaatttatgtggatacttgaggtaattaatttgtttcttccctgatattaatgggtaataattcttgcagaatgtaacttttatcatggatcataaaataccccttctattgcattcctgtccaggatcattacaatcaaaacaacattgagcacgttgtaatcttgtgtggagatcagctttacaggatgaattacatggagcttgtgcaggtgcattatgttgtcattctttttacttcatgtattcttgattttcgattgagcaatactttgacatgtaatacttgcagaaacatgtcgatgacaatgctgacattaccatatcatgtgctcccatcgacgggaggtaaattagttcttgtgcatcctgattctatgagcgtgccttatccaacataagatgcatgtgttctaattttcttcttttttattgggaagccgagcttctgactatggactggtgaagtttgatgattcaggccgtgtgatccaatttcttgagaaaccagagggtgctgatttggaatcgatggttagtaattgtcatctttactgttctcttgagaaatcttttctttagttcatctgcacggttggtctgtcagaaataaatatctatttctgcataatgctgcactaaatcaactgatcactgcagaaagttgataccagcttcctcagctatgcaatagatgacaaacaaaagtatccctacattgcatcaatgggaatttatgtcttgaaaaaggatgttcttttagacattctgaagtaagtgtcacccatttgtgatagttcggtccacttcctatgtcttctgataattcgtattcttaggtcaaaatatgcacacctgcaagactttggatcagaaattctcccgagagctgttttagagcataatgtgaaggtaagttccgaacaactgtaatccgggtttcctacataatctccatctattgcttttgcatattcaatggtttagttaatgctacctccacccacagtggacatcaacacagtctgaaaacacaagtttgaccagtagtttccattgtgatatacttatagagtatgataaatttacagtctgaatatccttagtgttttgtggttgctggagcgctgggagtaactagcatgacaacacccagagcaatatacagatcaatgagctttttacattttgcctactctatataacttgaactagcactatattgttttaggcatgtgtcttcacagaatattgggaggatattggaacaatcaaatcattttttgatgctaacttggccctcaccgagcaggtattgccacatatgtattctgtacaactgatgcactctttaacatattgcgagacaccttgtacagtaatacccaaagatgtcttacagagtctaatgatttacccattattcttcagttaacttactgctaacaccaatttgtatacaaaagttgtctgaacatgtttctccgttccagcctccaaagttcgaattttatgatccaaaaactcctttcttcacctcacctcgatacttacctccagcaagattggagaagtgcaaggtatgagtcatgtaaaccacagttgttgcttcagatcacagcaaatgcaatttaaattttggatcttttttttgtgcagatcaaagatgcaataatctccgatggctgttcctttagtgaatgcaccattgagcattctgtcattggaatctcttcacgcgttagcatcgggtgtgaactcaaggtactttatcaagacataaagctcatagtttggacattcaatgcatcaaaatttgatgtcttattaagattagattgattcataggtactgtttggaaactaccaattattctcatgtagttttgcaattctaaaattcacaggataccatgatgatgggtgcagatcaatatgaaactgaagaagaaacttcaaagctattgtttgaaggcaaggtcccaattggtataggcgagaacacaaagataaggtgagccacatctggactaatgcttacatattactgtccttacctggcaatatcgtgtttccaaatgtatgagaccttcgtttcatgaattttaggaactgtatcatcgacatgaatgctagaattggcaggaacgtgatcattgcaaacactcaggtgagcaagtatatgtatttaactactccaaagtcgatgaactcatcttttataaacacttggtaaccacacgagttcactgcgtatgatagggggtccaagagtctgatcaccctgaagagggatactacataaggtctggaattgtggtgatcctgaagaatgcaaccatcaaggacgggaccgtcatataa

SEQ ID NO: 69 - DNA, 3801 bp, Prevotella bryantii B14
  misc_feature (1)..(3801): Cpf1 nuclease
atggccccaaagaagaaacggaaggttatgcaaataaacaacctcaagatcatatatatgaagttcacagatttcaccggcctgtatagcttgtccaagactctcaggttcgagttgaagccgatcgggaagacactcgaaaacataaaaaaggcgggacttttggagcaagatcagcatagggcggactcctacaagaaggtgaagaaaatcattgatgaatatcacaaggcttttatagaaaagtcgctttctaatttcgaactcaaataccaatcggaagacaagctcgactcgttggaagaatatctgatgtactactcgatgaagagaattgaaaagaccgaaaaagataagttcgccaaaatccaggacaacctcagaaaacagatcgcggaccaccttaaaggcgatgagtcttacaaaacaatattttccaaggatttgatcaggaagaatttgccggacttcgtcaagagcgacgaggaacggactttgattaaggagtttaaggacttcaccacgtactttaaagggttctacgaaaatagagagaatatgtacagcgcggaggataaaagcaccgccatctcccatagaatcatccacgagaatctccctaagtttgtggataacataaacgcgttctccaaaataatcctcatcccagagctgcgggagaagctcaatcagatataccaggacttcgaagagtacctgaacgtggagtcaattgatgaaatattccatcttgactacttcagcatggttatgacacaaaagcagatcgaagtctataacgccattattggcgggaagtctaccaacgacaagaagatacaggggcttaatgagtatattaatctgtataaccaaaagcacaaagattgcaagttgccaaagctgaaactgttgttcaagcagattcttagcgaccggatagcgatctcttggctcccggataacttcaaggatgatcaggaggcgcttgactcaatagatacatgctacaagaatctcctgaatgacgggaacgtgcttggggaaggaaatctgaagctgctgctcgagaacatagatacatacaatctgaagggaatttttattagaaatgatctccaactgacagatatttcacagaagatgtacgcttcttggaatgtcattcaagatgcagttattcttgatttgaaaaagcaggtttcccgcaagaagaaggagagcgcggaagattacaacgatagactgaagaagctctatacgagccaggagagcttttcgatacagtacctcaacgactgcttgagggcgtacgggaaaacagaaaacattcaagattactttgcgaaactgggggcggtcaacaacgagcacgagcagacgatcaacttgtttgcacaagttaggaacgcctatacttccgttcaagccatcctcacaacaccgtaccctgagaatgccaacttggctcaggacaaagagaccgtcgcacttatcaagaacctccttgattcactgaagcgccttcaaaggtttatcaaacctctcctcggcaagggcgatgagagcgacaaggacgagcgcttctacggagacttcactcccctttgggagacgctcaaccaaataaccccattgtataatatggtgagaaactacatgacaaggaaaccttattcgcaggagaaaataaagctcaattttgaaaactctaccttgcttgggggatgggatctgaacaaagagcacgataatactgcaatcatacttcgcaagaatggtctttactacctggctatcatgaaaaaaagcgccaacaagatcttcgataaggacaaattggacaactctggcgactgttacgaaaaaatggtgtataaactgctcccaggcgccaacaaaatgcttcccaaggttttcttttccaagtcgcgcatcgacgagttcaaaccttctgagaacataatcgagaactacaagaagggaactcacaaaaaaggggccaacttcaatctcgcagattgccacaacttgatcgatttctttaaatcctctatatcgaagcatgaagactggtccaagttcaactttcatttcagcgacacctcatcttatgaggacctttcagacttctacagagaggtggagcaacaagggtactcaatctcattttgcgatgtcagcgtggagtacattaacaagatggttgagaagggggacctttacctcttccaaatctacaataaggatttctcggaattcagcaagggcacgcctaatatgcacacgctctattggaacagcctcttctcgaaagagaacctgaataatataatatacaaactcaatggccaagctgagattttcttcaggaagaagtcactcaattacaagcggccgacacacccggcgcatcaagctattaagaacaagaacaagtgtaatgagaaaaaagaatccatcttcgattatgacctcgtcaaggataagcgctacaccgttgataaatttcagtttcacgttccaattaccatgaactttaagtctacaggaaataccaatataaaccaacaggtcattgactatcttcgcacggaggacgatacgcacatcatcggaatcgatcggggggagcggcacctgctttatttggtggttatcgactcccatgggaaaatcgtggaacagttcaccctcaatgagattgtgaacgagtacggcggaaacatttatagaacgaattaccacgacttgctggacacgagagagcaaaacagggagaaagcgagggagtcatggcagacaatcgagaacataaaagaattgaaagagggctatatctctcaagtgattcataaaataacggatctcatgcaaaagtaccatgcagtcgtcgtgctcgaggatctcaacatgggattcatgaggggaaggcagaaggtggagaagcaggtctatcaaaaatttgaagaaatgctgatcaataaacttaattatctggtcaacaaaaaggctgatcagaatagcgctggagggctgttgcacgcctaccagctgacatcaaagttcgagtccttccagaaacttggcaagcagtctggttttcttttttacattccagcctggaatacctccaaaattgatcctgtcaccggatttgtgaacttgtttgacaccaggtacgagagcatagacaaggcgaaggcattctttggaaagttcgattccataaggtataatgcggacaaagactggttcgagtttgcgttcgactataacaacttcacaactaaagcagaggggactagaacaaattggacgatctgtacatacggttcaaggatcaggaccttcaggaatcaagcgaagaattctcaatgggacaacgaggagatcgatctgacaaaagcctacaaggcttttttcgccaaacatggaatcaatatctatgataatatcaaggaggccatagccatggaaactgagaaaagcttctttgaggacctgcttcatctgcttaaattgactctgcagatgcggaactccattaccggcacaaccactgattatctcatttcgccagtccatgactctaaaggaaacttctacgatagcaggatctgtgataatagccttcctgccaacgcggacgcgaacggcgcgtataatattgcccgcaaaggattgatgctcatacagcaaatcaaggattcgacgagctcgaaccgctttaagttctcacctattacgaataaagattggctcatttttgcacaagagaagccgtacttgaatgactga

SEQ ID NO: 70 - protein, 1266 aa, Prevotella bryantii B14
  MISC_FEATURE (1)..(1266): Cpf1 nuclease
MAPKKKRKVMQINNLKIIYMKFTDFTGLYSLSKTLRFELKPIGKTLENIKKAGLLEQDQHRADSYKKVKKIIDEYHKAFIEKSLSNFELKYQSEDKLDSLEEYLMYYSMKRIEKTEKDKFAKIQDNLRKQIADHLKGDESYKTIFSKDLIRKNLPDFVKSDEERTLIKEFKDFTTYFKGFYENRENMYSAEDKSTAISHRIIHENLPKFVDNINAFSKIILIPELREKLNQIYQDFEEYLNVESIDEIFHLDYFSMVMTQKQIEVYNAIIGGKSTNDKKIQGLNEYINLYNQKHKDCKLPKLKLLFKQILSDRIAISWLPDNFKDDQEALDSIDTCYKNLLNDGNVLGEGNLKLLLENIDTYNLKGIFIRNDLQLTDISQKMYASWNVIQDAVILDLKKQVSRKKKESAEDYNDRLKKLYTSQESFSIQYLNDCLRAYGKTENIQDYFAKLGAVNNEHEQTINLFAQVRNAYTSVQAILTTPYPENANLAQDKETVALIKNLLDSLKRLQRFIKPLLGKGDESDKDERFYGDFTPLWETLNQITPLYNMVRNYMTRKPYSQEKIKLNFENSTLLGGWDLNKEHDNTAIILRKNGLYYLAIMKKSANKIFDKDKLDNSGDCYEKMVYKLLPGANKMLPKVFFSKSRIDEFKPSENIIENYKKGTHKKGANFNLADCHNLIDFFKSSISKHEDWSKFNFHFSDTSSYEDLSDFYREVEQQGYSISFCDVSVEYINKMVEKGDLYLFQIYNKDFSEFSKGTPNMHTLYWNSLFSKENLNNIIYKLNGQAEIFFRKKSLNYKRPTHPAHQAIKNKNKCNEKKESIFDYDLVKDKRYTVDKFQFHVPITMNFKSTGNTNINQQVIDYLRTEDDTHIIGIDRGERHLLYLVVIDSHGKIVEQFTLNEIVNEYGGNIYRTNYHDLLDTREQNREKARESWQTIENIKELKEGYISQVIHKITDLMQKYHAVVVLEDLNMGFMRGRQKVEKQVYQKFEEMLINKLNYLVNKKADQNSAGGLLHAYQLTSKFESFQKLGKQSGFLFYIPAWNTSKIDPVTGFVNLFDTRYESIDKAKAFFGKFDSIRYNADKDWFEFAFDYNNFTTKAEGTRTNWTICTYGSRIRTFRNQAKNSQWDNEEIDLTKAYKAFFAKHGINIYDNIKEAIAMETEKSFFEDLLHLLKLTLQMRNSITGTTTDYLISPVHDSKGNFYDSRICDNSLPANADANGAYNIARKGLMLIQQIKDSTSSNRFKFSPITNKDWLIFAQEKPYLND

SEQ ID NO: 71 - DNA, 780 bp, Cauliflower Mosaic Virus
  misc_feature (1)..(780): 2X35S promoter
atggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgaaatcaccagtctctctctacaaatctatctct

SEQ ID NO: 72 - DNA, 44 bp, Artificial
  synthetic
  misc_feature (1)..(44): gRNA
  misc_feature (1)..(20): Cpf1-interacting portion
  misc_feature (21)..(44): AGPase targeting region
gaatttctactgttgtagattaggggcctagactaatgagacac

SEQ ID NO: 73 - DNA, 516 bp, Oryza sativa
  misc_feature (1)..(516): U6 promoter
tttgtgaaagttgaattacggcatagccgaaggaataacagaatcgtttcacactttcgtaacaaaggtcttcttatcatgtttcagacgatggaggcaaggctgatcaaagtgatcaagcacataaacgcatttttttaccatgtttcactccataagcgtctgagattatcacaagtcacgtctagtagtttgatggtacactagtgacaatcagttcgtgcagacagagctcatacttgactacttgagcgattacaggcgaaagtgtgaaacgcatgtgatgtgggctgggaggaggagaatatatactaatgggccgtatcctgatttgggctgcgtcggaaggtgcagcccacgcgcgccgtaccgcgcgggtggcgctgctacccactttagtccgttggatggggatccgatggtttgcgcggtggcgttgcgggggatgtttagtaccacatcggaaaccgaaagacgatggaaccagcttataaacccgcgcgctgtagtcagctt

SEQ ID NO: 74 - DNA, 1108 bp, Oryza sativa
  misc_feature (1)..(1108): repair donor template targeting AGPase large subunit exons 2 and 3
  misc_feature (118)..(118): introduced A>T mutation
  misc_feature (170)..(172): introduced ACT>AAG T95K mutation
  misc_feature (179)..(181): introduced CAA>GGC Q98G mutation
  misc_feature (223)..(223): introduced A>C mutation
  misc_feature (235)..(252): primer pad 1
  misc_feature (282)..(282): introduced T>G mutation
  misc_feature (410)..(410): introduced A>C mutation
  misc_feature (507)..(507): introduced T>G mutation
  misc_feature (509)..(532): sequence targeted by gRNA
  misc_feature (627)..(627): introduced A>C mutation
  misc_feature (781)..(798): primer pad 2
  misc_feature (811)..(811): introduced T>G mutation
  misc_feature (964)..(966): introduced GGG>AAC G158N mutation
  misc_feature (979)..(981): introduced GAT>GGC D163G mutation
  misc_feature (1030)..(1030): introduced A>C mutation
gcgatcgcattgttgttccatatgtgtataaatgcacaggaagtgcgtatataccatccacttcaagtaatctaactgagcttaaaatttacagcatcttcggacacaatcctcccgtaagaattatgctgatgcaagccacgtttctgctgtcattttgggtggaggcaagggagttggcctctttcctctgacaagcacaagggctacccccgctgtaagcaagtgaaacacgacctcaacctagatgtcactactgtactgtgcagataatagattttgaattgtggataacatacttagaacttaagaaaaagtttggaatctcttgtcagtttaaactttaaacaagtgctaattttaacttgctgttcttaatgtctgtcgcttaacttgattgagtgactgccaatcaagttacaacaacatctggtatcatttggttatttcgacttgcttactatgtcatacggttactacattactatccatttggaactactcttgctaggggcctagactaatgagacacactaatcaatatcagagtatcacatcaacacaactaagtattagatacaaggatgctaatcaacaagttatctacttttagaacttcatccgagcaaacatatgctagtagtatgttgtattttaccatgatgcctaaaagcatcaaatttaattctctcataaccttaagccatctaactttcactcatccaatcaggctatcaatgactaggagatagatgattcgaaatttcgttgtcaactgtgtacctacgtagtggtacctatttttattttgctattacttaggttcctgttggaggatgttacaggcttattgacatccctatgagcaattgcttcaatagcggaattaataaaatatttgtgatgactcagttcaattctgcttctcttaatcgccatatccatcatacataccttggtgggaacatcaactttactggcgggtctgtgcaggtaatttacctccatattcttgatatgccttagtggcaaccagttactagcagtagcaggtagcaacattaattgaactaacgttggcaatatataggtattggctggcggccgc

SEQ ID NO: 75 - DNA, 1033 bp, Escherichia coli
  misc_feature (1)..(1033): hygromycin resistance gene
atgagatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggagtttagcgagagcctgacctattgcatctcccgccgttcacagggtgtcacgttgcaagacctgcctgaaaccgaactgcccgctgttctacaaccggtcgcggaggctatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccacgactccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaagaaatag

SEQ ID NO: 76 - protein, 341 aa, Escherichia coli
  MISC_FEATURE (1)..(341): hygromycin resistance protein
MKKPELTATSVEKFLIEKFDSVSDLMQLSEGEESRAFSFDVGGRGYVLRVNSCADGFYKDRYVYRHFASAALPIPEVLDIGEFSESLTYCISRRSQGVTLQDLPETELPAVLQPVAEAMDAIAAADLSQTSGFGPFGPQGIGQYTTWRDFICAIADPHVYHWQTVMDDTVSASVAQALDELMLWAEDCPEVRHLVHADFGSNNVLTDNGRITAVIDWSEAMFGDSQYEVANIFFWRPWLACMEQQTRYFERRHPELAGSPRLRAYMLRIGLDQLYQSLVDGNFDDAAWAQGRCDAIVRSGAGTVGRTQIARRSAAVWTDGCVEVLADSGNRRPSTRPRAKK

SEQ ID NO: 77 - DNA, 1993 bp, Zea mays
  misc_feature (1)..(1993): ZmUbi promoter
cagcaagcttctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatactactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcacggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgtta

SEQ ID NO: 78 - DNA, 1092 bp, Oryza sativa
  misc_feature (1)..(1092): Native rice AGPase large subunit coding sequence (partial)
attgttgttccatatgtgtataaatgcacaggaagtgcgtatataccatccacttcaagtaatctaactgagcttaaaatttacagcatcttcggacacaatcctcccgaaagaattatgctgatgcaagccacgtttctgctgtcattttgggtggaggcactggagttcaactctttcctctgacaagcacaagggctacccccgctgtaagaaagtgaaacactatttatatctaatgttgactactgtactgtgcagataatagatttttaattgtggataacatacttagaacttaagaaaaagtttggaatctcttgtcagtttaaactttaaacaagtgctaattttaacttgctgttcttaatgtctgtcgcttaacttgattgagtgactgcaaatcaagttacaacaacatctggtatcatttggttatttcgacttgcttactatgtcatacggttactacattactatccatttggaactactctttctaggggcctagactaatgagacacactaatcaatatcagagtatcacatcaacacaactaagtattagatacaaggatgctaatcaacaagttatctacttttagaacttcatccgagaaaacatatgctagtagtatgttgtattttaccatgatgcctaaaagcatcaaatttaattctctcataaccttaagccatctaactttcactcatccaatcaggctatcaatgactaggagatagatgattcgaaatttcgttgtcaactgtgaatgaataatctaaattctatttttatttttctattacttaggttcctgttggaggatgttacaggcttattgacatccctatgagcaattgcttcaatagcggaataaataaaatatttgtgatgactcagttcaattctgcttctcttaatcgccatatccatcatacataccttggtggggggatcaactttactgatgggtctgtgcaggtaatttacctccatattcttgatatgccttagtggaaaccagttactagcagtagcaggtagcaacattaattgaactaacgttggcaatatataggtattggctg

SEQ ID NO: 79 - DNA, 26 bp, Artificial
  primer sequence
  misc_feature (1)..(26): primer sequence annealing to gDNA upstream of rice AGPase large subunit repair template target
cctcttagtttgtaggcgtattcatg

SEQ ID NO: 80 - DNA, 23 bp, Artificial
  primer sequence
  misc_feature (1)..(23): primer sequence annealing to gDNA downstream from targeted sequence in rice AGPase large subunit
ttctgatagcatctgctgtaccc

SEQ ID NO: 81 - DNA, 1100 bp, Oryza sativa
  misc_feature (1)..(1100): Partial sequence of AGPase large subunit gene in rice callus piece #14
gcgatcgcattgttgttccatatgtgtataaatgcacaggaagtgcgtatataccatccacttcaagtaatctaactgagcttaaaatttacagcatcttcggacacaatcctcccgtaagaattatgctgatgcaagccacgtttctgctgtcattttgggtggaggcaagggagttggcctctttcctctgacaagcacaagggctacccccgctgtaagcaagtgaaacacgacctcaacctagatgtcactactgtactgtgcagataatagattttgaattgtggataacatacttagaacttaagaaaaagtttggaatctcttgtcagtttaaactttaaacaagtgctaattttaacttgctgttcttaatgtctgtcgcttaacttgattgagtgactgccaatcaagttacaacaacatctggtatcatttggttatttcgacttgcttactatgtcatacggttactacattactatccatttggaactactcttgctaggggcctagactaatgagacacactaatcaatatcagagtatcacatcaacacaactaagtattagatacaaggatgctaatcaacaagttatctacttttagaacttcatccgagcaaacatatgctagtagtatgttgtattttaccatgatgcctaaaagcatcaaatttaattctctcataaccttaagccatctaactttcactcatccaatcaggctatcaatgactaggagatagatgattcgaaatttcgttgtcaactgtgtacctacgtagtggtacctatttttattttgctattacttaggttcctgttggaggatgttacaggcttattgacatccctatgagcaattgcttcaatagcggaattaataaaatatttgtgatgactcagttcaattctgcttctcttaatcgccatatccatcatacataccttggtggggggatcaactttactgatgggtctgtgcaggtaatttacctccatattcttgatatgccttagtggaaaccagttactagcagtagcaggtagcaacattaattgaactaacgttggcaatatataggtattggctg

SEQ ID NO: 82 - DNA, 23 bp, Artificial
  primer sequence
  misc_feature (1)..(23): primer sequence annealing to primer pad in repair donor template
tagtgacatctaggttgaggtcg

SEQ ID NO: 83 - DNA, 22 bp, Artificial
  primer sequence
  misc_feature (1)..(22): primer sequence that anneals with primer pad
  misc_feature (1)..(22): primer sequence that anneals with primer pad in repair donor template
tgtgtacctacgtagtggtacc

(19) United States
(12) Patent Application Publication
(10) Pub. No.: US 2021/0002658 A1
(43) Pub. Date: Jan. 7, 2021

(54) MODIFIED AGPASE LARGE SUBUNIT SEQUENCES AND METHODS FOR DETECTION OF PRECISE GENOME EDITS

(71) Applicant: BENSON HILL, INC., St. Louis, MO (US)

(72) Inventors: Matthew Begemann, St. Louis, MO (US); Emma January, St. Louis, MO (US)

(73) Assignee: BENSON HILL, INC., St. Louis, MO (US)

(21) Appl. No.: 16/955,379

(22) PCT Filed: Dec. 18, 2018
(86) PCT No.: PCT/IB2018/060249

§ 371 Date: Jun. 18, 2020

Related U.S. Application Data
(60) Provisional application No. 62/607,644, filed on Dec. 19, 2017.
(60) Provisional application No. 62/723,626, filed on Aug. 28, 2018.

(51) Int. Cl. C12N 15/82; C12N 9/12; C12Q 1/6806; C12N 15/10

(52) U.S. Cl. CPC C12N 15/8213; C12N 9/1241; C12Q 1/6806; C12Q 2600/156; C12Y 207/07027; C12Q 2531/113; C12N 15/10

(57) ABSTRACT

Compositions and methods for improving plant growth are provided herein. Polynucleotides encoding modified AGPase large subunit proteins, polypeptides encompassing modified AGPase large subunit proteins, methods of producing modified polynucleotides encoding modified AGPase large subunit proteins, and expression constructs for expressing genes of interest whose expression may improve agronomic properties including but not limited to crop yield, biotic and abiotic stress tolerance, and early vigor, plants comprising the polynucleotides, polypeptides, and expression constructs, and methods of producing plants comprising the modified AGPase large subunit genes of the invention or encoding the AGPase large subunit proteins of the invention are also provided. Compositions and methods for the ready detection of precise base changes are also provided herein. Compositions include primer pads as part of a repair donor template, and methods for the detection of precise base changes include PCR detection using one or more primers designed to anneal with a primer pad sequence.

### FIELD OF THE INVENTION

The invention is drawn to modified ADP-glucose pyrophosphorylase (AGPase) large subunit sequences, methods for producing the same, and methods for expression of modified AGPase large subunit sequences in plants. Further, the invention is drawn to primer pads for ready detection of precise genome edits and methods for use of the same.

### REFERENCE TO A SEQUENCE LISTING SUBMITTED AS A TEXT FILE VIA EFS-WEB

The official copy of the sequence listing is submitted concurrently with the specification as a text file via EFS-Web, in compliance with the American Standard Code for Information Interchange (ASCII), with a file name of BHP019P3 sequence listing_ST25.txt, a creation date of Nov. 28, 2018, and a size of 387 Kb. The sequence listing filed via EFS-Web is part of the specification and is hereby incorporated in its entirety by reference herein.

### BACKGROUND OF THE INVENTION

The ever-increasing world population and the dwindling supply of arable land available for agriculture fuels research towards developing plants with increased biomass and yield. Conventional means for crop and horticultural improvements utilize selective breeding techniques to identify plants having desirable characteristics resulting, e.g., from modifications in protein coding sequences and/or in the expression levels or expression patterns associated with protein coding sequences. However, such selective breeding techniques have several drawbacks, namely that these techniques are typically labor intensive and result in plants that often contain heterogeneous genetic components that may not always result in the desirable trait being passed on from parent plants. Advances in molecular biology provide means to precisely modify the germplasm of plants. Genetic engineering of plants entails the isolation and manipulation of genetic material (typically in the form of DNA or RNA) and the subsequent introduction of that genetic material into a plant. Such technology has the capacity to deliver crops or plants having various improved economic, agronomic or horticultural traits.

Traits of interest include plant biomass and yield. Yield is normally defined as the measurable produce of economic value from a crop. This may be defined in terms of quantity and/or quality. Yield is directly dependent on several factors, for example, the number and size of the organs, plant architecture (for example, the number of branches), seed production, leaf senescence and more. Root development, nutrient uptake, stress tolerance, photosynthetic carbon assimilation rates, and early vigor may also be important factors in determining yield. Optimizing the abovementioned factors may therefore contribute to increasing crop yield.

An increase in seed yield is a particularly important trait since the seeds of many plants are important for human and animal consumption. Crops such as corn, rice, wheat, canola and soybean account for over half the total human caloric intake, whether through direct consumption of the seeds themselves or through consumption of meat products raised on processed seeds. They are also a source of sugars, oils and many kinds of metabolites used in industrial processes. Seeds contain an embryo (the source of new shoots and roots) and an endosperm (the source of nutrients for embryo growth during germination and during early growth of seedlings). The development of a seed involves many genes, and requires the transfer of metabolites from the roots, leaves and stems into the growing seed. The endosperm, in particular, assimilates the metabolic precursors of carbohydrates, oils and proteins and synthesizes them into storage macromolecules to fill out the grain. An increase in plant biomass is important for forage crops like alfalfa, silage corn and hay. Many genes are involved in the metabolic pathways that contribute to plant growth and development. Modifying the coding sequence and/or modulating the expression of one or more such genes in a plant can produce a plant with improved growth and development relative to a control plant, but often can produce a plant with impaired growth and development relative to a control plant. Therefore, methods to improve plant growth and development are needed.

AGPase genes have been studied as a means to improve crop yield because it is known that AGPase activity can be rate-limiting for starch production and that AGPase enzymes in many plant species are heat-labile. In higher plants, the functional AGPase holoenzyme typically comprises two large subunits and two small subunits. Modified versions of both AGPase large subunit and AGPase small subunit proteins have been generated that show improved enzyme kinetics and/or improved thermotolerance relative to unmodified AGPase proteins. Genome editing approaches offer an opportunity to precisely modify AGPase large subunit and/or AGPase small subunit protein-encoding sequences to encode modified proteins with desirable biochemical properties, e.g., improved enzyme kinetics and/or improved thermotolerance.

One method for modifying AGPase genes and other genes of interest in crop plants and in other organisms is the use of genome editing and particularly the use of homology-directed repair (HDR) mediated genome edits for precise modification of predetermined DNA sequences. Detection of these HDR-mediated modifications may be difficult, particularly when only one or a few mutations are present as compared with the native sequence, but this detection is critical for the successful deployment of a genome editing strategy. The present invention describes primer pads, providing a novel method for ready molecular identification of the desired genome editing events regardless of the editing system used.

### SUMMARY OF THE INVENTION

Compositions comprising modified AGPase large subunit sequences, methods for making such modified AGPase large subunit sequences, and methods for their use are provided. The methods result in engineered, non-naturally occurring coding sequences for mutant AGPase large subunit proteins. Methods include genome editing approaches to specifically modify AGPase large subunit gene sequences. The invention also encompasses constructs comprising a site-specific nuclease, or polynucleotide encoding a site-specific nuclease, along with guide molecule(s) and repair donor template(s) to produce the desired site-specific modifications. In other embodiments, a plant can be transformed with a construct comprising a mutant AGPase large subunit gene for expression in the plant. Compositions further comprise plants, plant seeds, plant organs, plant cells, and other plant parts that comprise modified AGPase large subunit coding sequences. The modified AGPase large subunit sequences of the invention may be produced by modifying a native AGPase large subunit sequence, or alternatively, may be an AGPase large subunit sequence that is heterologous to the plant of interest. Transformed plant cells, tissues, and plants are provided.

The invention also encompasses primer pads and methods to use such primer pads to enable ready detection of precise site-specific modifications. The methods and compositions allow one to identify genome edits that have been introduced into a genome regardless of the gene editing system used. Furthermore, the primer pad methods can be used in any prokaryotic or eukaryotic cell.

Embodiments of the invention include:
1. A modified AGPase large subunit protein comprising one or more non-native amino acid residues selected from the group consisting of:
   a. Lysine at position 249 and glycine at position 252;
   b. Asparagine at position 312 and glycine at position 317; and
   c. Arginine at position 599, where positions 249, 252, 312, 317, and 599 correspond to the amino acid numbering of the maize sh2 protein (SEQ ID NO:3).
2. A modified AGPase large subunit protein comprising one or more non-native amino acid residues selected from the group consisting of:
   a. Lysine at position 95 and glycine at position 98;
   b. Asparagine at position 158 and glycine at position 163; and
   c. Arginine at position 445,
where positions 95, 98, 158, 163, and 445 correspond to the amino acid numbering of the Oryza sativa AGPase large subunit protein (SEQ ID NO:24).
3. A polynucleotide sequence encoding the modified AGPase large subunit protein of any one of embodiments 1 and 2.
4. The modified AGPase large subunit protein of either one of embodiments 1 and 2 wherein said non-native amino acid residues comprise a lysine at position 249 and glycine at position 252, wherein positions 249 and 252 correspond to the amino acid numbering of SEQ ID NO:3, or wherein said non-native amino acid residues comprise a lysine at position 95 and glycine at position 98, wherein positions 95 and 98 correspond to the amino acid numbering of SEQ ID NO:24.
5. A polynucleotide encoding the AGPase large subunit of embodiment 4.
6. The modified AGPase large subunit protein of embodiment 4 wherein said AGPase large subunit protein shares at least 80% identity with SEQ ID NO:6 or 49.
7. The modified AGPase large subunit of embodiment 4 wherein said AGPase large subunit protein is encoded by a polynucleotide having at least 70% identity with a sequence selected from SEQ ID NOs:5, 43, 55, or 61.
8. The modified AGPase large subunit protein of embodiment 4 wherein said AGPase large subunit protein comprises the sequence set forth in SEQ ID NO:6 or 49.
9. The modified AGPase large subunit protein of embodiment 8, wherein said AGPase large subunit protein is encoded by a polynucleotide that comprises a sequence selected from SEQ ID NOs:5, 43, 55, or 61.
10. The modified AGPase large subunit protein of either one of embodiments 1 and 2 wherein said non-native amino acid residues comprise an asparagine at position 312 and glycine at position 317, wherein positions 312 and 317 correspond to the amino acid numbering of SEQ ID NO:3, or wherein said non-native amino acid residues comprise an asparagine at position 158 and glycine at position 163, wherein positions 158 and 163 correspond to the amino acid numbering of SEQ ID NO:24.
11. A polynucleotide encoding the modified AGPase large subunit protein of embodiment 10.
12. The modified AGPase large subunit protein of embodiment 4 wherein said AGPase large subunit protein shares at least 80% identity with SEQ ID NO:8 or 50.
13. The modified AGPase large subunit protein of embodiment 12, wherein said AGPase large subunit protein is encoded by a polynucleotide that shares at least 70% identity with a sequence selected from SEQ ID NOs:7, 44, 56, or 62.
14. The modified AGPase large subunit protein of embodiment 4 wherein said AGPase large subunit protein comprises SEQ ID NO:8 or 50.
15. The modified AGPase protein of embodiment 14, wherein said AGPase large subunit protein is encoded by a polynucleotide that comprises a sequence selected from SEQ ID NOs:7, 44, 56, or 62.
16. The modified AGPase large subunit protein of either one of embodiments 1 and 2 wherein said non-native amino acid residues comprise a lysine at position 249, glycine at position 252, asparagine at position 312, and glycine and position 317, wherein positions 249, 252, 312, and 317 correspond to the numbering of SEQ ID NO:3, or wherein said non-native amino acid residues comprise a lysine at position 95, glycine at position 98, asparagine at position 158, and glycine and position 163, wherein positions 95, 98, 158, and 163 correspond to the numbering of SEQ ID NO:24.
17. A polynucleotide encoding the modified AGPase large subunit protein of embodiment 16.
18. The modified AGPase large subunit protein of embodiment 4 wherein said AGPase large subunit protein shares at least 80% identity with SEQ ID NO:10 or 51.
19. The modified AGPase large subunit of embodiment 18, wherein said AGPase large subunit protein is encoded by a polynucleotide that shares at least 70% identity with a sequence selected from SEQ ID NOs:9, 45, 57, or 63.
20. The modified AGPase large subunit protein of embodiment 4 wherein said AGPase large subunit protein comprises SEQ ID NO:10 or 51.
21. The modified AGPase large subunit of embodiment 20, wherein said AGPase large subunit protein is encoded by a polynucleotide that comprises a sequence selected from SEQ ID NOs:9, 45, 57, or 63.
22. The modified AGPase large subunit protein of either one of embodiments 1 and 2 wherein said non-native amino acid residues comprise a lysine at position 249, glycine at position 252, asparagine at position 312, glycine at position 317, and arginine at position 599, wherein said positions 249, 252, 312, 317, and 599 correspond to the amino acid numbering of SEQ ID NO:3, or wherein said non-native amino acid residues comprise a lysine at position 95, glycine at position 98, asparagine at position 158, glycine at position 163, and arginine at position 445, wherein said positions 95, 98, 158, 163, and 445 correspond to the amino acid numbering of SEQ ID NO:24.
23. A polynucleotide encoding the modified AGPase large subunit protein of embodiment 22.
24. The modified AGPase large subunit protein of embodiment 4 wherein said AGPase large subunit protein shares at least 80% identity with SEQ ID NO:12 or 52.
25. The modified AGPase large subunit protein of embodiment 24, wherein said modified AGPase large subunit protein is encoded by a polynucleotide that shares at least 70% identity with a sequence selected from the group consisting of SEQ ID NOs:11, 46, 58, and 64.
26. The modified AGPase large subunit protein of embodiment 4 wherein said AGPase large subunit protein comprises SEQ ID NO:12 or 52.
27. The modified AGPase large subunit protein of embodiment 26, wherein said modified AGPase large subunit protein is encoded by a polynucleotide that comprises a sequence selected from the group consisting of SEQ ID NOs:11, 46, 58, and 64.
28. The modified AGPase large subunit protein of either one of embodiments 1 and 2 wherein said non-native amino acid residues comprise a lysine at position 249, glycine at position 252, and arginine at position 599, wherein said positions 249, 252, and 599 correspond to the numbering of SEQ ID NO:3, or wherein said non-native amino acid residues comprise a lysine at position 95, glycine at position 98, and arginine at position 445, wherein said positions 95, 98, and 445 correspond to the numbering of SEQ ID NO:24.
29. A polynucleotide encoding the modified AGPase large subunit protein of embodiment 26.
30. The modified AGPase large subunit protein of embodiment 4 wherein said AGPase large subunit protein shares at least 80% identity with SEQ ID NO:14 or 53.
31. The modified AGPase large subunit protein of embodiment 30, wherein said modified AGPase large subunit protein is encoded by a polynucleotide that shares at least 70% identity with a sequence selected from the group consisting of SEQ ID NOs:13, 47, 59, and 65.
32. The modified AGPase large subunit protein of embodiment 4 wherein said AGPase large subunit protein comprises SEQ ID NO:14 or 53.
33. The modified AGPase large subunit protein of embodiment 32, wherein said modified AGPase large subunit protein is encoded by a polynucleotide that comprises a sequence selected from the group consisting of SEQ ID NOs:13, 47, 59, and 65.
34. The modified AGPase large subunit protein of either one of embodiments 1 and 2 wherein said non-native amino acid residues comprise an asparagine at position 312, glycine at position 317, and arginine at position 599, wherein said positions 312, 317, and 599 correspond to the numbering of SEQ ID NO:3, or wherein said non-native amino acid residues comprise an asparagine at position 158, glycine at position 163, and arginine at position 445, wherein said positions 158, 163, and 445 correspond to the numbering of SEQ ID NO:24.
35. A polynucleotide encoding the modified AGPase large subunit protein of embodiment 34.
36. The modified AGPase large subunit protein of embodiment 4 wherein said AGPase large subunit protein shares at least 80% identity with SEQ ID NO:16 or 54.
37. The modified AGPase large subunit protein of embodiment 36, wherein said modified AGPase large subunit protein is encoded by a polynucleotide that shares at least 70% identity with a sequence selected from the group consisting of SEQ ID NOs:15, 48, 60, and 66.
38. The modified AGPase large subunit protein of embodiment 4 wherein said AGPase large subunit protein comprises SEQ ID NO:16 or 54.
39. The modified AGPase large subunit protein of embodiment 36, wherein said modified AGPase large subunit protein is encoded by a polynucleotide that comprises a sequence selected from the group consisting of SEQ ID NOs:15, 48, 60, and 66.
40. A plant transformation construct comprising a polynucleotide encoding the modified AGPase large subunit of either one of embodiments 1 and 2, wherein said polynucleotide is operably linked to a promoter that is functional in a plant cell.
41. The plant transformation construct of embodiment 40 wherein said polynucleotide encoding the modified AGPase large subunit of embodiment 1 shares at least 70% sequence identity with a sequence selected from the group consisting of SEQ ID NOs:43-48 and 61-66, or encodes a protein that shares at least 80% sequence identity with a sequence selected from the group consisting of SEQ ID NOs:6, 8, 10, 12, 14, 16, and 49-54.
42. The plant transformation construct of embodiment 40 wherein said polynucleotide encoding the modified AGPase large subunit of embodiment 1 is selected from the group consisting of SEQ ID NOs:43-48 and 61-66, or encodes a protein selected from the group consisting of SEQ ID NOs:6, 8, 10, 12, 14, 16, and 49-54.
43. The modified AGPase large subunit protein of either one of embodiments 1 and 2 wherein said modified AGPase large subunit shares at least 80% sequence identity to a sequence selected from the group consisting of SEQ ID NOs:17-42.
44. The plant transformation construct of embodiment 40 wherein said polynucleotide encoding the modified AGPase large subunit of either one of embodiments 1 and 2 encodes a protein with at least 80% identity to a sequence selected from the group consisting of SEQ ID NOs:17-42.
45. A maize plant comprising a modified sh2 gene, said gene having at least 80% identity to a sequence selected from the group consisting of SEQ ID NOs:5, 7, 9, 11, 13, and 15.
46. A maize plant comprising an AGPase large subunit-encoding polynucleotide that encodes the modified AGPase large subunit protein of any one of embodiments 1, 4, 6-10, 12-16, 18-22, 24-28, 30-34, and 36-39.
47. A rice plant comprising a modified AGPase large subunit gene, said gene having at least 80% identity to a sequence selected from the group consisting of SEQ ID NOs:55-60.
48. A rice plant comprising an AGPase large subunit-encoding polynucleotide that encodes the modified AGPase large subunit protein of any one of embodiments 2, 4, 6-10, 12-16, 18-22, 24-28, 30-34, and 36-39.
49. A method of producing the modified AGPase large subunit protein of embodiment 1 in a plant cell comprising generating mutations at the codons encoding the amino acids that correspond to amino acid positions 249, 252, 312, 317 or 599 of SEQ ID NO:3 in the native AGPase large subunit-encoding gene in the genome of one or more plant cells.
50. The method of embodiment 49 wherein said mutations at the codons encoding the amino acids that correspond to amino acid positions 249, 252, 312, 317 or 599 of SEQ ID NO:3 produce an AGPase large subunit gene with at least 80% identity to a sequence selected from the group consisting of SEQ ID NOs:5, 7, 9, 11, 13, and 15.
51. The method of embodiment 49 wherein said mutations at the codons encoding the amino acids corresponding to amino acid positions 249, 252, 312, 317 or 599 of SEQ ID NO:3 produce an AGPase large subunit gene selected from the group consisting of SEQ ID NOs:5, 7, 9, 11, 13, and 15.
52. The method of any one of embodiments 49-51 further comprising regenerating a plant from said one or more plant cells.
53. The plant produced by the method of any one of embodiments 49-52 wherein said one or more plant cells are from Zea mays.
54. A method of producing the modified AGPase large subunit protein of embodiment 2 in a plant cell comprising generating mutations at the codons encoding the amino acids that correspond to amino acid positions 95, 98, 158, 163, or 445 of SEQ ID NO:24 in the native AGPase large subunit-encoding gene in the genome of one or more plant cells.
55. The method of embodiment 54 wherein said mutations at the codons encoding the amino acids that correspond to amino acid positions 95, 98, 158, 163, or 445 of SEQ ID NO:24 produce an AGPase large subunit gene with at least 80% identity to a sequence selected from the group consisting of SEQ ID NOs:55-60.
56. The method of embodiment 49 wherein said mutations at the codons encoding the amino acids corresponding to amino acid positions 95, 98, 158, 163, or 445 of SEQ ID NO:24 produce an AGPase large subunit gene selected from the group consisting of SEQ ID NOs:55-60.
57. The method of any one of embodiments 54-56 further comprising regenerating a plant from said one or more plant cells.
58. The plant produced by the method of any one of embodiments 54-57 wherein said one or more plant cells are from Oryza sativa.
59. A method of producing the modified AGPase large subunit protein of embodiment 1 in a plant cell comprising transforming one or more plant cells with the plant transformation construct of any one of embodiments 40-44.
60. The method of embodiment 59, further comprising regenerating a plant from said one or more plant cells.
61. The method of any one of embodiments 49-52, 54-57, and 59, wherein said plant cells are from a monocot.
62. The method of embodiment 56 wherein said plant cells are from the genus Zea, Oryza, Triticum, Sorghum, Secale, Eleusine, Setaria, Saccharum, Miscanthus, Panicum, Pennisetum, Megathyrsus, Cocos, Ananas, Musa, Elaeis, Avena, or Hordeum.
63. The method of any one of embodiments 49-52, 54-57, and 59, wherein said plant cells are from a dicot.
64. The method of embodiment 58 wherein said plant cells are from the genus Glycine, Brassica, Medicago, Helianthus, Carthamus, Nicotiana, Solanum, Gossypium, Ipomoea, Manihot, Coffea, Citrus, Theobroma, Camellia, Persea, Ficus, Psidium, Mangifera, Olea, Carica, Anacardium, Macadamia, Prunus, Beta, Populus, or Eucalyptus.
65. Seed of a plant comprising a polynucleotide of any one of embodiments 3, 5, 11, 17, 23, 29, and 35.
66. Seed of a plant comprising a polynucleotide encoding the modified AGPase large subunit protein of any one of embodiments 1, 2, 4, 6-10, 12-16, 18-22, 24-28, 30-34, and 36-39.
67. A repair donor template molecule comprising a desired genome editing mutation and one or more primer pads, wherein said primer pads comprise at least five primer pad mutations as compared to the targeted wild-type DNA sequence.
68. The repair donor template molecule of embodiment 67, wherein said primer pads comprise at least ten primer pad mutations as compared to the targeted wild-type DNA sequence.
69. The repair donor template molecule of embodiment 67, wherein said primer pads comprise at least fifteen primer pad mutations as compared to the targeted wild-type DNA sequence.
70. The repair donor template molecule of embodiment 67, wherein said repair donor template molecule comprises single-stranded DNA (ssDNA).
71. The repair donor template molecule of embodiment 67, wherein said repair donor template molecule comprises double-stranded DNA (dsDNA).
72. The repair donor template molecule of embodiment 67, wherein said repair donor template molecule comprises circular DNA.
73. The repair donor template molecule of embodiment 67, wherein said repair donor template molecule comprises two or more primer pads.
74. The repair donor template molecule of embodiment 67, wherein said primer pad mutations comprise silent mutations in a coding region.
75. The repair donor template molecule of embodiment 67, wherein said primer pad mutations are located in a non-coding region of DNA.
76. The repair donor template molecule of embodiment 67, wherein said one or more primer pads surround said desired genome editing mutation.
77. The repair donor template molecule of embodiment 67, wherein said one or more primer pads are located upstream of said desired genome editing mutation.
78. The repair donor template molecule of embodiment 67, wherein said one or more primer pads are located downstream of said desired genome editing mutation.
79. The repair donor template molecule of embodiment 76 further comprising at least a second primer pad located upstream of said desired genome editing mutation.
80. The repair donor template molecule of embodiment 76 further comprising at least a second primer pad located downstream of said desired genome editing mutation.
81. The repair donor template molecule of embodiment 77 further comprising at least a second primer pad located downstream of said desired genome editing mutation.
82. A method of detecting homology-directed repair (HDR) mutation events comprising performing a polymerase chain reaction (PCR) using DNA extracted from one or more cells that have been exposed to a repair donor template comprising a desired genome editing mutation and at least one primer pad, wherein said PCR uses at least one primer designed to anneal to said primer pad, wherein said primer designed to anneal to said primer pad does not effectively anneal to the wild-type sequence.
83. A method of selecting cells that comprise a desired genomic modification comprising
   a. detecting the presence of said desired genome editing mutation using the method of embodiment 82, and
   b. allowing cells comprising said desired genome editing mutation to multiply.
84. The method of embodiment 83, further comprising allowing cells comprising said desired genome editing mutation to regenerate into a tissue or an organism.
85. The repair donor template of embodiment 67, wherein said repair donor template comprises SEQ ID NO:74.
86. The repair donor template of embodiment 67, wherein said repair donor template comprises nucleotides 235-252 or nucleotides 781-798 of SEQ ID NO:74.
87. The method of embodiment 82, wherein said one or more cells that has been exposed to a repair donor template is/are prokaryotic cells.
88. The method of embodiment 82, wherein said one or more cells that has been exposed to a repair donor template is/are eukaryotic cells.

### BRIEF DESCRIPTION OF THE DRAWINGS

FIG. 1 shows a MUSCLE alignment of AGPase large subunit proteins from the maize sh2 gene (SEQ ID NOs:1 and 2, encoding SEQ ID NO:3) and the potato AGPase large subunit (SEQ ID NO:4).

FIG. 2 shows a MUSCLE alignment of AGPase large subunit proteins (SEQ ID NOs:3 and 17-42). Conserved residues are indicated by an asterisk (*), while colons (:) and periods (.) indicate conservative substitutions at that site. Arrows indicate the amino acid residues corresponding to the T249, Q252, G312, D317, and A599 amino acids in the maize sh2 protein (SEQ ID NO:3) and to T95, Q98, G158, D163, and T445 in the rice AGPase large subunit protein (SEQ ID NO:24).

FIG. 3 shows a schematic depiction of primer pad locations. Four possible locations of one or more primer pads are depicted. The size and location of the repair donor template is shown as a gray bar above each of the four depicted schematics, and the intended base change is depicted as a vertical line. Primer pads are shown as gray boxes and primers are shown as small arrows below each of the schematic diagrams.

### DETAILED DESCRIPTION OF THE INVENTION

Compositions and methods for increasing crop biomass and yield are provided. In some embodiments, the methods of the invention and plants produced thereby increase crop biomass and yield under conditions of heat and/or drought stress. The methods include expressing at least one AGPase large subunit gene in a plant of interest. The AGPase large subunit coding sequence may be modified in a plant of interest. Alternatively, the plant of interest can be transformed with at least one modified AGPase large subunit gene. Plants, seeds, and plant parts expressing the modified AGPase large subunit protein and have increased yield are encompassed by the invention.

Crop yield is an extremely complex trait that results from the growth of a crop plant through all stages of its development and allocation of plant resources to the harvestable portions of the plant. In some crops including but not limited to maize and soybean, the primary harvestable portions may include seeds, with secondary applications from the remainder of the biomass (e.g., leaves and stems). In other crops, including but not limited to sugarcane and alfalfa, the primary harvestable portions of the plant consist of the stems or entire above-ground portion of the plant. In other crops including, but not limited to potato and carrot, the primary harvestable portions of the plant are found below-ground. Regardless of the harvested portion(s) of the crop plant, the accumulation of harvestable biomass results from plant growth and allocation of photosynthetically fixed carbon to the harvested portion(s) of the plant. Plant growth may be manipulated by modulating the expression of one or more plant genes, i.e., an AGPase large subunit. This modulation can alter the function of one or more metabolic pathways that contributes to plant growth and accumulation of harvestable biomass.

Methods of the invention include the modification of the native coding sequence of one or more genes encoding an AGPase large subunit protein. In some embodiments, the AGPase large subunit protein-encoding sequence is found in a plant genome. The AGPase large subunit protein encoded by the modified AGPase large subunit coding sequence provides for improved enzyme kinetics and/or improved thermotolerance of the AGPase holoenzyme relative to the unmodified enzyme. In some embodiments, expression of the modified AGPase large subunit coding sequence results in increased harvestable biomass in plants comprising the modified AGPase large subunit coding sequence relative to control plants. Any method that provides for expression of a modified AGPase large subunit in a plant of interest is encompassed by the present invention. For example, the AGPase large subunit coding sequence may be modified in the plant of interest. In other embodiments, the target plant may be transformed to express a modified AGPase large subunit coding sequence expressing the modified protein.

The compositions of the invention include the polynucleotide sequences set forth in SEQ ID NOs:5, 7, 9, 11, 13, 15, 43-48, and 55-66 or a polynucleotide encoding a protein selected from the group consisting of SEQ ID NOs:6, 8, 10, 12, 14, 16, and 49-54 or variants thereof. In some embodiments, the polynucleotide encodes an AGPase large subunit protein comprising a lysine at position 249, glycine at position 252, asparagine at position 312, glycine at position 317, and/or arginine at position 599 according to the numbering of the maize sh2 protein (SEQ ID NO:3). In other embodiments, the polynucleotide encodes an AGPase large subunit protein comprising a lysine at position 95, glycine at position 98, asparagine at position 158, glycine at position 163, and/or arginine at position 445 according to the numbering of the rice AGPase large subunit protein (SEQ ID NO:24). It is recognized that having identified the desirable modified AGPase large subunit coding sequences and encoded protein sequences disclosed herein, it is within the state of the art to identify and/or design additional modified AGPase large subunit protein sequences and nucleotide sequences encoding said modified AGPase large subunit protein sequences, for instance through BLAST searches, PCR assays, and the like.

Methods and compositions are provided herein for the precise modification of genomic DNA sequences, such as genome perturbation or gene-editing, that relate to the use of a repair donor template that comprises mutations relative to unmodified (or wild-type) DNA. These mutations may be used for detection of the resulting modified DNA in cells that have been treated using the methods of the invention, for example through the use of PCR-based assays. Following a genome editing experiment, typically only a proportion of the treated cells will comprise the desired site-specific mutation(s). Identification of those cells that comprise the desired mutation(s) may be difficult using standard methods, particularly in cases where only one or a small number of base changes result from genome editing. The present invention provides methods and compositions to overcome these difficulties.

In some cases, genome editing experiments may seek to modify only one or a few bases of DNA relative to the native sequence. This may be, for example, because a particular base change is associated with disease or susceptibility to disease, because a particular base change results in destruction or creation of an open-reading frame, because a particular base change is associated with a more desirable phenotype, or for another reason. Repair donor templates for those cases may comprise only one mutation or only a few mutations relative to the native or wild-type sequence. In those cases, detection of the desired genome editing events using standard screening methods such as the T7 endonuclease or SURVEYOR endonuclease may be difficult. PCR primers designed to anneal to the modified site will very likely bind to both the native and to the modified sequence because of the small number of sequence changes between the two. As a result, laborious and time-consuming DNA sequencing methods may be required to identify cells that may comprise the desired genome edits. The present invention provides repair donor templates that comprise “primer pads,” or sequences that differ substantially from the native sequence to allow primers to bind to the modified sequence far more efficiently than to the native sequence. Primer pads serve only as a marker for gene editing within a particular cell, which is in contrast to the desired or intended genome edit(s) that are introduced in order to alter the expression of a gene or the resultant gene product, and in some cases the phenotype of the cell. For this reason, primer pads may be designed in non-coding regions of the genome (e.g., introns, intergenic regions, or other non-coding regions of DNA) or may be designed in coding regions through the use of silent mutations to retain the encoded amino acid sequence. The primer pads may surround the intended genome edit(s) or may be closely genetically linked to the intended edit(s). By designing primers to anneal to the primer pads, it is possible to very readily detect likely cells or groups of cells that comprise the desired genome edit(s) using rapid and straightforward PCR assays. After PCR assays are used to identify those cells or molecules that comprise the desired mutation(s), those cells may be cultured, and in some embodiments may be regenerated into tissues or organisms comprising DNA that comprises the desired mutations.

In some embodiments, a primer pad comprises at least 5, 6, 7, 8, 9, 10, 11, 12, 13, 14, 15, 16, 17, 18, 19, 20, 21, 22, 23, 24, 25 or more than 25 mutations, herein referred to a primer pad mutations, relative to the unmodified or wild-type DNA in addition to the intended or desired genome edit(s). In some embodiments, the primer pad(s) comprise(s) silent mutations within a coding region, resulting in the production of a polypeptide with an identical amino acid sequence as compared with the unmodified or wild-type polypeptide. In some embodiments, the primer pad(s) may comprise mutations in a non-coding region such as an intron.

In some embodiments, the primer pad(s) may surround one or more intended genome edits, (e.g., non-silent mutations). In some embodiments the primer pad(s) may be located upstream and/or downstream of one or more intended genome edits (e.g., non-silent mutations). In those embodiments where the primer pad(s) is/are located upstream and/or downstream of the one or more intended genome edits (e.g., non-silent mutations), the primer pad(s) may be separated from the intended genome edits (e.g., non-silent mutations) by 1 or more, 5 or more, 10 or more, 15 or more, 20 or more, or 25 or more base pairs. In preferred embodiments, the primer pad(s) are separated from the intended genome edits (e.g., non-silent mutations) by fewer than 40 base pairs.

While the method of detection of modified DNA is exemplified herein using an AGPase sequence, it is recognized that any modification can be detected using the primer pads of the invention. That is, the primer pad can be used as a signature to detect any modification. Such modification can be made in any cell type, prokaryotic or eukaryotic and with any editing technology. Such technology includes CRISPR nucleases such as Cas9, Cpf1, Cms1, CasX, CasY, C2c1, C2c3, or other suitable CRISPR nucleases, as well as with site-specific nucleases including but not limited to meganucleases, zinc-finger nucleases (ZFNs), TALENs, homing endonucleases, and other nucleases capable of producing single-stranded or double-stranded breaks at a predetermined site or sites. In each case, the site-specific nuclease or site-specific nucleases is/are used to produce one or more single-stranded breaks or, in a preferred embodiment, one or more double-stranded breaks at a pre-determined site or sites in targeted DNA. The DNA is then repaired, resulting in the incorporation of a repair donor template comprising the primer pads of the present invention into the targeted DNA at or near the site of the single-stranded and/or double-stranded breaks in the targeted DNA. The incorporated repair donor template sequence may then be detected using the methods of the invention as described herein.

The coding sequences of the present invention, when operably linked to a promoter that is functional in a plant cell, enable expression and accumulation of modified AGPase large subunit protein in the cells of a plant comprising the modified AGPase large subunit cassette. “Operably linked” is intended to mean a functional linkage between two or more elements. For example, an operable linkage between a promoter and a nucleotide of interest is a functional link that allows for expression of the nucleotide sequence of interest. Operably linked elements may be contiguous or non-contiguous. When used to refer to the joining of two protein coding regions, by operably linked is intended that the coding regions are in the same reading frame. The cassette may additionally contain at least one additional gene to be co-transformed into the plant. Alternatively, the additional gene(s) can be provided on multiple expression cassettes or DNA constructs. The expression cassette may additionally contain selectable marker genes. “Modified AGPase large subunit cassette” is intended to mean a modified AGPase large subunit coding sequence of the present invention, operably linked to a functional promoter. In some embodiments, the nucleotide sequences encoding the modified AGPase large subunit proteins are modified in the plant and therefore may be expressed by its native promoter.

In some embodiments, the nucleotide sequences encoding the modified AGPase large subunit proteins of the invention are found in a plant genome, operably linked to one or more promoters that are functional in a plant cell for expression in the plant cell or plant of interest. In some embodiments, said promoter that is functional in a plant cell is preferentially expressed in endosperm tissue. In other embodiments, said promoter that is functional in a plant cell is preferentially expressed in leaf tissue, in bundle sheath cells, or is constitutively expressed. By “preferentially expressed” is intended to mean that expression, e.g., in a particular cell type, a particular tissue type, or at a particular time during the circadian cycle is higher than in a different cell type, tissue type, or at a different time during the circadian cycle, as appropriate. Expression levels may be determined using standard molecular assays including RT-PCR, Northern blotting, and the like. It is recognized that the promoter that is operably linked to the modified AGPase large subunit protein-encoding sequence of the invention may be naturally operably linked to an AGPase large subunit coding sequence in its native context, or may be heterologous to an AGPase large subunit coding sequence.

Fragments and variants of the polynucleotides and amino acid sequences of the present invention may also be expressed by promoters that are operable in plant cells. By “fragment” is intended a portion of the polynucleotide or a portion of the amino acid sequence. “Variants” is intended to mean substantially similar sequences. For polynucleotides, a variant comprises a polynucleotide having deletions (i.e., truncations) at the 5′ and/or 3′ end; deletion and/or addition of one or more nucleotides at one or more internal sites in the native polynucleotide; and/or substitution of one or more nucleotides at one or more sites in the native polynucleotide. As used herein, a “native” polynucleotide or polypeptide comprises a naturally occurring nucleotide sequence or amino acid sequence, respectively. Generally, variants of a particular polynucleotide of the invention will have at least about 75%, 80%, 85%, 90%, 91%, 92%, 93%, 94%, 95%, 96%, 97%, 98%, 99% or more sequence identity to that particular polynucleotide as determined by sequence alignment programs and parameters as described elsewhere herein. Fragments and variants of the polynucleotides disclosed herein can encode proteins that retain AGPase large subunit function.

“Variant” amino acid or protein is intended to mean an amino acid or protein derived from the native amino acid or protein by deletion (so-called truncation) of one or more amino acids at the N-terminal and/or C-terminal end of the native protein; deletion and/or addition of one or more amino acids at one or more internal sites in the native protein; or substitution of one or more amino acids at one or more sites in the native protein. Variant proteins encompassed by the present invention are biologically active, that is they continue to possess the desired biological activity of the native protein, such as conversion of glucose-1-phosphate to ADP-glucose when incorporated into an AGPase holoenzyme that in some embodiments comprises two AGPase large subunit and two AGPase small subunit proteins. Biologically active variants of a native polypeptide will have at least about 80%, 85%, 90%, 91%, 92%, 93%, 94%, 95%, 96%, 97%, 98%, 99% or more sequence identity to the amino acid sequence for the native sequence as determined by sequence alignment programs and parameters described herein. In some embodiments, the variant polypeptide sequences will comprise conservative amino acid substitutions. The number of such conservative amino acid substitutions, summed with the number of amino acid identities, can be used to calculate the sequence positives when this sum is divided by the total number of amino acids in the sequence of interest. Sequence positive calculations are performed on the NCBI BLAST server that can be accessed on the world wide web at blast.ncbi.nlm.nih.gov/Blast.cgi. A biologically active variant of a protein of the invention may differ from that protein by as few as 1-15 amino acid residues, as few as 1-10, such as 6-10, as few as 5, as few as 4, 3, 2, or even 1 amino acid residue.

Amino acids can be generally categorized as aliphatic, hydroxyl or sulfur/selenium-containing, cyclic, aromatic, basic, or acidic and their amide. Without being limited by theory, conservative amino acid substitutions may be preferable in some cases to non-conservative amino acid substitutions for the generation of variant protein sequences, as conservative substitutions may be more likely than non-conservative substitutions to allow the variant protein to retain its biological activity. Polynucleotides encoding a polypeptide having one or more amino acid substitutions in the sequence are contemplated within the scope of the present invention. Table 1 below provides a listing of examples of amino acids belong to each class.

TABLE 1
Classes of Amino Acids

| Amino Acid Class | Example Amino Acids |
|---|---|
| Aliphatic | Gly, Ala, Val, Leu, Ile |
| Hydroxyl or | Ser, Cys, Thr, Met, Sec |
| sulfur/selenium- | |
| containing | |
| Cyclic | Pro |
| Aromatic | Phe, Tyr, Trp |
| Basic | His, Lys, Arg |
| Acidic and their | Asp, Glu, Asn, Gln |
| Amide | |

Variant sequences may also be identified by analysis of existing databases of sequenced genomes. In this manner, corresponding sequences can be identified and used in the methods of the invention.

Methods of alignment of sequences for comparison are well known in the art. Thus, the determination of percent sequence identity between any two sequences can be accomplished using a mathematical algorithm. Non-limiting examples of such mathematical algorithms are the algorithm of Myers and Miller (1988) CABIOS 4:11-17; the local alignment algorithm of Smith et al. (1981) Adv. Appl. Math. 2:482; the global alignment algorithm of Needleman and Wunsch (1970) J. Mol. Biol. 48:443-453; the search-for-local alignment method of Pearson and Lipman (1988) Proc. Nat. Acad. Sci. 85:2444-2448; the algorithm of Karlin and Altschul (1990) Proc. Nat. Acad. Sci. USA 87:2264-2268, modified as in Karlin and Altschul (1993) Proc. Nat. Acad. Sci. USA 90:5873-5877.

Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA, and TFASTA in the GCG Wisconsin Genetics Software Package, Version 10 (available from Accelrys Inc., 9685 Scranton Road, San Diego, Calif., USA). Alignments using these programs can be performed using the default parameters. The CLUSTAL program is well described by Higgins et al. (1988) Gene 73:237-244 (1988); Higgins et al. (1989) CABIOS 5:151-153; Corpet et al. (1988) Nucleic Acids Res. 16:10881-90; Huang et al. (1992) CABIOS 8:155-65; and Pearson et al. (1994) Meth. Mol. Biol. 24:307-331. The ALIGN program is based on the algorithm of Myers and Miller (1988) supra. A PAM120 weight residue table, a gap length penalty of 12, and a gap penalty of 4 can be used with the ALIGN program when comparing amino acid sequences. The BLAST programs of Altschul et al (1990) J. Mol. Biol. 215:403 are based on the algorithm of Karlin and Altschul (1990) supra. BLAST nucleotide searches can be performed with the BLASTN program, score=100, wordlength=12, to obtain nucleotide sequences homologous to a nucleotide sequence encoding a protein of the invention. BLAST protein searches can be performed with the BLASTX program, score=50, wordlength=3, to obtain amino acid sequences homologous to a protein or polypeptide of the invention. To obtain gapped alignments for comparison purposes, Gapped BLAST (in BLAST 2.0) can be utilized as described in Altschul et al. (1997) Nucleic Acids Res. 25:3389. Alternatively, PSI-BLAST (in BLAST 2.0) can be used to perform an iterated search that detects distant relationships between molecules. See Altschul et al. (1997) supra. When utilizing BLAST, Gapped BLAST, PSI-BLAST, the default parameters of the respective programs (e.g., BLASTN for nucleotide sequences, BLASTX for proteins) can be used. See www.ncbi.nlm.nih.gov. Alignment may also be performed manually by inspection.

Such genes and coding regions can be codon optimized for expression in a plant of interest. A “codon-optimized gene” is a gene having its frequency of codon usage designed to mimic the frequency of preferred codon usage of the host cell. Nucleic acid molecules can be codon optimized, either wholly or in part. Because any one amino acid (except for methionine and tryptophan) is encoded by a number of codons, the sequence of the nucleic acid molecule may be changed without changing the encoded amino acid. Codon optimization is when one or more codons are altered at the nucleic acid level such that the amino acids are not changed but expression in a particular host organism is increased. Those having ordinary skill in the art will recognize that codon tables and other references providing preference information for a wide range of organisms are available in the art (see, e.g., Zhang et al. (1991) Gene 105:61-72; Murray et al. (1989) Nucl. Acids Res. 17:477-508). Methodology for optimizing a nucleotide sequence for expression in a plant is provided, for example, in U.S. Pat. No. 6,015,891, and the references cited therein, as well as in WO 2012/142,371, and the references cited therein.

The nucleotide sequences of the invention may be used in recombinant polynucleotides. A “recombinant polynucleotide” comprises a combination of two or more chemically linked nucleic acid segments which are not found directly joined in nature. By “directly joined” is intended the two nucleic acid segments are immediately adjacent and joined to one another by a chemical linkage. In specific embodiments, the recombinant polynucleotide comprises a polynucleotide of interest or active variant or fragment thereof such that an additional chemically linked nucleic acid segment is located either 5′, 3′ or internal to the polynucleotide of interest. Alternatively, the chemically-linked nucleic acid segment of the recombinant polynucleotide can be formed by deletion of a sequence. The additional chemically linked nucleic acid segment or the sequence deleted to join the linked nucleic acid segments can be of any length, including for example, 1, 2, 3, 4, 5, 6, 7, 8, 9, 10, 15, 20 or greater nucleotides. Various methods for making such recombinant polynucleotides are disclosed herein, including, for example, by chemical synthesis or by the manipulation of isolated segments of polynucleotides by genetic engineering techniques. In specific embodiments, the recombinant polynucleotide can comprise a recombinant DNA sequence or a recombinant RNA sequence. A “fragment of a recombinant polynucleotide” comprises at least one of a combination of two or more chemically linked amino acid segments which are not found directly joined in nature.

By “altering” or “modulating” the expression level of a gene is intended that the expression of the gene is upregulated or downregulated. It is recognized that in some instances, plant growth and yield are increased by increasing the expression levels of one or more genes encoding AGPase large subunit proteins, i.e. upregulating expression. Likewise, in some instances, plant growth and yield may be increased by decreasing the expression levels of one or more genes encoding AGPase large subunit proteins, i.e. downregulating expression. Thus, the invention encompasses the upregulation or downregulation of one or more genes encoding AGPase large subunit proteins. Further, the methods include the upregulation of at least one gene encoding an AGPase large subunit protein and the downregulation of at least one gene encoding a second AGPase large subunit protein in a plant of interest. By modulating the concentration and/or activity of at least one of the genes encoding an AGPase large subunit protein in a plant is intended that the concentration and/or activity is increased or decreased by at least about 1%, about 5%, about 10%, about 20%, about 30%, about 40%, about 50%, about 60%, about 70%, about 80%, or about 90% or greater relative to a control plant, plant part, or cell which did not have the sequence of the invention introduced.

By “modifying” a coding sequence is intended that one or more codons is substituted with a codon encoding a non-synonymous amino acid, has one or more codons inserted into the coding sequence, and/or has one or more codons removed from the coding sequence, such that the encoded protein has one or more amino acid changes, insertions, and/or deletions relative to the unmodified or native protein.

As indicated above, the expression levels of the genes encoding the modified AGPase large subunit proteins of the present invention can be controlled by the use of one or more promoters that are functional in a plant cell. The expression level of the modified AGPase large subunit protein-encoding gene of interest may be measured directly, for example, by assaying for the level of the AGPase large subunit gene transcript or of the encoded protein in the plant. Methods for such assays are well-known in the art. For example, Northern blotting or quantitative reverse transcriptase-PCR (qRT-PCR) may be used to assess transcript levels, while western blotting, ELISA assays, or enzyme assays may be used to assess protein levels. AGPase large subunit function can be assessed by, for example, monitoring the formation of ADP-glucose from glucose-1-phosphate.

A “subject plant or plant cell” is one comprising a modified AGPase large subunit coding sequence or is a plant or plant cell which is descended from a plant or cell so altered and which comprises the alteration. As discussed, the AGPase large subunit sequence may be introduced into the plant by transformation or by directly altering the native AGPase large subunit coding sequence. A “control” or “control plant” or “control plant cell” provides a reference point for measuring changes in phenotype of the subject plant or plant cell. Thus, the expression levels of a modified AGPase large subunit protein-encoding gene of interest may be higher, lower, or the same as the expression levels of an unmodified AGPase large subunit protein-encoding gene in the control plant depending on the methods of the invention. Similarly, the cell type, tissue type, circadian cycle, and other expression patterns for the modified AGPase large subunit protein-encoding genes may be the same as, or different from, the expression patterns for unmodified AGPase large subunit protein-encoding genes in control plants or control plant cells.

A control plant or plant cell may comprise, for example: (a) a wild-type plant or cell, i.e., of the same genotype as the starting material for the genetic alteration which resulted in the subject plant or cell; (b) a plant or plant cell of the same genotype as the starting material but which has been transformed with a null construct (i.e. with a construct which has no known effect on the trait of interest, such as a construct comprising a marker gene); (c) a plant or plant cell which is a non-transformed segregant among progeny of a subject plant or plant cell; (d) a plant or plant cell genetically identical to the subject plant or plant cell but which is not exposed to conditions or stimuli that would induce expression of the gene of interest; or (e) the subject plant or plant cell itself, under conditions in which the gene of interest is not expressed.

While the invention may be described in terms of transformed or altered plants, it is recognized that transformed or altered organisms of the invention also include plant cells, plant protoplasts, plant cell tissue cultures from which plants can be regenerated, plant calli, plant clumps, and plant cells that are intact in plants or parts of plants such as embryos, pollen, ovules, seeds, leaves, flowers, branches, fruit, kernels, ears, cobs, husks, stalks, roots, root tips, anthers, and the like. Grain is intended to mean the mature seed produced by commercial growers for purposes other than growing or reproducing the species. Progeny, variants, and mutants of the regenerated plants are also included within the scope of the invention, provided that these parts comprise the modified polynucleotides.

To downregulate expression of an AGPase large subunit protein-encoding gene of interest, antisense constructions, complementary to at least a portion of the messenger RNA (mRNA) for the sequences of a gene of interest, particularly a gene encoding an AGPase large subunit protein of interest can be constructed. Antisense nucleotides are designed to hybridize with the corresponding mRNA. Modifications of the antisense sequences may be made as long as the sequences hybridize to and interfere with expression of the corresponding mRNA. In this manner, antisense constructions having 70%, optimally 80%, more optimally 85%, 90%, 95% or greater sequence identity to the corresponding sequences to be silenced may be used. Furthermore, portions of the antisense nucleotides may be used to disrupt the expression of the target gene. Alternatively, downregulation of an AGPase large subunit protein-encoding gene of interest may be accomplished by site-specific modification of sequences that regulate expression of said gene; nucleotides may for instance be inserted or deleted from promoters or other regulatory elements, or the sequence of one or more regulatory elements may be modified such that expression of an AGPase large subunit protein-encoding gene is decreased relative to a control plant or plant cell. In some embodiments, expression of an AGPase large subunit protein-encoding gene may be abolished through site-specific modifications to the coding sequence or to regulatory elements that regulate expression of an AGPase large subunit protein-encoding gene. For instance, a promoter may be deleted entirely, or portions of the promoter that are required for its function may be deleted. Alternatively, the start codon for the coding sequence may be deleted or modified such that a functional open-reading frame is no longer present, resulting in the abolishment of AGPase large subunit protein accumulation.

The polynucleotides of the invention can be used to isolate corresponding sequences from other plants. In this manner, methods such as PCR, hybridization, and the like can be used to identify such sequences based on their sequence homology or identity to the sequences set forth herein. Sequences isolated based on their sequence identity to the entire sequences set forth herein or to variants and fragments thereof are encompassed by the present invention. Such sequences include sequences that are orthologs of the disclosed sequences. “Orthologs” is intended to mean genes derived from a common ancestral gene and which are found in different species as a result of speciation. Genes found in different species are considered orthologs when their nucleotide sequences and/or their encoded protein sequences share at least 75%, 80%, 85%, 90%, 91%, 92%, 93%, 94%, 95%, 96%, 97%, 98%, 99%, or greater sequence identity. Functions of orthologs are often highly conserved among species. Thus, isolated polynucleotides that have transcription activation or enhancer activities and which share at least 75% sequence identity to the sequences disclosed herein, or to variants or fragments thereof, are encompassed by the present invention.

Variant or homologous sequences can be isolated from other organisms, for instance by PCR. Methods for designing PCR primers and PCR cloning are generally known in the art and are disclosed in Sambrook et al. (1989) Molecular Cloning: A Laboratory Manual (2d ed., Cold Spring Harbor Laboratory Press, Plainview, N.Y.). See also Innis et al., eds. (1990) PCR Protocols: A Guide to Methods and Applications (Academic Press, New York); Innis and Gelfand, eds. (1995) PCR Strategies (Academic Press, New York); and Innis and Gelfand, eds. (1999) PCR Methods Manual (Academic Press, New York).

Variant sequences may also be identified by analysis of existing databases of sequenced genomes. In this manner, corresponding sequences encoding AGPase large subunit proteins can be identified and used in the methods of the invention. The variant sequences will retain the biological activity of an AGPase large subunit protein (i.e., conversion of glucose-1-phosphate to ADP-glucose when present as part of an AGPase holoenzyme). The present invention provides for modified AGPase large subunit proteins and encoding polynucleotides that, when expressed in the proper manner in a plant, can lead to increased biomass and seed yield.

The expression cassette will include in the 5′-3′ direction of transcription, a transcriptional and translational initiation region, a polynucleotide encoding a modified AGPase large subunit protein of the present invention, and a transcriptional and translational termination region (i.e., termination region) functional in plants.

A number of promoters may be used in the practice of the invention. The polynucleotides encoding a modified AGPase large subunit protein of the invention may be expressed from a promoter with a constitutive expression profile. Constitutive promoters include the CaMV 35S promoter (Odell et al. (1985) Nature 313:810-812); rice actin (McElroy et al. (1990) Plant Cell 2:163-171); ubiquitin (Christensen et al. (1989) Plant Mol. Biol. 12:619-632 and Christensen et al. (1992) Plant Mol. Biol. 18:675-689); pEMU (Last et al. (1991) Theor. Appl. Genet. 81:581-588); MAS (Velten et al. (1984) EMBO J. 3:2723-2730); ALS promoter (U.S. Pat. No. 5,659,026), and the like.

Polynucleotides of the invention encoding modified AGPase large subunit proteins of the invention may be expressed from tissue-preferred promoters. Tissue-preferred promoters include Yamamoto et al. (1997) Plant J. 12(2):255-265; Kawamata et al. (1997) Plant Cell Physiol. 38(7):792-803; Hansen et al. (1997) Mol. Gen Genet. 254(3):337-343; Russell et al. (1997) Transgenic Res. 6(2):157-168; Rinehart et al. (1996) Plant Physiol. 112(3):1331-1341; Van Camp et al. (1996) Plant Physiol. 112(2):525-535; Canevascini et al. (1996) Plant Physiol. 112(2):513-524; Yamamoto et al. (1994) Plant Cell Physiol. 35(5):773-778; Lam (1994) Results Probl. Cell Differ. 20:181-196; Orozco et al. (1993) Plant Mol Biol. 23(6):1129-1138; Matsuoka et al. (1993) Proc Natl. Acad. Sci. USA 90(20):9586-9590; and Guevara-Garcia et al. (1993) Plant J. 4(3):495-505. Leaf-preferred promoters are also known in the art. See, for example, Yamamoto et al. (1997) Plant J. 12(2):255-265; Kwon et al. (1994) Plant Physiol. 105:357-67; Yamamoto et al. (1994) Plant Cell Physiol. 35(5):773-778; Gotor et al. (1993) Plant J. 3:509-18; Orozco et al. (1993) Plant Mol. Biol. 23(6):1129-1138; and Matsuoka et al. (1993) Proc. Natl. Acad. Sci. USA 90(20):9586-9590.

Developmentally-regulated promoters may be desirable for the expression of a polynucleotide encoding a modified AGPase large subunit protein. Such promoters may show a peak in expression at a particular developmental stage. Such promoters have been described in the art, e.g., U.S. 62/029,068; Gan and Amasino (1995) Science 270: 1986-1988; Rinehart et al. (1996) Plant Physiol 112: 1331-1341; Gray-Mitsumune et al. (1999) Plant Mol Biol 39: 657-669; Beaudoin and Rothstein (1997) Plant Mol Biol 33: 835-846; Genschik et al. (1994) Gene 148: 195-202, and the like.

Promoters that are induced following the application of a particular biotic and/or abiotic stress may be desirable for the expression of a polynucleotide encoding a modified AGPase large subunit protein. Such promoters have been described in the art, e.g., Yi et al. (2010) Planta 232: 743-754; Yamaguchi-Shinozaki and Shinozaki (1993)Mol Gen Genet 236: 331-340; U.S. Pat. No. 7,674,952; Rerksiri et al. (2013) Sci World J 2013: Article ID 397401; Khurana et al. (2013) PLoS One 8: e54418; Tao et al. (2015) Plant Mol Biol Rep 33: 200-208, and the like.

Cell-preferred promoters may be desirable for the expression of a polynucleotide encoding a modified AGPase large subunit protein. Such promoters may preferentially drive the expression of a downstream gene in a particular cell type such as a mesophyll or a bundle sheath cell. Such cell-preferred promoters have been described in the art, e.g., Viret et al. (1994) Proc Natl Acad USA 91: 8577-8581; U.S. Pat. Nos. 8,455,718; 7,642,347; Sattarzadeh et al. (2010) Plant Biotechnol J 8: 112-125; Engelmann et al. (2008) Plant Physiol 146: 1773-1785; Matsuoka et al. (1994) Plant J 6: 311-319, and the like.

The genes encoding modified AGPase large subunit proteins of the invention may be heterologous to the plant in which they are expressed. In such embodiments, the gene encoding a modified AGPase large subunit protein may be functionally linked to a promoter that regulates the expression of an AGPase large subunit in its native context, or may be functionally linked to a promoter that does not regulate the expression of an AGPase large subunit in its native context. In other embodiments, the genes encoding modified AGPase large subunit proteins of the invention will be native to the plant in which they are expressed, but will comprise one or more non-naturally occurring mutations in the coding sequence, e.g., as a result of site-specific genomic modifications. In certain embodiments, modified AGPase large subunit protein-encoding genes that comprise one or more non-naturally occurring mutations will be located at the native locus for the AGPase large subunit in the plant genome of interest and as such will be expressed from the native AGPase large subunit promoter with a similar or identical expression level and profile. In other embodiments, a non-native promoter will be used to regulate the expression of the modified AGPase large subunit protein-encoding gene.

It is recognized that a specific, non-constitutive expression profile may provide an improved plant phenotype relative to constitutive expression of a gene or genes of interest. For instance, many plant genes are regulated by light conditions, the application of particular stresses, the circadian cycle, or the stage of a plant's development. These expression profiles may be important for the function of the gene or gene product in planta. One strategy that may be used to provide a desired expression profile is the use of synthetic promoters containing cis-regulatory elements that drive the desired expression levels at the desired time and place in the plant. Cis-regulatory elements that can be used to alter gene expression in planta have been described in the scientific literature (Vandepoele et al. (2009) Plant Physiol 150: 535-546; Rushton et al. (2002) Plant Cell 14: 749-762). Cis-regulatory elements may also be used to alter promoter expression profiles, as described in Venter (2007) Trends Plant Sci 12: 118-124.

Plant terminators are known in the art and include those available from the Ti-plasmid of A. tumefaciens, such as the octopine synthase and nopaline synthase termination regions. See also Guerineau et al. (1991) Mol. Gen. Genet. 262:141-144; Proudfoot (1991) Cell 64:671-674; Sanfacon et al. (1991) Genes Dev. 5:141-149; Mogen et al. (1990) Plant Cell 2:1261-1272; Munroe et al. (1990) Gene 91:151-158; Ballas et al. (1989) Nucleic Acids Res. 17:7891-7903; and Joshi et al. (1987) Nucleic Acids Res. 15:9627-9639.

In some embodiments, the nucleotides encoding modified AGPase large subunit proteins of the present invention can be used in expression cassettes to transform plants of interest. Transformation protocols as well as protocols for introducing polypeptides or polynucleotide sequences into plants may vary depending on the type of plant or plant cell, i.e., monocot or dicot, targeted for transformation. The term “transform” or “transformation” refers to any method used to introduce polypeptides or polynucleotides into plant cells. Suitable methods of introducing polypeptides and polynucleotides into plant cells include microinjection (Crossway et al. (1986) Biotechniques 4:320-334), electroporation (Riggs et al. (1986) Proc. Natl. Acad. Sci. USA 83:5602-5606, Agrobacterium-mediated transformation (U.S. Pat. Nos. 5,563,055 and 5,981,840), direct gene transfer (Paszkowski et al. (1984) EMBO J. 3:2717-2722), and ballistic particle acceleration (see, for example, U.S. Pat. Nos. 4,945,050; 5,879,918; 5,886,244; and, 5,932,782; Tomes et al. (1995) in Plant Cell, Tissue, and Organ Culture: Fundamental Methods, ed. Gamborg and Phillips (Springer-Verlag, Berlin); McCabe et al. (1988) Biotechnology 6:923-926); and Lec1 transformation (WO 00/28058). Also see Weissinger et al. (1988) Ann. Rev. Genet. 22:421-477; Sanford et al. (1987) Particulate Science and Technology 5:27-37 (onion); Christou et al. (1988) Plant Physiol. 87:671-674 (soybean); McCabe et al. (1988) Bio/Technology 6:923-926 (soybean); Finer and McMullen (1991) In Vitro Cell Dev. Biol. 27P:175-182 (soybean); Singh et al. (1998) Theor. Appl. Genet. 96:319-324 (soybean); Datta et al. (1990) Biotechnology 8:736-740 (rice); Klein et al. (1988) Proc. Natl. Acad. Sci. USA 85:4305-4309 (maize); Klein et al. (1988) Biotechnology 6:559-563 (maize); U.S. Pat. Nos. 5,240,855; 5,322,783; and, 5,324,646; Klein et al. (1988) Plant Physiol. 91:440-444 (maize); Fromm et al. (1990) Biotechnology 8:833-839 (maize); Hooykaas-Van Slogteren et al. (1984) Nature (London) 311:763-764; U.S. Pat. No. 5,736,369 (cereals); Bytebier et al. (1987) Proc. Natl. Acad. Sci. USA 84:5345-5349 (Liliaceae); De Wet et al. (1985) in The Experimental Manipulation of Ovule Tissues, ed. Chapman et al. (Longman, New York), pp. 197-209 (pollen); Kaeppler et al. (1990) Plant Cell Reports 9:415-418 and Kaeppler et al. (1992) Theor. Appl. Genet. 84:560-566 (whisker-mediated transformation); D'Halluin et al. (1992) Plant Cell 4:1495-1505 (electroporation); Li et al. (1993) Plant Cell Reports 12:250-255 and Christou and Ford (1995) Annals of Botany 75:407-413 (rice); Osjoda et al. (1996) Nature Biotechnology 14:745-750 (maize via Agrobacterium tumefaciens); all of which are herein incorporated by reference. “Stable transformation” is intended to mean that the nucleotide construct introduced into a plant integrates into the genome of the plant and is capable of being inherited by the progeny thereof.

The cells that have been transformed may be grown into plants in accordance with conventional ways. See, for example, McCormick et al. (1986) Plant Cell Reports 5:81-84. In this manner, the present invention provides transformed seed (also referred to as “transgenic seed”) having a polynucleotide of the invention, for example, an expression cassette of the invention, stably incorporated into their genome.

The present invention may be used to alter the AGPase large subunit of any plant species, including, but not limited to, monocots and dicots. Examples of plant species of interest include, but are not limited to, corn (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), camelina (Camelina sativa), millet (e.g., pearl millet (Pennisetum glaucum), proso millet (Panicum miliaceum), foxtail millet (Setaria italica), finger millet (Eleusine coracana)), sunflower (Helianthus annuus), quinoa (Chenopodium quinoa), chicory (Cichorium intybus), pea (Pisum sativum), tomato (Solanum lycopersicum), lettuce (Lactuca sativa), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oil palm (Elaeis guineensis), poplar (Populus spp.), eucalyptus (Eucalyptus spp.), oats (Avena sativa), barley (Hordeum vulgare), vegetables, ornamentals, and conifers.

In one embodiment, a construct containing a promoter that is operable in a plant cell, operably linked to a coding sequence encoding a modified AGPase large subunit protein of the present invention is used to transform a plant cell or cells. The transformed plant cell or cells are regenerated to produce transformed plants. These plants transformed with a construct comprising a functional promoter driving expression of a modified AGPase large subunit protein-encoding polynucleotide of the invention demonstrated increased plant yield, i.e., increased above-ground biomass and/or and/or increased harvestable biomass and/or increased seed yield. In some embodiments, increased plant yield, i.e., increased above-ground biomass and/or and/or increased harvestable biomass and/or increased seed yield is observed particularly under conditions of abiotic stress including heat and/or drought stress.

In one embodiment, genomic modification results in the production of a coding sequence encoding a modified AGPase large subunit protein that replaces the native AGPase large subunit protein-encoding gene sequence in genome-modified cells. The genome-modified plant cell or cells are regenerated to produce transformed plants. These genome-modified plants comprising one or more modified AGPase large subunit coding genes encoding a modified AGPase large subunit protein of the invention demonstrate increased plant yield, i.e., increased above-ground biomass and/or and/or increased harvestable biomass and/or increased seed yield. In some embodiments, increased plant yield, i.e., increased above-ground biomass and/or and/or increased harvestable biomass and/or increased seed yield is observed particularly under conditions of abiotic stress including heat and/or drought stress. In some embodiments, the genomic modification is site-specific, e.g., through the use of site-specific nucleases such as CRISPR nucleases, TALENs, zinc-finger nucleases, meganucleases, or other site-specific nucleases, optionally combined with a repair donor template. In other embodiments, the genomic modification is not site-specific, e.g., through the use of chemical mutagens or radiation. Whether the mutation strategy is site-specific or not, molecular screening is performed following the mutation step to identify cells in which the desired mutation(s) have occurred.

Now that it has been demonstrated that expression of genes encoding modified AGPase large subunit proteins increases plant yield, other methods for expressing modified AGPase large subunit protein-encoding genes in a plant of interest can be used. The sequence of an AGPase large subunit protein-encoding gene present in a plant's genome can be altered by site-specific modifications, e.g., using meganucleases, zinc-finger nucleases, TALENs, CRISPR/Cas9, CRISPR/Cpf1, CRISPR/Cms1, CRISPR/C2c1, or other site-specific nucleases that produce double-stranded breaks at predetermined locations. These double-stranded breaks can then be repaired using a repair donor template that comprises the desired mutation(s). In some embodiments, the repair donor template is a single-stranded DNA (ssDNA) molecule, a double-stranded DNA (dsDNA) molecule, an RNA molecule, or a molecule comprising both DNA and RNA. This strategy will result in a modified AGPase large subunit protein-encoding sequence while maintaining the expression level and profile of the native AGPase large subunit protein-encoding gene.

Modulation of the expression of a modified AGPase large subunit protein-encoding gene may be achieved through the use of precise genome-editing technologies to modulate the expression of the sequence. In this manner, a nucleic acid sequence will be inserted proximal to a sequence encoding the modified AGPase large subunit protein through the use of methods available in the art. Such methods include, but are not limited to, meganucleases designed against the plant genomic sequence of interest (D'Halluin et al (2013) Plant Biotechnol J 11: 933-941); CRISPR-Cas9, CRISPR-Cpf1, CRISPR-Cms1, TALENs, and other technologies for precise editing of genomes (Feng et al. (2013) Cell Research 23:1229-1232, Podevin et al. (2013) Trends Biotechnology 31: 375-383, Wei et al. (2013) J Gen Genomics 40: 281-289, Zhang et al (2013) WO 2013/026740, Zetsche et al. (2015) Cell 163:759-771, U.S. Pat. No. 9,896,696); N. gregoryi Argonaute-mediated DNA insertion (Gao et al. (2016) Nat Biotechnol doi:10.1038/nbt.3547); Cre-lox site-specific recombination (Dale et al. (1995) Plant J 7:649-659; Lyznik, et al. (2007) Transgenic Plant J 1:1-9; FLP-FRT recombination (Li et al. (2009) Plant Physiol 151:1087-1095); Bxb1-mediated integration (Yau et al. (2011) Plant J 701:147-166); zinc-finger mediated integration (Wright et al. (2005) Plant J 44:693-705); Cai et al. (2009) Plant Mol Biol 69:699-709); and homologous recombination (Lieberman-Lazarovich and Levy (2011) Methods Mol Biol 701: 51-65; Puchta (2002) Plant Mol Biol 48:173-182). The insertion of said nucleic acid sequences will be used to achieve the desired result of overexpression, decreased expression, and/or altered expression profile of a modified AGPase large subunit-protein encoding gene.

Enhancers include any molecule capable of enhancing gene expression when inserted into the genome of a plant. Thus, an enhancer can be inserted in a region of the genome upstream or downstream of a modified AGPase large subunit protein-encoding sequence of interest to enhance expression. Enhancers may be cis-acting, and can be located anywhere within the genome relative to a gene for which expression will be enhanced. For example, an enhancer may be positioned within about 1 Mbp, within about 100 kbp, within about 50 kbp, about 30 kbp, about 20 kbp, about 10 kbp, about 5 kbp, about 3 kbp, or about 1 kbp of a coding sequence for which it enhances expression. An enhancer may also be located within about 1500 bp of a gene for which it enhances expression, or may be directly proximal to or located within an intron of a gene for which it enhances expression. Enhancers for use in modulating the expression of an endogenous gene encoding a modified AGPase large subunit protein or homolog according to the present invention include classical enhancer elements such as the CaMV 35S enhancer element, cytomegalovirus (CMV) early promoter enhancer element, and the SV40 enhancer element, and also intron-mediated enhancer elements that enhance gene expression such as the maize shrunken-1 enhancer element (Clancy and Hannah (2002) Plant Physiol. 130(2):918-29). Further examples of enhancers which may be introduced into a plant genome to modulate expression include a PetE enhancer (Chua et al. (2003) Plant Cell 15:11468-1479), or a rice α-amylase enhancer (Chen et al. (2002) J. Biol. Chem. 277:13641-13649), or any enhancer known in the art (Chudalayandi (2011) Methods Mol. Biol. 701:285-300). In some embodiments, the present invention comprises a subdomain, fragment, or duplicated enhancer element (Benfrey et al. (1990) EMBO J 9:1677-1684).

Alteration of AGPase large subunit gene expression may also be achieved through the modification of DNA in a way that does not alter the sequence of the DNA. Such changes could include modifying the chromatin content or structure of the AGPase large subunit gene of interest and/or of the DNA surrounding the AGPase large subunit gene. It is well known that such changes in chromatin content or structure can affect gene transcription (Hirschhorn et al. (1992) Genes and Dev 6:2288-2298; Narlikar et al. (2002) Cell 108: 475-487). Such changes could also include altering the methylation status of the AGPase large subunit gene of interest and/or of the DNA surrounding the AGPase large subunit gene of interest. It is well known that such changes in DNA methylation can alter transcription (Hsieh (1994) Mol Cell Biol 14: 5487-5494). Targeted epigenome editing has been shown to affect the transcription of a gene in a predictable manner (Hilton et al. (2015) 33: 510-517). It will be obvious to those skilled in the art that other similar alterations (collectively termed “epigenetic alterations”) to the DNA that regulates transcription of the AGPase large subunit gene of interest may be applied in order to achieve the desired result of an altered AGPase large subunit gene expression profile.

Alteration of AGPase large subunit gene expression may also be achieved through the use of transposable element technologies to alter gene expression. It is well understood that transposable elements can alter the expression of nearby DNA (McGinnis et al. (1983) Cell 34:75-84). Alteration of the expression of a gene encoding a modified AGPase large subunit protein may be achieved by inserting a transposable element upstream of the AGPase large subunit gene of interest, causing the expression of said gene to be altered.

Alteration of AGPase large subunit gene expression may also be achieved through expression of a transcription factor or transcription factors that regulate the expression of the AGPase large subunit gene of interest. It is well understood that alteration of transcription factor expression can in turn alter the expression of the target gene(s) of said transcription factor (Hiratsu et al. (2003) Plant J 34:733-739). Alteration of AGPase large subunit gene expression may be achieved by altering the expression of transcription factor(s) that are known to interact with an AGPase large subunit gene of interest (e.g., ZmbZIP; Chen et al. (2016) J Exp Bot 67:1327-1338).

Alteration of AGPase large subunit gene expression may also be achieved through the insertion of a promoter upstream of the open reading frame encoding an AGPase large subunit protein in the plant species of interest. This will occur through the insertion of a promoter of interest upstream of an AGPase large subunit protein-encoding open reading frame using a site-specific nuclease designed against the genomic sequence of interest. This strategy is well-understood and has been demonstrated previously to insert a transgene at a predefined location in the cotton genome (D'Halluin et al. (2013) Plant Biotechnol J 11: 933-941). It will be obvious to those skilled in the art that multiple technologies can be used to achieve a result of insertion of genetic elements at a predefined genomic locus by causing a double-strand break at said predefined genomic locus and providing an appropriate DNA template for insertion (e.g., meganucleases, CRISPR-Cas9, CRISPR-Cpf1, CRISPR-Cms1, TALENs, and other technologies for precise editing of genomes).

The following examples are offered by way of illustration and not by way of limitation. All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

### EXPERIMENTAL

### Example 1—Editing the Endogenous Maize Sh2 Gene

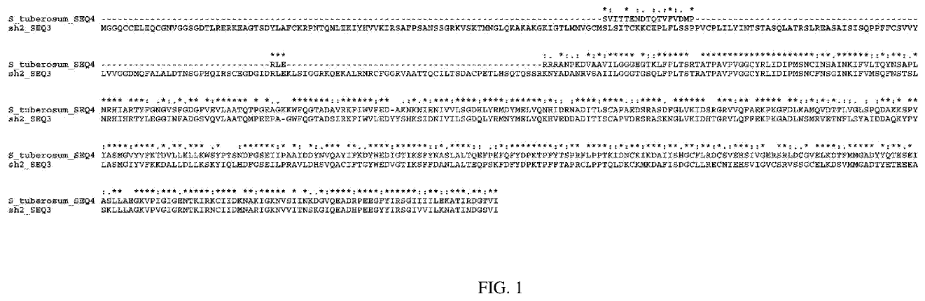

The maize sh2 gene (SEQ ID NO:1) comprises twenty exons; the processed cDNA (SEQ ID NO:2) encodes a 672 amino acid protein (SEQ ID NO:3) that is the large subunit of the AGPase holoenzyme found in the maize endosperm. Experiments with the sh2 gene and its encoded protein have uncovered multiple mutations that can result in improved thermostability and enzymatic activity when the mutant AGPase enzymes are expressed in a heterologous system. The ism2 mutant, for example (Boehlein et al. (2015) Arch Biochem Biophys 568:28-37), contains three mutations relative to the wild-type sh2 protein: Q252G, D317G, and A599R. The codons that encode the amino acids at residues 252, 317, and 599 are found in exons 7, 8, and 18, respectively. Separately, experiments with an AGPase large subunit protein from potato (SEQ ID NO:4) identified two beneficial mutations termed UpReg-1 and UpReg-2 (Greene et al. (1998) Proc Natl Acad Sci USA 95:10322-10327). An amino acid alignment of SEQ ID NOs:3 and 4 (FIG. 1) showed that the UpReg-1 and UpReg-2 mutations (E38K and G101N) corresponded to T249K and G312N mutations, respectively.

Genome editing is used to precisely modify the maize sh2 gene to encode mutant proteins. Table 2 summarizes the modified sh2 genes that are produced using genome editing.

TABLE 2
modifications made to the maize sh2 gene

| | Exons | | Encoded |
|---|---|---|---|
| Mutations | Modified | DNA sequence | protein |
| T249K + Q252G | 7 | SEQ ID NO: 5 | SEQ ID NO: 6 |
| G312N + D317G | 8 | SEQ ID NO: 7 | SEQ ID NO: 8 |
| T249K + Q252G + G312N + | 7, 8 | SEQ ID NO: 9 | SEQ ID NO: 10 |
| D317G | | | |
| T249K + Q252G + G312N + | 7, 8, 18 | SEQ ID NO: 11 | SEQ ID NO: 12 |
| D317G + A599R | | | |
| T249K + Q252G + A599R | 7, 18 | SEQ ID NO: 13 | SEQ ID NO: 14 |
| G312N + D317G + A599R | 8, 18 | SEQ ID NO: 15 | SEQ ID NO: 16 |

The edits summarized in Table 2 are produced using CRISPR endonucleases, coupled with appropriate guide RNAs, to produce site-specific double-stranded breaks (DSBs) near the intended sequence modifications. A single-stranded DNA (ssDNA) repair template is also provided. This ssDNA repair template has homology with the sequences up- and down-stream of the DSB site, and contains the mutations in the codons encoding amino acids number 249, 252, 312, 317, and/or 599, as appropriate. CRISPR endonucleases and guide RNAs are provided as ribonucleoproteins (RNPs) or as DNA constructs encoding the endonuclease and guide RNA(s); the repair donor template is provided as ssDNA. These components are delivered to maize callus cells biolistically by coating onto gold or tungsten beads. Following bombardment, the callus tissue is placed on tissue culture medium that is suitable for regeneration of plants; this tissue culture medium may contain a selective agent, as appropriate, if a selectable marker gene is included in the CRISPR endonuclease or guide RNA construct(s). Without being limited by theory, DSB production by the CRISPR endonuclease results in the maize cell DNA repair response; the repair donor template may be used for homology-directed repair (HDR), resulting in the incorporation of the desired mutations in the maize genomic DNA.

### Example 2—Molecular Characterization of Modified Maize Plants

Following delivery of the CRISPR endonuclease (or encoding polynucleotide), guide RNA (or encoding polynucleotide), and repair donor template DNA to maize callus tissue, cells from this callus tissue are harvested and DNA is extracted from these cells for analysis. Suitable molecular assays are performed to determine whether the desired genomic modifications are present in the maize cells. Such molecular assays may include, without limitation, PCR assays, next-generation sequencing (NGS) assays, restriction fragment length polymorphism (RFLP) assays, Taqman assays, and Sanger sequencing analyses. Maize cells whose genomic DNA contains the desired modifications are cultured and plants are regenerated from these cells for further molecular, biochemical, and phenotypic analysis.

### Example 3—Editing the Endogenous Rice AGPase Large Subunit Gene

The rice AGPase large subunit gene (SEQ ID NO:68) comprises fifteen exons; the processed cDNA (SEQ ID NO:67) encodes a 518 amino acid AGPase large subunit protein (SEQ ID NO:24). The amino acids in the rice AGPase large subunit that correspond to the maize sh2 T249, Q252, G132, D317, and A599 residues were identified based on amino acid alignments of SEQ ID NOs:2 and 24. Genome editing is used to precisely modify the rice AGPase large subunit gene to encode mutant proteins. Table 3 summarizes the modified rice AGPase large subunit genes that are produced using genome editing.

TABLE 3
modifications made to the rice AGPase large subunit gene

| | Exons | | Encoded |
|---|---|---|---|
| Mutations | Modified | DNA sequence | protein |
| T95K + Q98G | 2 | SEQ ID NO: 55 | SEQ ID NO: 49 |
| G158N + D163G | 3 | SEQ ID NO: 56 | SEQ ID NO: 50 |
| T95K + Q98G + | 2, 3 | SEQ ID NO: 57 | SEQ ID NO: 51 |
| G158N + D163G | | | |
| T95K + Q98G + G158N + | 2, 3, 13 | SEQ ID NO: 58 | SEQ ID NO: 52 |
| D163G + T445R | | | |
| T95K + Q98G + T445R | 2, 13 | SEQ ID NO: 59 | SEQ ID NO: 53 |
| G158N + D163G + T445R | 3, 13 | SEQ ID NO: 60 | SEQ ID NO: 54 |

The edits summarized in Table 3 are produced using CRISPR endonucleases, coupled with appropriate guide RNAs, to produce site-specific double-stranded breaks (DSBs) near the intended sequence modifications. Plasmids comprising:
1) an open reading frame encoding PbB14Cpf1 (Cpf1 nuclease derived from Prevotella bryantii B14; SEQ ID NO:69, encoding SEQ ID NO:70), driven by the CaMV 35S promoter (SEQ ID NO:71),
2) a guide RNA targeting the rice AGPase large subunit gene (SEQ ID NO:72), driven by the OsU6 promoter (SEQ ID NO:73),
3) a repair donor template (SEQ ID NO:74), and
4) a hygromycin resistance gene (SEQ ID NO:75, encoding SEQ ID NO:76), driven by the maize ubiquitin promoter (SEQ ID NO:77)

were used for biolistic transformation of rice callus tissue. The repair donor template (SEQ ID NO:74) comprised a number of mutations relative to the native rice sequence (SEQ ID NO:78). Of particular note, two primer pads were constructed in the introns of the AGPase gene with multiple mutations relative to the native rice sequence, allowing for ready detection of the desired mutations by PCR assays with primers designed to anneal with these primer pads.

The plasmids described above were delivered to rice callus biolistically using gold beads with previously described transformation protocols (U.S. Pat. No. 9,896,696). Following bombardment, the callus tissue was placed onto tissue culture medium containing hygromycin. Tissue samples were collected from these callus pieces and DNA was extracted from these tissue samples for molecular analysis.

### Example 4—Molecular Characterization of Modified Rice Plants

PCR reactions were performed using primers listed in Table 5 using DNA extracted from rice tissue samples. PCR was performed using the primer pairs of SEQ ID NOs:79 and 80, SEQ ID NOs:79 and 82, and SEQ ID NOs:80 and 83. While PCR performed using the primer pair of SEQ ID NOs:79 and 80 amplifies both wild-type and edited DNA, PCR performed using either the primer pair of SEQ ID NOs:79 and 82 or the primer pair of SEQ ID NOs:80 and 83 amplifies only edited rice DNA.

TABLE 5
Primers used to amplify edited rice DNA

| SEQ | | | |
|---|---|---|---|
| ID | | | |
| NO | Sequence | Direction | Anneals to |
| 79 | CCTCTTAGTTTGT | Forward | WT, upstream of |
| | AGGCGTATTCATG | | repair donor |
| | | | template |
| 80 | TTCTGATAGCATC | Reverse | WT, |
| | TGCTGTACCC | | downstream of |
| | | | repair donor |
| | | | template |
| 82 | TAGTGACATCTAG | Reverse | Edited DNA |
| | GTTGAGGTCG | | primer pad 1 |
| 83 | TGTGTACCTACGT | Forward | Edited DNA |
| | AGTGGTACC | | primer pad 2 |

Sanger sequencing was performed with the resulting PCR products to determine the sequence of the rice AGPase large subunit gene in these samples. Two callus pieces (callus pieces #7 and 14) were identified from this PCR and sequencing that comprised the intended mutations. Callus piece #7 showed the desired T95K, Q98G, G158N, and D163G mutations in both exon 2 and exon 3 (sequence identical to the repair donor template of SEQ ID NO:74), while callus piece #14 showed the desired exon 2 mutations (T95K and Q98G) but not the desired exon 3 mutations (G158N and D163G) (SEQ ID NO:81). Callus pieces whose genomic DNA contains the desired modifications are cultured and plants are regenerated from these cells for further molecular, biochemical, and phenotypic analysis.

### Example 5—Editing Additional AGPase Large Subunit Genes

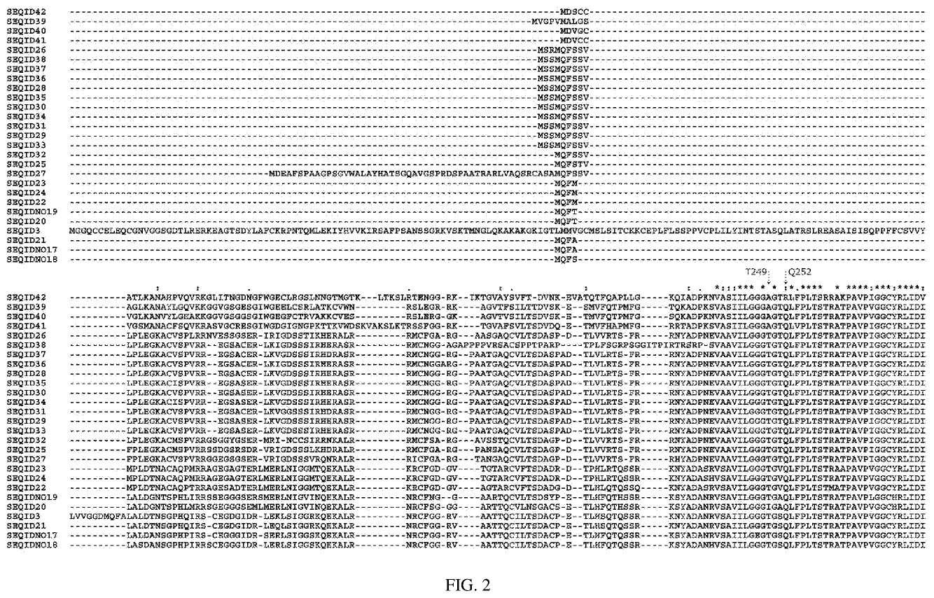

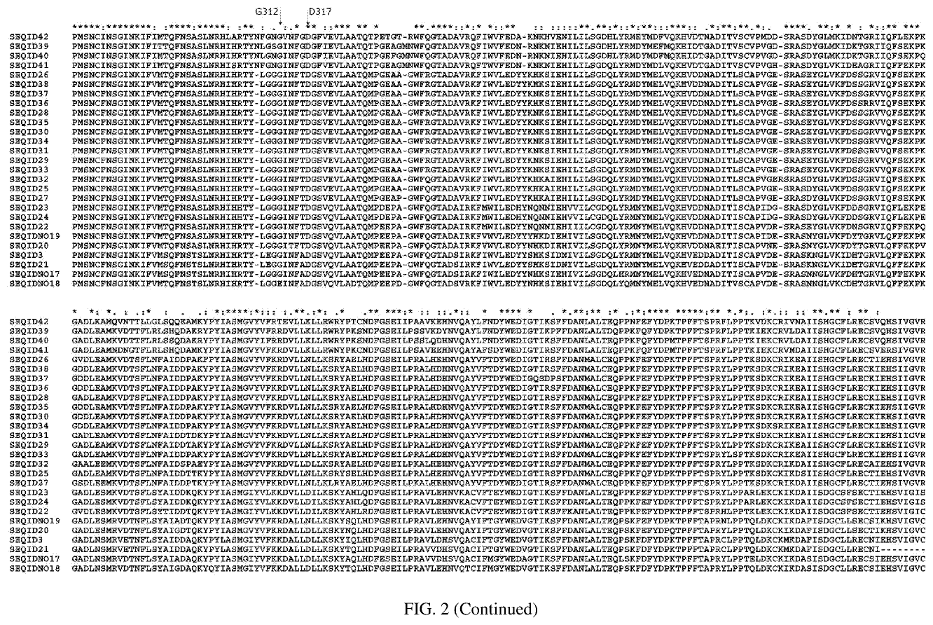

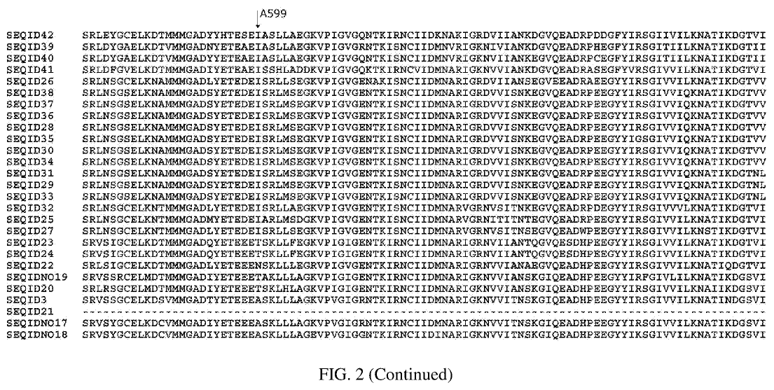

The modifications made to the maize sh2 gene (Table 2) and the rice AGPase large subunit gene (Table 3) are used to guide modifications to additional AGPase large subunit-encoding genes in maize and in other plant species. Exemplary AGPase large subunit proteins are listed as SEQ ID NOs:17-42. Amino acid alignments were performed among AGPase large subunit proteins of interest (SEQ ID NOs:17-42) and the maize sh2 protein sequence (SEQ ID NO:3), as shown in FIG. 2. Amino acid residues corresponding to the maize sh2 residue numbers 249, 252, 312, 317, and 599 (rice AGPase large subunit residues 95, 98, 158, 163, and 445) were identified from these alignments; these alignments are used to identify corresponding amino acid residues in additional AGPase large subunit sequences. Genomic DNA encoding the AGPase large subunit of interest is identified in the genome of the plant species of interest and the codons encoding these amino acids are identified. These codons are modified so that the amino acids corresponding to the maize sh2 residue numbers 249, 252, 312, 317, and 599 are modified to K, G, N, G, and R, respectively.

### Example 6—Cloning Modified AGPase Large Subunit Cassettes

The cDNA of the maize sh2 gene (SEQ ID NO:2) is modified to encode modified AGPase large subunit proteins with desired mutations. Table 6 summarizes the modifications that are made to the sh2 cDNA.

TABLE 6
Modified maize sh2 cDNA sequences

| Mutations | DNA sequence | Encoded protein |
|---|---|---|
| T249K + Q252G | SEQ ID NO: 43 | SEQ ID NO: 6 |
| G312N + D317G | SEQ ID NO: 44 | SEQ ID NO: 8 |
| T249K + Q252G + G312N + | SEQ ID NO: 45 | SEQ ID NO: 10 |
| D317G | | |
| T249K + Q252G + G312N + | SEQ ID NO: 46 | SEQ ID NO: 12 |
| D317G + A599R | | |
| T249K + Q252G + A599R | SEQ ID NO: 47 | SEQ ID NO: 14 |
| G312N + D317G + A599R | SEQ ID NO: 48 | SEQ ID NO: 16 |

The cDNA of the rice AGPase large subunit (SEQ ID NO:67) is modified to encode modified AGPase large subunit proteins with desired mutations. Table 7 summarizes the modifications that are made to the rice AGPase large subunit cDNA.

TABLE 7
Modified rice AGPase large subunit cDNA sequences

| Mutations | DNA sequence | Encoded protein |
|---|---|---|
| T95K + Q98G | SEQ ID NO: 61 | SEQ ID NO: 49 |
| G158N + D163G | SEQ ID NO: 62 | SEQ ID NO: 50 |
| T95K + Q98G + G158N + D163G | SEQ ID NO: 63 | SEQ ID NO: 51 |
| T95K + Q98G + G158N + | SEQ ID NO: 64 | SEQ ID NO: 52 |
| D163G + T445R | | |
| T95K + Q98G + T445R | SEQ ID NO: 65 | SEQ ID NO: 53 |
| G158N + D163G + T445R | SEQ ID NO: 66 | SEQ ID NO: 54 |

Modified AGPase large subunit genes are cloned in a plant transformation construct downstream from a promoter that is functional in plant cells and upstream from a terminator sequence that is functional in plant cells. Optionally, a selectable marker gene cassette designed to provide resistance to a selective agent (e.g., an herbicide or antibiotic) in transformed plant cells is included in the plant transformation construct.

Open reading frames encoding modified AGPase large subunit genes are constructed by first identifying an AGPase large subunit protein of interest (for example, an AGPase large subunit selected from the group consisting of SEQ ID NOs:17-42), then identifying the amino acid residues that correspond to T249, Q252, G312, D317, and A599 from the maize sh2 protein (SEQ ID NO:3) or that correspond to T95, Q98, G158, D163, and T445 from the rice AGPase large subunit protein (SEQ ID NO:24) based on amino acid alignments. Open reading frames encoding K, G, N, G, and R at these positions, respectively, are constructed. These open reading frames are cloned in a plant transformation construct downstream from a promoter that is functional in plant cells and upstream from a terminator sequence that is functional in plant cells. Optionally, a selectable marker gene cassette designed to provide resistance to a selective agent (e.g., an herbicide or antibiotic) in transformed plant cells is included in the plant transformation construct.

### Example 7—Expression of Modified AGPase Large Subunit Cassettes in Plants

The modified AGPase large subunit cassette-containing plant transformation constructs described in Example 6 are used to transform suitable tissue from a plant species of interest. Agrobacterium-mediated transformation, biolistic transformation, electroporation, PEG-mediated transformation, or other transformation methods may be used to introduce the plant transformation constructs into the plant cell(s). Following introduction of the plant transformation construct into plant cells, suitable tissue culture methods are used to regenerate transformed plants. In those cases where a selectable marker cassette is included in the plant transformation construct, a selective agent (e.g., a suitable herbicide or antibiotic) is included in the plant tissue culture medium at a concentration suitable to select for transformed plant cells.

Plants are regenerated from transformed plant cells. Tissue is sampled from the transformed plants and DNA is extracted from the regenerated plant tissue. This DNA is analyzed using standard molecular biology methods to determine whether the AGPase large subunit cassette is present in the plant genome. RNA and protein analyses are performed to determine whether the modified AGPase large subunit genes are expressed in the regenerated plants.

### Example 8—Use of Primer Pads to Detect Homology-Directed Repair (HDR) Events

The repair donor template of SEQ ID NO:74 comprises two primer pads. These “primer pads” are stretches of mutated DNA sequence that are closely linked to the intended DNA modification and may be used for PCR detection of the desired HDR-mediated genome editing events. The primer pads in SEQ ID NO:74 are annealed to by the primers of SEQ ID NOs:82 and 83 to allow for detection of the intended precise base changes in the O. sativa AGPase coding sequence.

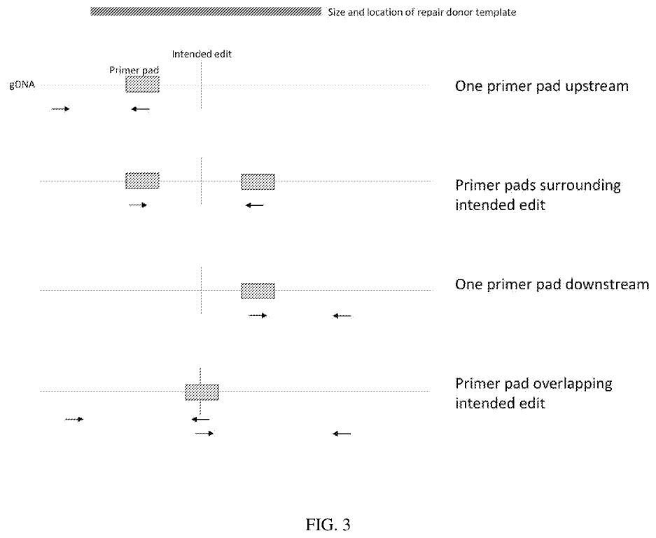

Primer pads are designed in non-coding regions of the genome (e.g., introns, intergenic regions, or other non-coding regions of DNA) or are designed in coding regions through the use of silent mutations to retain the encoded amino acid sequence. Typically, primer pads are positioned within fifty base pairs upstream or downstream of the intended HDR-mediated genome edits. In some embodiments, primer pads overlap with the intended HDR-mediated genome edits. At least one primer pad may be included in the repair donor template; in some embodiments, however, two or more primer pads may be included in the repair donor template. FIG. 3 shows a schematic design of primer pad locations and primer orientations that are used advantageously to detect precise genome editing modifications. In each case, at least one primer anneals to a primer pad; this primer does not anneal to unmodified wild-type DNA. In the schematics labeled “One primer pad upstream,” “One primer pad downstream,” and “Primer pad overlapping intended edit,” a second primer anneals outside of the repair donor template sequence and so anneals equally well with edited DNA and with wild-type DNA. Because the primer that anneals with the primer pad specifically anneals with edited DNA, however, the resulting PCR product is specific to edited events that comprise the desired edits. In the schematic labeled “Primer pads surrounding intended edit,” both primers anneal to primer pads that are located upstream and downstream, respectively, of the intended edit, resulting in specific detection of those events that comprise the primer pads both upstream and downstream of the intended edits. Because the primer pads are closely genetically linked, or in some cases even overlapping, the desired genome edit, PCR amplification of a sequence that comprises the primer pad (i.e. obtaining a PCR product when using one or more primers that anneals with a primer pad in the repair donor template) indicates an extremely high probability that the intended edit is also present in the sample.

We claim:

1. A modified AGPase large subunit protein comprising one or more non-native amino acid residues selected from:
a. Lysine at position 249 and glycine at position 252;
b. Asparagine at position 312 and glycine at position 317; or
c. Arginine at position 599,
where positions 249, 252, 312, 317, and 599 correspond to the amino acid numbering of the maize sh2 protein (SEQ ID NO:3), or a polynucleotide encoding said modified AGPase large subunit protein.

2. The modified AGPase large subunit protein of claim 1 wherein said AGPase large subunit protein shares at least 80% identity with SEQ ID NO:6, 8, 10, 12, 14, or 16, or is encoded by a polynucleotide that shares at least 70% identity with a sequence selected from the group consisting of SEQ ID NOs:5, 7, 9, 11, 13, 15, and 43-48.

3. A modified AGPase large subunit protein comprising one or more non-native amino acid residues selected from:
a. Lysine at position 95 and glycine at position 98;
b. Asparagine at position 158 and glycine at position 163; or
c. Arginine at position 445,
where positions 95, 98, 158, 163, and 445 correspond to the amino acid numbering of the rice AGPase large subunit protein (SEQ ID NO:24), or a polynucleotide encoding said modified AGPase large subunit protein.

4. The modified AGPase large subunit protein of claim 3 wherein said AGPase large subunit protein shares at least 80% identity with a sequence selected from the group consisting of SEQ ID NOs:49-54, or is encoded by a polynucleotide that shares at least 70% identity with a sequence selected from the group consisting of SEQ ID NOs:55-66.

5. The modified AGPase large subunit protein of claim 1 wherein said modified AGPase large subunit has at least 80% sequence identity to a sequence selected from the group consisting of SEQ ID NOs:17-42.

6. A plant transformation construct comprising a polynucleotide encoding the modified AGPase large subunit of claim 1, wherein said polynucleotide is operably linked to a promoter that is functional in a plant cell.

7. The plant transformation construct of claim 6 wherein said polynucleotide encoding the modified AGPase large subunit shares at least 70% sequence identity with a sequence selected from the group consisting of SEQ ID NOs:43-48 and 61-66, or encodes a protein that shares at least 80% sequence identity with a sequence selected from the group consisting of SEQ ID NOs:6, 8, 10, 12, 14, 16, and 49-54.

8. The plant transformation construct of claim 6 wherein said polynucleotide encoding the modified AGPase large subunit encodes a protein with at least 80% identity to a sequence selected from the group consisting of SEQ ID NOs:17-42.

9. A maize plant comprising a modified sh2 gene with at least 80% identity to a sequence selected from the group consisting of SEQ ID NOs:5, 7, 9, 11, 13, and 15.

10. A rice plant comprising a modified AGPase large subunit gene with at least 80% identity to a sequence selected from the group consisting of SEQ ID NOs:55-60.

11. A method of producing the modified AGPase large subunit protein of claim 1 in a plant cell comprising generating site-specific mutations at the codons encoding amino acids 249, 252, 312, 317 or 599 relative to SEQ ID NO:3, or at the codons encoding amino acids 95, 98, 158, 163, and 445 relative to SEQ ID NO:24 in the native AGPase large subunit gene in the genome of the plant in one or more plant cells.

12. The method of claim 11 wherein said site-specific mutations produce an AGPase large subunit gene with at least 80% identity to a sequence selected from the group consisting of SEQ ID NOs:5, 7, 9, 11, 13, 15, and 61-66.

13. The method of claim 11 further comprising regenerating a plant from said one or more plant cells.

14. A method of producing the modified AGPase large subunit protein of claim 1 in a plant cell comprising transforming one or more plant cells with the plant transformation construct of claim 6.

15. A plant produced by the method of claim 13.

16. A repair donor template molecule comprising a desired genome editing mutation and one one or more primer pads, wherein said primer pads comprise at least five primer pad mutations as compared to the targeted wild-type DNA sequence, and wherein said mutations comprise silent mutations in a coding region or are located in a non-coding region of DNA.

17. The repair donor template molecule of claim 16, wherein said repair donor template molecule comprises single-stranded DNA (ssDNA), double-stranded DNA (dsDNA), or circular DNA.

18. A method of detecting homology-directed repair (DR) mutation events comprising performing a polymerase chain reaction (PCR) using DNA extracted from one or more cells that has been exposed to a repair donor template comprising at least one primer pad and at least one primer designed to anneal to said primer pad, wherein said primer designed to anneal to said primer pad does not effectively anneal to the wild-type sequence.

19. A method of selecting cells that comprise a desired genomic modification comprising
a. detecting the presence of a desired mutation using the method of claim 18, and
b. allowing the cells from which the DNA was extracted to multiply,
and optionally further comprising allowing the cells from which the DNA was extracted to regenerate into a tissue or an organism.

20. The repair donor template of claim 16, wherein said repair donor template comprises nucleotides 235-252 or nucleotides 781-798 of SEQ ID NO:74.
